(19) United States
(12) Patent Application Publication
(10) Pub. No.: US 2018/0344869 A1
(43) Pub. Date: Dec. 6, 2018

(54) REDUCTION OF APPLICATION-RELATED SIDE REACTION OF A THERAPEUTIC ANTIBODY

(71) Applicant: Hoffmann-La Roche Inc., Little Falls, NJ (US)

(72) Inventors: Jens Fischer, Weilheim in Oberbayern (DE); Per-Ola Freskgard, Reinach BL (CH); Antonio Iglesias, Freiburg (DE); Jens Niewoehner, Muenchen (DE); Felix Weber, Grenzach-Wyhlen (DE)

(73) Assignee: Hoffmann-La Roche Inc., Little Falls, NJ (US)

(21) Appl. No.: 15/981,095

(22) Filed: May 16, 2018

(30) Foreign Application Priority Data
May 18, 2017 (EP) 17171626.9

(51) Int. Cl. A61K 47/68; A61P 25/00; A61K 35/00; C07K 16/28; A61K 39/395; A61K 47/65

(52) U.S. Cl. CPC A61K 47/6881; A61P 25/00; A61K 35/00; A61K 2039/507; A61K 39/395; A61K 47/65; A61K 47/6879; C07K 16/28

(57) ABSTRACT

The present invention relates to anti-brain target agents and therapeutic uses thereof.

### CROSS REFERENCE TO RELATED APPLICATIONS

This application claims benefit under 35 U.S.C. § 119 to European Patent Application No. 17171626.9, filed May 18, 2017, which is incorporated by reference herein in its entirety.

### SEQUENCE LISTING

A sequence listing is submitted concurrently with the specification as an ASCII formatted text file via EFS-Web, with a file name of “P34274-US_Sequence_Listing.txt,” a creation date of May 7, 2018, and a size of 126.621 bytes. The sequence listing filed via EFS-Web is part of the specification and is hereby incorporated by reference in its entirety herein.

### FIELD OF THE INVENTION

The present invention relates to therapeutic antibodies and uses thereof for treating disorders of the central nervous system.

### BACKGROUND

Disorders of the central nervous system (CNS) including, stroke, mental illness, neurodegenerative diseases, neurodevelopment disorders and brain tumors are the world's leading cause of disability. Although efforts to use conventional monoclonal antibodies (mAbs) are increasing, the blood-brain barrier (BBB) continues to hinder the development of effective therapies. As a consequence, technologies to overcome the BBB issue have received significant attention (1, 2). The development is nowadays focused on demonstrating substantial uptake and associated activity in brain at therapeutic dosing which reflects the delivery capacity. However, whether the technology being used conveys safety limitations to drug development is not always clear. Addressing this question upfront is crucial to identify aspects where design and protein and antibody engineering can facilitate safe delivery of mAbs to the brain.

BBB delivery utilizes natural receptors expressed on the brain endothelial cells (BECs) for transport purposes. In particular, the human transferrin receptor (TfR; also referred to as TfR1) has been extensively studied as a BBB delivery receptor due to the prominent expression at the BBB (3). Numerous groups have explored TfR as a receptor-mediated transcytosis (RMT) system for the delivery of molecules across the BBB (4-7). Recent efforts to engineer antibodies to allow productive and efficient crossing of the BBB have received increasing attention (8-11).

A Brain Shuttle (BS) technology using a bispecific antibody with two binding sites to a therapeutic target (i.e. which is bivalent for the therapeutic target) and one binding site to the TfR (i.e. which is monovalent for the human transferrin receptor 1) was developed to allow delivery of monoclonal antibodies (mAbs) with fully functional, i.e. therapeutic target binding as well as effector function competent, IgG structure. This is accomplished by fusing one BS module to the C-terminal end of one heavy chain of the mAb. By linking the BS module to an anti-amyloid-beta mAb (Aβ mAb) it has recently been demonstrated substantial improvement in brain exposure, target engagement and efficacy (9). This enhanced brain delivery was hypothesized to be a direct consequence of the natural monovalent engagement of the BS construct with the TfR.

In WO 2014/033074 blood brain barrier shuttles that bind receptors on the blood brain barrier (R/BBB) and methods of using the same are disclosed.

Increased brain penetration and potency of a therapeutic antibody using a monovalent molecular shuttle are disclosed by Niewoehner et al. (Neuron 81 (2014) 49-60; 9).

### SUMMARY

The inventors of the present invention identified a method for reducing application-related side effects and reactions of a bispecific therapeutic monoclonal antibody. This is achieved by sterically abrogating binding to Fcγ receptors (FcγRs). One example is a bispecific therapeutic antibody specifically binding to a therapeutic target related to a disorder of the central nervous system and the human transferrin receptor (TfR).

A recent study has revealed a liability previously overlooked using conventional mAbs against TfR (TfR1). Acute clinical signs were observed in mice directly after dosing and this was linked to the effector function status of the mAb (12). This was also observed when using bispecific mAbs where only one Fab arm binds to TfR (TfR1), provided the mAb contained a native fully active effector function. Taken together, the effector function of a mAb seems to be directly linked to the observed acute clinical signs, and so an obvious evading strategy would be to use an effector-dead variant. However, for certain mAbs a native effector function is crucial for the mode-of-action and optimal therapeutic profile.

It has now been found in in vitro and in vivo assessments of different formats of the Brain Shuttle-mAb (BS-mAb) system in a novel FcγR-humanized mouse model with respect to potential first infusion reaction (FIR) liability of native IgG effector function that the Fc-region effector function of TfR(TfR1)-targeting BS-mAbs is camouflaged when the mAb binds to TfR (TfR1) (and at the same time not binding to the therapeutic target) but is back to active when the mAb binds its CNS target (and at the same time not binding to the TfR (TfR1)) depending on the format of the BS-mAb.

Without being bound by this theory it is assumed that the observed format dependence of the FIR is due to steric factors influencing the binding/accessibility of the Fc-region to the FcγR located on immune cells. It is hypothesized that when TfR (TfR1) is bound by the BS module the two natural Fab arms at the opposite end of the BS-mAb prevent the required proximity of the Fc-region of the BS-mAb to the FcγR on effector cells. Once the BS-mAb is released from the TfR (TfR1), e.g. into the CNS parenchyma, and the resident target is bound by the native, therapeutic IgG Fabs, the free BS module at the heavy chain C-terminus does no longer influence with or prevent the interaction of the Fc-region with FcγR on recruited effector cells.

Thus, the teaching conveyed herein provides the basis for the selection and the use of fully effector-functional mAbs that can be transported safely across the BBB. Furthermore, it lends key considerations for future TfR (TfR1) targeting therapies focused on enhancing mAb uptake in the brain. The data as reported herein provides new teachings on the interaction between mAbs bound to their antigen on a first cell and the geometry in binding to an FcγR on a second cell. Thereby new mAb designs with reduced first injection reactions (FIR) can be provided and/or selected.

The present invention relates in one aspect to the use of a bispecific antibody that specifically binds to a first and a second (cell surface) target and that has (native) effector function in a specific format, in which the antibody has two binding sites (VH/VL pairs) that specifically bind to the first (cell surface) target, one binding site (VH/VL pair) that specifically binds to the second (cell surface) target and an effector function competent, e.g. native, Fc-region for the reduction of undesired administration(infusion)-related side effects (as vasodilation, bronchoconstriction, laryngeal edema, drop of cardiac pressure, and in particular of hypothermia associated with Fc-region effector function) in the treatment of a disease/disorder.

The present invention relates in one aspect to a therapeutic composition for use in a method for treatment of a disease comprising a bispecific antibody that specifically binds to a first and a second (cell surface) target and that has (native) effector function in a specific format, in which the antibody has two binding sites (VH/VL pairs) that specifically bind to the first (cell surface) target, one binding site (VH/VL pair) that specifically binds to the second (cell surface) target and an effector function competent, e.g. native, Fc-region, wherein the therapeutic composition has reduced undesired administration(infusion)-related side effects (as vasodilation, bronchoconstriction, laryngeal edema, drop of cardiac pressure, and in particular of hypothermia) associated with the Fc-region effector function.

The present invention relates in one aspect to a pharmaceutical composition comprising a therapeutic bispecific antibody for use in preventing and/or treating a disease that has undesired administration(infusion)-related side effects (as vasodilation, bronchoconstriction, laryngeal edema, drop of cardiac pressure, and in particular of hypothermia) associated with Fc-region effector function by administering a bispecific antibody that specifically binds to a first and a second (cell surface) target and that has (native) effector function in a specific format, in which the antibody has two binding sites (VH/VL pairs) that specifically bind to the first (cell surface) target, one binding site (VH/VL pair) that specifically binds to the second (cell surface) target and an effector function competent Fc-region.

The present invention relates in one aspect to a bispecific antibody for use in the treatment of a disease in a patient,
wherein the bispecific antibody comprises
   i) an (effector function competent) Fc-region,
   ii) two binding sites specifically binding to a first (cell surface) target, and
   iii) one binding site specifically binding to a second (cell surface) target,
wherein the treatment has reduced side effect after administration,
wherein the side effect is one or more selected from the group consisting of vasodilation, bronchoconstriction, laryngeal edema, drop of cardiac pressure, and hypothermia.

In other words, the present invention relates in one aspect to a bispecific antibody for use in the treatment of a disease in a patient and for reducing the side effect after administration,
wherein the bispecific antibody comprises
   i) an (effector function competent) Fc-region,
   ii) two binding sites specifically binding to a first (cell surface) target, and
   iii) one binding site specifically binding to a second (cell surface) target,
wherein the side effect is one or more selected from the group consisting of vasodilation, bronchoconstriction, laryngeal edema, drop of cardiac pressure, and hypothermia.

In one embodiment the two binding sites specifically binding to the first target and the binding site specifically binding to the second target are arranged in opposite directions, i.e. one is conjugated to the N-terminus of the Fc-region and the other is conjugated to the C-terminus of the Fc-region.

In one embodiment the first (cell surface) target and the second (cell surface) target are different.

In one embodiment the binding sites specifically binding to the first (cell surface) target and the binding site specifically binding to the second (cell surface) target are located at opposite ends (i.e. those specifically binding to the first target are both/each at an N-terminal end of a (full length) antibody heavy chain and that to the second target is at the C-terminal end of one of the (full length) antibody heavy chains of the bispecific antibody.

In one embodiment the binding sites specifically binding to the first (cell surface) target and the binding site specifically binding to the second (cell surface) target are located at opposite ends of the bispecific antibody, i.e. one of the binding sites specifically binding to the first target is conjugated to the first N-terminus of the Fc-region and the other is conjugated to the second N-terminus of the Fc-region and the binding site that specifically binds to the second target is conjugated to one of the C-termini of the Fc-region.

In one embodiment the administration-related side effects are infusion-related side effects. In one embodiment the infusion-related side effects are vasodilation, bronchoconstriction, laryngeal edema, drop of cardiac pressure, and hypothermia. In one preferred embodiment the infusion-related side effect is hypothermia.

In one embodiment the binding site specifically binding to the second (cell surface) target is linked to one of the binding sites specifically binding to the first (cell surface) target by a peptidic linker. In one embodiment the peptidic linker has the amino acid sequence of SEQ ID NO: 37 or 38.

In one embodiment the binding site specifically binding to a second (cell surface) target is within the Fc-region, wherein at least one structural loop region of any of a CH2 domain, a CH3 domain, or a CH4 domain comprises at least one modification enabling the binding of said at least one modified loop region to the second (cell surface) target wherein the unmodified immunoglobulin constant domain does not bind to said target.

In one embodiment the binding sites are pairs of an antibody heavy chain variable domain and an antibody light chain variable domain.

In one embodiment the bispecific antibody comprises
i) a pair of a first antibody light chain and a first antibody heavy chain,
ii) a pair of a second antibody light chain and a second antibody heavy chain, and
iii) an additional antibody fragment selected from the group consisting of scFv, Fab, scFab, dAb fragment, DutaFab and CrossFab,
wherein the pair of antibody chains of i) and ii) comprise the binding sites specifically binding to the first (cell surface) target and the additional antibody fragment of iii) comprises the binding site specifically binding to the second (cell surface) target.

In one embodiment the additional antibody fragment of iii) is conjugated either directly or via a peptidic linker either to the first antibody heavy chain or to the second antibody heavy chain. In one embodiment the additional antibody fragment of iii) is conjugated either directly or via a peptidic linker to the C-terminus of the antibody heavy chain of i) or ii). In one embodiment the peptidic linker has the amino acid sequence of SEQ ID NO: 37 or 38. In one embodiment the first antibody light chain and the second antibody light chain have the same amino acid sequence and the first antibody heavy chain and the second antibody heavy chain differ by mutations required for heterodimerization. In one embodiment the mutations required for heterodimerization are the knobs-into-hole mutations. In one embodiment the antibody heavy chain not conjugated to the additional antibody fragment of iii) does not comprise i) the C-terminal lysine residue or ii) the C-terminal glycine-lysine dipeptide.

In one embodiment the first target is a brain target and the second target is the human transferrin receptor. In one embodiment the first target is a brain target and the second target is the human transferrin receptor 1.

In one embodiment the brain target is selected from the group consisting of beta-secretase 1 (BACE1), human amyloid beta (Abeta), epidermal growth factor receptor (EGFR), human epidermal growth factor receptor 2 (HER2), human Tau protein, phosphorylated human Tau protein, apolipoprotein E4 (ApoE4), human alpha-synuclein, human CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6. In one preferred embodiment the brain target is selected from the group consisting of human CD20, human Tau protein, phosphorylated human Tau protein, human alpha-synuclein and human amyloid beta protein. In one preferred embodiment the brain target is human amyloid beta protein. In one embodiment the brain target is selected from SEQ ID NO: 01 to 05.

In one preferred embodiment the bispecific antibody in all aspects as reported herein comprises
i) a pair of a first antibody light chain and a first antibody heavy chain comprising a first light chain variable domain and a first heavy chain variable domain, which form a first binding site specifically binding to a brain target selected from the group consisting of human CD20, human Tau protein, phosphorylated human Tau protein, human alpha-synuclein and human amyloid beta protein,
ii) a pair of a second antibody light chain and a second antibody heavy chain comprising a second light chain variable domain and a second heavy chain variable domain, which form a second binding site specifically binding to the same brain target as the first binding site,
iii) an additional antibody fragment selected from the group consisting of scFv, Fab, scFab, dAb fragment, DutaFab and CrossFab, comprising a third light chain variable domain and a third heavy chain variable domain, which form a third binding site specifically binding to the human transferrin receptor (transferrin receptor 1), and
iv) a (human) effector function competent Fc-region (of the human IgG1 subclass),
wherein the additional antibody fragment of iii) is conjugated either directly or via a peptidic linker to the C-terminus of the antibody heavy chain of i) or ii).

In one embodiment the additional antibody fragment is a Fab fragment, which specifically bind to a second antigen, and which is fused via a peptidic linker to the C-terminus of one of the heavy chains of i) or ii), wherein the constant domains CL and CH1 of the second light chain and the second heavy chain are replaced by each other, comprising a third light chain variable domain and a third heavy chain variable domain, which form a third binding site specifically binding to the human transferrin receptor (transferrin receptor 1).

In one embodiment the binding site specifically binding to the human transferrin receptor (transferrin receptor 1) comprises (a) a HVR-H1 comprising the amino acid sequence of SEQ ID NO: 06 or 07; (b) a HVR-H2 comprising the amino acid sequence of SEQ ID NO: 08 or 09 or 10; (c) a HVR-H3 comprising the amino acid sequence of SEQ ID NO: 11, 12 or 13; (d) a HVR-L1 comprising the amino acid sequence of SEQ ID NO: 14 or 15; (e) a HVR-L2 comprising the amino acid sequence of SEQ ID NO: 16; and (f) a HVR-L3 comprising the amino acid sequence of SEQ ID NO: 17 or 18.

In one embodiment the binding site specifically binding to the human transferrin receptor (transferrin receptor 1) comprises (a) a HVR-H1 comprising the amino acid sequence of SEQ ID NO: 06; (b) a HVR-H2 comprising the amino acid sequence of SEQ ID NO: 08; (c) a HVR-H3 comprising the amino acid sequence of SEQ ID NO: 12; (d) a HVR-L1 comprising the amino acid sequence of SEQ ID NO: 14; (e) a HVR-L2 comprising the amino acid sequence of SEQ ID NO: 16; and (f) a HVR-L3 comprising the amino acid sequence of SEQ ID NO: 18.

In one embodiment the antibody comprises one pair of a heavy chain variable domain of SEQ ID NO: 19 and a light chain variable domain of SEQ ID NO: 20 forming a binding site for the transferrin receptor (transferrin receptor 1) and at least one (i.e. one or two) pair of a heavy chain variable domain of SEQ ID NO: 23 and a light chain variable domain of SEQ ID NO: 24 (each) forming a binding site for human amyloid beta protein (Abeta).

In one embodiment the antibody comprises one pair of a heavy chain variable domain of SEQ ID NO: 19 and a light chain variable domain of SEQ ID NO: 20 forming the binding site for the human transferrin receptor (transferrin receptor 1) and two pairs of a heavy chain variable domain of SEQ ID NO: 21 and a light chain variable domain of SEQ ID NO: 22 each forming a binding site for human CD20. In one embodiment, the heavy chain variable region comprises a replacement of the amino acid residue at Kabat position 11 with any amino acid but leucine. In one embodiment, the substitution comprises a replacement of the amino acid residue at Kabat position 11 with a nonpolar amino acid. In one preferred embodiment, the substitution comprises a replacement of the amino acid residue at Kabat position 11 in the heavy chain variable domain of SEQ ID NO: 21 with an amino acid residue selected from the group consisting of valine, leucine, isoleucine, serine, and phenylalanine.

In one embodiment the antibody comprises one pair of a heavy chain variable domain of SEQ ID NO: 19 and a light chain variable domain of SEQ ID NO: 20 forming the binding site for the human transferrin receptor (transferrin receptor 1) and two pairs of a heavy chain variable domain of SEQ ID NO: 25 and a light chain variable domain of SEQ ID NO: 26 each forming a binding site for human alpha-synuclein.

In one embodiment the antibody comprises one pair of a heavy chain variable domain of SEQ ID NO: 19 and a light chain variable domain of SEQ ID NO: 20 forming the binding site for the human transferrin receptor (transferrin receptor 1) and two pairs of a humanized heavy chain variable domain derived from SEQ ID NO: 27 and a humanized light chain variable domain derived from SEQ ID NO: 28 each forming a binding site for human alpha-synuclein.

In one embodiment the antibody comprises one pair of a heavy chain variable domain of SEQ ID NO: 19 and a light chain variable domain of SEQ ID NO: 20 forming the binding site for the human transferrin receptor and two pairs of a humanized heavy chain variable domain derived from SEQ ID NO: 29 and a humanized light chain variable domain derived from SEQ ID NO: 30 each forming a binding site for human alpha-synuclein.

In one embodiment the antibody comprises one pair of a heavy chain variable domain of SEQ ID NO: 19 and a light chain variable domain of SEQ ID NO: 20 forming the binding site for the human transferrin receptor (transferrin receptor 1) and two pairs of a humanized heavy chain variable domain derived from SEQ ID NO: 31 and a humanized light chain variable domain derived from SEQ ID NO: 32 each forming a binding site for human alpha-synuclein.

In one embodiment the antibody comprises one pair of a heavy chain variable domain of SEQ ID NO: 19 and a light chain variable domain of SEQ ID NO: 20 forming the binding site for the human transferrin receptor (transferrin receptor 1) and two pairs of a humanized heavy chain variable domain derived from SEQ ID NO: 33 and a humanized light chain variable domain derived from SEQ ID NO: 34 each forming a binding site for human alpha-synuclein.

In one embodiment the antibody comprising one pair of a heavy chain variable domain of SEQ ID NO: 19 and a light chain variable domain of SEQ ID NO: 20 forming the binding site for the human transferrin receptor (transferrin receptor 1) and two pairs of a humanized heavy chain variable domain derived from SEQ ID NO: 35 and a humanized light chain variable domain derived from SEQ ID NO: 36 each forming a binding site for human alpha-synuclein.

In one embodiment the disease is a neurological disorder. In one embodiment the disease is selected from the group of neurological disorders consisting of neuropathy, amyloidosis, cancer, an ocular disease or disorder, viral or microbial infection, inflammation, ischemia, neurodegenerative disease, seizure, behavioral disorders, lysosomal storage disease, Lewy body disease, post poliomyelitis syndrome, Shy-Draeger syndrome, olivopontocerebellar atrophy, Parkinson's disease, multiple system atrophy, striatonigral degeneration, tauopathies, Alzheimer disease, supranuclear palsy, prion disease, bovine spongiform encephalopathy, scrapie, Creutzfeldt-Jakob syndrome, kuru, Gerstmann-Straussler-Scheinker disease, chronic wasting disease, and fatal familial insomnia, bulbar palsy, motor neuron disease, nervous system heterodegenerative disorder, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, Unverricht-Lundborg syndrome, dementia, Pick's disease, spinocerebellar ataxia, cancer of the CNS and/or brain, including brain metastases resulting from cancer elsewhere in the body. In one embodiment the disease is selected from the group of neurological disorders consisting of Alzheimer's disease, Parkinson's disease, cancer of the CNS and/or brain, including brain metastases resulting from cancer elsewhere in the body, and tauopathies. In one embodiment the disease is selected from the group of neurological disorders consisting of Alzheimer's disease, Parkinson's disease and tauopathies.

In one embodiment the antibody comprises an effector function competent Fc-region. In one embodiment the effector function competent Fc-region is an Fc-region that specifically binds to/can be specifically bound by human Fcgamma receptor. In one embodiment the effector function competent Fc-region can elicit ADCC.

In one embodiment ADCC elicited (upon injection/while binding to the second (cell surface) target) by the bispecific antibody is lower than that elicited by a bivalent bispecific antibody that has only one, i.e. exactly one, binding site that specifically bind to the first (cell surface) target and (exactly) one binding site that specifically binds to the second (cell surface) target, i.e. one of the binding sites specifically binding to the first (cell surface) target is deleted. In one embodiment the ADCC is 10-fold or more lower.

In one embodiment the administration is an intravenous, subcutaneous, or intramuscular administration.

In one embodiment the administration-related side effect is hypothermia. In one embodiment the hypothermia is reduced to a drop of body-temperature of less than 0.5° C. at a therapeutic dose of the bispecific antibody. In one embodiment the drop of the body temperature is within 60 minutes after administration.

In one embodiment the first antibody heavy chain (of i)) and the second antibody heavy chain (of ii)) form a heterodimer. In one embodiment the first antibody heavy chain and the second antibody heavy chain comprise mutations supporting the formation of a heterodimer.

In one embodiment
a) the antibody heavy chains are full length antibody heavy chains of the human subclass IgG1,
b) the antibody heavy chains are full length antibody heavy chains of the human subclass IgG4,
c) one of the antibody heavy chains is a full length antibody heavy chain of the human subclass IgG1 with the mutations T366W and optionally S354C or Y349C and the other antibody heavy chain is a full length antibody heavy chain of the human subclass IgG1 with the mutations T366S, L368A, Y407V and optionally Y349C or S354C,
d) both antibody heavy chains are full length antibody heavy chains of the human subclass IgG1 with the mutations I253A, H310A and H435A and the mutations T366W and optionally S354C or Y349C in one of the antibody heavy chains and the mutations T366S, L368A, Y407V and optionally Y349C or S354C in the respective other antibody heavy chain,
e) both antibody heavy chains are full length antibody heavy chains of the human subclass IgG1 with the mutations M252Y, S254T and T256E and the mutations T366W and optionally S354C or Y349C in one of the antibody heavy chains and the mutations T366S, L368A, Y407V and optionally Y349C or S354C in the respective other antibody heavy chain, or
f) both antibody heavy chains are antibody heavy chains of the human subclass IgG1 with the mutations T307H and N434H and the mutations T366W and optionally S354C or Y349C in one of the antibody heavy chains and the mutations T366S, L368A, Y407V and optionally Y349C or S354C in the respective other antibody heavy chain.

In one embodiment
a) the antibody heavy chains are antibody heavy chains of the human subclass IgG1,
b) the antibody heavy chains are antibody heavy chains of the human subclass IgG4,
c) one of the antibody heavy chains is an antibody heavy chain of the human subclass IgG1 with the mutations T366W and optionally S354C or Y349C and the other antibody heavy chain is an antibody heavy chain of the human subclass IgG1 with the mutations T366S, L368A, Y407V and optionally Y349C or S354C,
d) both antibody heavy chains are antibody heavy chains of the human subclass IgG1 with the mutations I253A, H310A and H435A and the mutations T366W and optionally S354C or Y349C in one of the antibody heavy chains and the mutations T366S, L368A, Y407V and optionally Y349C or S354C in the respective other antibody heavy chain,
e) both antibody heavy chains are antibody heavy chains of the human subclass IgG1 with the mutations M252Y, S254T and T256E and the mutations T366W and optionally S354C or Y349C in one of the antibody heavy chains and the mutations T366S, L368A, Y407V and optionally Y349C or S354C in the respective other antibody heavy chain, or
f) both antibody heavy chains are antibody heavy chains of the human subclass IgG1 with the mutations T307H and N434H and the mutations T366W and optionally S354C or Y349C in one of the antibody heavy chains and the mutations T366S, L368A, Y407V and optionally Y349C or S354C in the respective other antibody heavy chain,
wherein the C-terminal lysine or glycine-lysine dipeptide is present or absent.

The present invention relates to the use of bispecific antibodies that specifically bind to a brain target and to the human transferrin receptor 1 and that have native effector function in a specific format, in which the antibody has two binding sites (VH/VL pairs) that specifically bind to the brain target, one binding site (VH/VL pair) that specifically binds to the human transferrin receptor 1 and an effector function competent, e.g. native, Fc-region, for the reduction of undesired administration(infusion)-related side effects as vasodilation, bronchoconstriction, laryngeal edema, drop of cardiac pressure, and in particular of hypothermia associated with Fc-region effector function, in the treatment of a neurological disorder. This antibody is a fully effector-functional antibody that can be transported across the blood-brain barrier.

It is believed, without being bound by this theory, that binding of the therapeutic antibody at the same time to human Fcgamma receptor on an effector cell as well as to the human transferrin receptor (transferrin receptor 1) on any TfR(TfR1)-expressing cell of the body may at least be partly responsible for the observed anaphylactoid reactions after infusion thereof. By providing the therapeutic antibody in a specific format, which prevents undesired Fc-receptor interactions off target, the occurrence of administration (infusion)-related side-effects, especially of hypothermia, can be reduced or even prevented.

Furthermore, a clinical benefit of reducing the anaphylactoid reactions is expected to allow a better tolerance and/or higher administration(infusion)-rates or doses of the therapeutic antibody.

As discussed herein above, new mAb designs with reduced first injection reactions (FIR) are provided. This in turn allows for the application of higher dosages, more frequent dosing and/or higher infusion rates of the bispecific therapeutic antibody or the therapeutic composition comprising the bispecific therapeutic antibody as compared to administration schemes of other therapeutic bispecific antibody formats. Similarly, in accordance with present invention, in patients who experience undesired administration(infusion)-related side effects (as vasodilation, bronchoconstriction, laryngeal edema, drop of cardiac pressure, and in particular of hypothermia), the dosage, dosing frequency and/or infusion rate do not have to be reduced as in existing therapies.

In conventional antibody therapies, when patients receiving an antibody therapy experience administration (infusion)-related side-effects (also referred herein as infusion-related reaction), the infusion rate needs to be lowered or in severe cases the therapy needs to be interrupted or discontinued entirely. This can be avoided with the present invention. For patients experiencing mild or moderate infusion related reactions (e.g. grades 1 and 2 according to the Common Terminology Criteria for Adverse Events (CTCAE) v5.0 of the United States National Cancer Institute (NCI)), the infusion rate may be decreased. Patients experiencing severe infusion related side effects (e.g. grades 3 and 4 according to the Common Terminology Criteria for Adverse Events (CTCAE) v5.0 of the United States National Cancer Institute (NCI)), the therapy must be stopped immediately and finally discontinued. The present invention provides a therapy that can be safely administered to avoid such side reactions at all or at least greatly reduce such side reactions.

Hence, in some aspects the invention is used to treat patients that would otherwise experience administration(infusion)-related side effects (such as vasodilation, bronchoconstriction, laryngeal edema, drop of cardiac pressure, and in particular of hypothermia), particularly administration(infusion)-related side effects of grades 1 to 4 (according to the Common Terminology Criteria for Adverse Events (CTCAE) v5.0 of the United States National Cancer Institute (NCI)), more in particular grades 2 to 4, and more in particular grades 3 and 4.

For example, typical infusion rates for patients without infusion-related side effects may for some antibodies be between 12 ml/h and 400 ml/h (e.g. an infusion may start at the first (and optionally second) administration with a rate of 12 ml/h and is doubled every 30 min until a rate of 200 ml/h is reached; the third and subsequent infusions may, e.g. be started at a rate of 25 mg/l which is doubled every 30 min until a maximum infusion rate of 400 ml/h is reached). In conventional antibody therapies, for patients experiencing mild or moderate infusion related reactions, the infusion may in this example be interrupted, later resumed at 12 ml/h and slowly increased under the supervision of a physician. As discussed, this can be avoided with the present invention.

It has been found that both therapeutic target binding Fab arms are required to maximize the inhibitory effect on FcγR recruitment in order to minimize administration(infusion)-related drop of the body-temperature and cytokine release.

Thus, one aspect as reported herein is an anti-brain target therapeutic agent, which is an anti-brain target/human transferrin receptor (transferrin receptor 1) (bispecific) antibody, wherein the anti-brain target/human transferrin receptor (1) antibody has two binding sites (VH/VL pairs) that specifically bind to the brain target, one binding site (VH/VL pair) that specifically binds to the human transferrin receptor (transferrin receptor 1) and an effector function competent (native) Fc-region, for use in anti-brain target treatment in an individual with reduced undesired infusion-related side effect, such as vasodilation, bronchoconstriction, laryngeal edema, drop of cardiac pressure, and in particular of hypothermia, after intravenous application.

Another aspect as reported herein is a method for treating a neurological disorder with reduced infusion-related side effects, such as vasodilation, bronchoconstriction, laryngeal edema, drop of cardiac pressure, and in particular hypothermia in an individual comprising the administration of an effective amount of an anti-brain target/human transferrin receptor (transferrin receptor 1) (bispecific) antibody, wherein the anti-brain target/human transferrin receptor (transferrin receptor 1) antibody has two binding sites (VH/VL pairs) that specifically bind to the brain target, one binding site (VH/VL pair) that specifically binds to the human transferrin receptor (transferrin receptor 1) and an effector function competent (native) Fc-region, wherein the treatment results in a reduced infusion-related side effect, such as vasodilation, bronchoconstriction, laryngeal edema, drop of cardiac pressure, and in particular of hypothermia.

In one embodiment the infusion-related side-effect is hypothermia, i.e. a drop in body temperature.

The antibody employed in the aspect described above can be any antibody as described herein.

In one embodiment the hypothermia is reduced to a drop of body-temperature of less than 2° C. In one embodiment the hypothermia is reduced to a drop of body-temperature of less than 1° C. In one preferred embodiment the hypothermia is reduced to a drop of body temperature of less than 0.5° C.

In one embodiment the hypothermia is within 30 minutes after administration. In one embodiment the hypothermia is within 60 minutes after administration. In one embodiment the hypothermia is within 120 minutes after administration.

In one embodiment the hypothermia is reduced to a drop of body-temperature of less than 1° C., in one preferred embodiment less than 0.5° C., within 60 minutes, in one preferred embodiment within 120 minutes, after administration.

In one embodiment the effector function competent Fc-region is an Fc-region that specifically binds to/can be specifically bound by a human Fcgamma receptor.

In one embodiment the effector function competent Fc-region can elicit ADCC.

In one embodiment the effector function competent Fc-region is an Fc-region that specifically binds to/can be specifically bound by human Fcgamma receptor and can elicit ADCC.

In one embodiment the anti-brain target/human transferrin receptor 1 antibody is a trivalent, bispecific antibody, comprising
i) a first light chain and a first heavy chain of a full length antibody which specifically binds to a first antigen,
ii) a second heavy chain of a full length antibody which when paired with the first light chain, specifically binds to the first antigen, and
iii) a Fab fragment, which specifically bind to a second antigen, and which is fused via a peptidic linker to the C-terminus of one of the heavy chains of i) or ii), wherein the constant domains CL and CH1 of the second light chain and the second heavy chain are replaced by each other,
wherein the C-terminal lysine or glycine-lysine dipeptide is present or absent.

In one preferred embodiment the bispecific antibody in all aspects as reported herein comprises
i) a pair of a first antibody light chain and a first antibody heavy chain comprising a first light chain variable domain and a first heavy chain variable domain, which form a first binding site specifically binding to a brain target selected from the group consisting of human CD20, human Tau protein, phosphorylated human Tau protein, human alpha-synuclein and human amyloid beta protein,
ii) a pair of a second antibody light chain and a second antibody heavy chain comprising a second light chain variable domain and a first heavy chain variable domain, which form a second binding site specifically binding to the same brain target as the first binding site,
iii) an additional antibody fragment selected from the group consisting of scFv, Fab, scFab, dAb fragment, and CrossFab, comprising a third light chain variable domain and a third heavy chain variable domain, which form a third binding site specifically binding to the human transferrin receptor (transferrin receptor 1), and
iv) a (human) effector function competent Fc-region,
wherein the additional antibody fragment of iii) is conjugated either directly or via a peptidic linker to the C-terminus of the antibody heavy chain of i) or ii),
wherein the C-terminal lysine or glycine-lysine dipeptide is present or absent.

In one embodiment the binding site specifically binding to the human transferrin receptor (transferrin receptor 1) comprises (a) a HVR-H1 comprising the amino acid sequence of SEQ ID NO: 06 or 07; (b) a HVR-H2 comprising the amino acid sequence of SEQ ID NO: 08 or 09 or 10; (c) a HVR-H3 comprising the amino acid sequence of SEQ ID NO: 11, 12 or 13; (d) a HVR-L1 comprising the amino acid sequence of SEQ ID NO: 14 or 15; (e) a HVR-L2 comprising the amino acid sequence of SEQ ID NO: 16; and (f) a HVR-L3 comprising the amino acid sequence of SEQ ID NO: 17 or 18.

In one embodiment the binding site specifically binding to the human transferrin receptor (transferrin receptor 1) comprises (a) a HVR-H1 comprising the amino acid sequence of SEQ ID NO: 06; (b) a HVR-H2 comprising the amino acid sequence of SEQ ID NO: 08; (c) a HVR-H3 comprising the amino acid sequence of SEQ ID NO: 12; (d) a HVR-L1 comprising the amino acid sequence of SEQ ID NO: 14; (e) a HVR-L2 comprising the amino acid sequence of SEQ ID NO: 16; and (f) a HVR-L3 comprising the amino acid sequence of SEQ ID NO: 18.

In one embodiment the antibody comprises one pair of a heavy chain variable domain of SEQ ID NO: 19 and a light chain variable domain of SEQ ID NO: 20 forming a binding site for the transferrin receptor (transferrin receptor 1) and at least one (i.e. one or two) pair of a heavy chain variable domain of SEQ ID NO: 23 and a light chain variable domain of SEQ ID NO: 24 forming a binding site for human amyloid beta protein (Abeta).

In one embodiment the antibody comprises one pair of a heavy chain variable domain of SEQ ID NO: 19 and a light chain variable domain of SEQ ID NO: 20 forming the binding site for the human transferrin receptor (transferrin receptor 1) and two pairs of a heavy chain variable domain of SEQ ID NO: 21 and a light chain variable domain of SEQ ID NO: 22 each forming a binding site for human CD20. In one embodiment, the heavy chain variable region comprises a replacement of the amino acid residue at Kabat position 11 with any amino acid but leucine. In one embodiment, the substitution comprises a replacement of the amino acid residue at Kabat position 11 with a nonpolar amino acid. In one preferred embodiment, the substitution comprises a replacement of the amino acid residue at Kabat position 11 in the heavy chain variable domain of SEQ ID NO: 21 with an amino acid residue selected from the group consisting of valine, leucine, isoleucine, serine, and phenylalanine.

In one embodiment the antibody comprises one pair of a heavy chain variable domain of SEQ ID NO: 19 and a light chain variable domain of SEQ ID NO: 20 forming the binding site for the human transferrin receptor (transferrin receptor 1) and two pairs of a heavy chain variable domain of SEQ ID NO: 25 and a light chain variable domain of SEQ ID NO: 26 each forming a binding site for human alpha-synuclein.

In one embodiment the antibody comprises one pair of a heavy chain variable domain of SEQ ID NO: 19 and a light chain variable domain of SEQ ID NO: 20 forming the binding site for the human transferrin receptor (transferrin receptor 1) and two pairs of a humanized heavy chain variable domain derived from SEQ ID NO: 27 and a humanized light chain variable domain derived from SEQ ID NO: 28 each forming a binding site for human alpha-synuclein.

In one embodiment the antibody comprises one pair of a heavy chain variable domain of SEQ ID NO: 19 and a light chain variable domain of SEQ ID NO: 20 forming the binding site for the human transferrin receptor (transferrin receptor 1) and two pairs of a humanized heavy chain variable domain derived from SEQ ID NO: 29 and a humanized light chain variable domain derived from SEQ ID NO: 30 each forming a binding site for human alpha-synuclein.

In one embodiment the antibody comprises one pair of a heavy chain variable domain of SEQ ID NO: 19 and a light chain variable domain of SEQ ID NO: 20 forming the binding site for the human transferrin receptor (transferrin receptor 1) and two pairs of a humanized heavy chain variable domain derived from SEQ ID NO: 31 and a humanized light chain variable domain derived from SEQ ID NO: 32 each forming a binding site for human alpha-synuclein.

In one embodiment the antibody comprises one pair of a heavy chain variable domain of SEQ ID NO: 19 and a light chain variable domain of SEQ ID NO: 20 forming the binding site for the human transferrin receptor (transferrin receptor 1) and two pairs of a humanized heavy chain variable domain derived from SEQ ID NO: 33 and a humanized light chain variable domain derived from SEQ ID NO: 34 each forming a binding site for human alpha-synuclein.

In one embodiment the antibody comprising one pair of a heavy chain variable domain of SEQ ID NO: 19 and a light chain variable domain of SEQ ID NO: 20 forming the binding site for the human transferrin receptor (transferrin receptor 1) and two pairs of a humanized heavy chain variable domain derived from SEQ ID NO: 35 and a humanized light chain variable domain derived from SEQ ID NO: 36 each forming a binding site for human alpha-synuclein.

In one embodiment the disease is a neurological disorder. In one embodiment the disease is selected from the group of neurological disorders consisting of neuropathy, amyloidosis, cancer, an ocular disease or disorder, viral or microbial infection, inflammation, ischemia, neurodegenerative disease, seizure, behavioral disorders, lysosomal storage disease, Lewy body disease, post poliomyelitis syndrome, Shy-Draeger syndrome, olivopontocerebellar atrophy, Parkinson's disease, multiple system atrophy, striatonigral degeneration, tauopathies, Alzheimer disease, supranuclear palsy, prion disease, bovine spongiform encephalopathy, scrapie, Creutzfeldt-Jakob syndrome, kuru, Gerstmann-Straussler-Scheinker disease, chronic wasting disease, and fatal familial insomnia, bulbar palsy, motor neuron disease, nervous system heterodegenerative disorder, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, Unverricht-Lundborg syndrome, dementia, Pick's disease, spinocerebellar ataxia, cancer of the CNS and/or brain, including brain metastases resulting from cancer elsewhere in the body. In one embodiment the disease is selected from the group of neurological disorders consisting of Alzheimer's disease, Parkinson's disease, cancer of the CNS and/or brain, including brain metastases resulting from cancer elsewhere in the body, and tauopathies. In one embodiment the disease is selected from the group of neurological disorders consisting of Alzheimer's disease, Parkinson's disease and tauopathies.

In one embodiment the antibody comprises an effector function competent Fc-region. In one embodiment the effector function competent Fc-region is an Fc-region that specifically binds to/can be specifically bound by human Fcgamma receptor. In one embodiment the effector function competent Fc-region can elicit ADCC.

In one embodiment ADCC elicited (upon injection/while binding to the second (cell surface) target) by the bispecific antibody is lower than that elicited by a bivalent bispecific antibody that has only one, i.e. exactly one, binding site that specifically bind to the first (cell surface) target and (exactly) one binding site that specifically binds to the second (cell surface) target. In one embodiment the ADCC is 10-fold or more lower.

In one embodiment the administration is an intravenous, subcutaneous, or intramuscular administration.

In one embodiment the administration-related side effect is hypothermia. In one embodiment the hypothermia is reduced to a drop of body-temperature of less than 0.5° C. at a therapeutic dose of the bispecific antibody. In one embodiment the drop of the body temperature is within 60 minutes after administration.

In one embodiment the first antibody heavy chain (of i)) and the second antibody heavy chain (of ii)) form a heterodimer. In one embodiment the first antibody heavy chain and the second antibody heavy chain comprise mutations supporting the formation of a heterodimer.

In one embodiment the full length antibody is
a) a full length antibody of the human subclass IgG1,
b) a full length antibody of the human subclass IgG4,
c) a full length antibody of the human subclass IgG1 with the mutations T366W and optionally S354C in one heavy chain and the mutations T366S, L368A, Y407V and optionally Y349C in the respective other heavy chain,
d) a full length antibody of the human subclass IgG1 with the mutations I253A, H310A and H435A in both heavy chains and the mutations T366W and optionally S354C in one heavy chain and the mutations T366S, L368A, Y407V and optionally Y349C in the respective other heavy chain,
e) a full length antibody of the human subclass IgG1 with the mutations M252Y, S254T and T256E in both heavy chains and the mutations T366W and optionally S354C in one heavy chain and the mutations T366S, L368A, Y407V and optionally Y349C in the respective other heavy chain, or
f) both antibody heavy chains are antibody heavy chains of the human subclass IgG1 with the mutations T307H and N434H and the mutations T366W and optionally S354C in one of the antibody heavy chains and the mutations T366S, L368A, Y407V and optionally Y349C in the respective other antibody heavy chain.

In one embodiment
a) the antibody heavy chains are antibody heavy chains of the human subclass IgG1,
b) the antibody heavy chains are antibody heavy chains of the human subclass IgG4,
c) one of the antibody heavy chains is an antibody heavy chain of the human subclass IgG1 with the mutations T366W and optionally S354C and the other antibody heavy chain is an antibody heavy chain of the human subclass IgG1 with the mutations T366S, L368A, Y407V and optionally Y349C,
d) both antibody heavy chains are antibody heavy chains of the human subclass IgG1 with the mutations I253A, H310A and H435A and the mutations T366W and optionally S354C in one of the antibody heavy chains and the mutations T366S, L368A, Y407V and optionally Y349C in the respective other antibody heavy chain,
e) both antibody heavy chains are antibody heavy chains of the human subclass IgG1 with the mutations M252Y, S254T and T256E and the mutations T366W and optionally S354C in one of the antibody heavy chains and the mutations T366S, L368A, Y407V and optionally Y349C in the respective other antibody heavy chain, or
f) both antibody heavy chains are antibody heavy chains of the human subclass IgG1 with the mutations T307H and N434H and the mutations T366W and optionally S354C in one of the antibody heavy chains and the mutations T366S, L368A, Y407V and optionally Y349C in the respective other antibody heavy chain,
wherein the C-terminal lysine or glycine-lysine dipeptide is present or absent.

In one embodiment the human effector function competent Fc-region comprises two polypeptides selected from the group consisting of SEQ ID NO: 57 to 60 and 63 to 66.

In one embodiment the human effector function competent Fc-region comprises a first Fc-region polypeptide of SEQ ID NO: 61 and a second Fc-region polypeptide of SEQ ID NO: 62.

As used herein the term “aspect” denotes an independent subject of the current invention whereas the term “embodiment” denotes a further defined, dependent sub-item of an independent subject.

### BRIEF DESCRIPTION OF THE FIGURES

FIGS. 1A-1E show that in vitro FcγR binding and Fc-region function is conserved in the BS-mAb31 construct when free in solution. FIG. 1A illustrates a Brain Shuttle construct binding to an FcγR on the cell surface in free solution, the structure includes FcγR, Fc-region, Fabs and the BS module. FIG. 1B depicts surface plasmon resonance (SPR) sensogram showing immobilization of the different FcγRs (first signal) and binding of the anti-Abeta antibody mAb31 thereto (second signal). FIG. 1C depicts surface plasmon resonance (SPR) sensogram showing immobilization of the different FcγRs (first signal) and binding of the BS-anti-Abeta antibody mAb31 (BS-mAb31) thereto (second signal). FIG. 1D depicts cell binding of mAb31 (open symbols) and BS-mAb31 (filled symbols) to either the huFcγRI (triangle) or huFcγRIIIa (circle) demonstrating that both constructs have comparable affinity to these two FcγRs and stronger to the high affinity huFcγRI. FIG. 1E depicts cell binding of mAb31 (open symbols) and BS-mAb31 (filled symbols) to either the huFcγRIIa (square) or huFcγRIIb (diamond) showing that both constructs have comparable affinity to these two low affinity huFcγRs.

FIGS. 2A-2K show that in vitro FcγR binding and Fc-region function is conserved in the BS-mAb31 construct when engaged in AP target binding. FIG. 2A illustrates a Brain Shuttle construct binding to an FcγR when anti-Aβ Fab arms bound to Amyloid-3. The structure includes FcγR, Fc-region, Fabs and the BS module. FIGS. 2B and 2C depict in vitro ADCC activity of mAb31 and BS-mAb31 measuring IL-8 release (FIG. 2B) or IP-10 release (FIG. 2C) using AP 1-42 coated surface and U937 monocyte effector cells. Both constructs have similar ADCC activity. FIGS. 2D-2K show Cellular phagocytosis of human Aβ plaques. Human AD brain sections were pre-incubated with either mAb31 (FIGS. 2D-2G) or BS-mAb31 (FIGS. 2H-2K), followed by cell culturing in presence of primary human macrophages as effector cells. Equimolar concentration used was 0 μg/ml (FIGS. 2D and 2H), 1 μg/ml (FIGS. 2E and 2I), 5 μg/ml (FIGS. 2F and 2J) and 5 μg/ml (FIGS. 2G and 2K) without primary human macrophages. Plaques were labeled afterwards with an anti-Aβ antibody. Data shows similar concentration-dependent phagocytosis activity and in vitro plaque clearance for both constructs.

FIGS. 3A-3F illustrate in vivo target engagement and amyloid-β reduction for BS-mAb31 construct. FIG. 3A shows pharmacokinetics performed in C57BL6 male mice and that the plasma exposure was lower for the BS-mAb31 compared to mAb31. The lower exposure of the BS molecule is attributed to binding to TfR1 in the periphery. FIG. 3B shows that chronic dosing profiles were then simulated using pharmacokinetic parameters determined from the single dose PK data at the appropriate doses used. FIGS. 3C-3D show that plaque binding was assessed after the final 4 months' dose for mAb31 (FIG. 3C) and BS-mAb31 (FIG. 3D). APPLondon mice treated with mAb31 or BS-mAb31 for 4 months. Plaque load of untreated animals sacrificed at an age of 17.5 months is shown for comparison as baseline level of amyloidosis at study begins. FIGS. 3E-3F show that strong and significant reduction in plaque number is evident after treatment with BS-mAb31, both on cortex (FIG. 3E) and hippocampus (FIG. 3F), compared to the progressive plaque formation seen in the vehicle and mAb31 group.

FIGS. 4A-4E illustrate that the orientation of TfR1 bound BS-mAb31 display the Fc-region in a non-optimal position for productive FcγR interaction on an adjacent cell. FIGS. 4A and 4B are schematic illustrations of a Brain Shuttle construct (FIG. 4A) or a standard anti-TfR1 IgG mAb (FIG. 4B) binding to the TfR1 on the cell surface. TfR1, Tf, BS module, Fc-region and cargo Fabs (therapeutic binding sites). FIG. 4C shows cytotoxicity curves of different constructs. Anti-TfR1 IgG1 antibody elicited ADCC of BaF3 target cells whereas the BS constructs have attenuated activity. Standard anti-TfR1 mAb (circle), Standard anti-TfR1 mAb with one Fab (black square with error bars), BS-2Fab (triangle with error bars), dBS-IgG (reverse triangle), control IgG (diamond), standard anti-TfR1 mAb PGLALA (grey square). FIG. 4D shows total cytotoxicity values for each construct at a concentration with the maximum effect of the standard anti-TfR1 mAb; only the standard anti-TfR1 mAb with one Fab shows a small effect while all other constructs have no detectable ADCC activity. All constructs contain a fully functional human IgG1 Fc-region. FIG. 4E shows schematic overview of the constructs investigated in the ADCC assay. Values plotted are means±SD (n=3). ****p≤0.0001 (t-test, compared to standard anti-TfR1 mAb-dosed animals).

FIGS. 5A-5E show that a standard anti-TfR1 mAb with effector function induces first infusion reaction and cytokine induction. FIG. 5A illustrates an overview on the design of the FcγR-humanized mice model. Gene-targeted FcγR locus exchange. FIG. 5B illustrates that the temperature changes in mice were monitored with a wireless system using a capsule injected under the skin; allowed the animals to move freely during the study. FIG. 5C shows that the FIR can be elicited in FcγR-humanized mice and is characterized by a drop in body temperature. The standard anti-TfR1 mAb induced dose-dependent transient temperature drop at 5 mg/kg (circle) and 20 mg/kg (square), vehicle control (triangle). FIG. 5D shows that the FIR response requires a fully active effector function as the standard anti-TfR1 mAb PGLALA (filled circle) induce no temperature drop at 20 mg/kg. Standard anti-TfR1 mAb (filled triangle) and vehicle (open triangle) was included as controls. FIG. 5E shows that a panel of cytokines in the blood was monitored 2 hours post injection. There was a strong increase for certain cytokines in the standard anti-TfR1 mAb (shaded bars) group which was almost diminished in the group with no effector function (standard anti-TfR1 mAb PGLALA, open bars).

FIGS. 6A-6E show that an anti-TfR1 Brain Shuttle construct with effector function attenuates first infusion reaction and cytokine induction. FIG. 6A shows three different Brain Shuttle constructs engineered and produced for testing. The difference between the constructs is the deletion of the cargo Fabs to investigate how they influence FcγR engagement in vivo when the constructs binds to TfR1. FIG. 6B shows the results when the three constructs were tested at 5 mg/kg in the same study. The mBS-2Fab (BS-mAb; triangle) induced no FIR whereas the construct lacking both cargo Fabs had the strongest effect (square). The one cargo Fab construct (circle) was in between the other two constructs. FIG. 6C shows % cytokine response for a panel of cytokines in the blood 2 hours post injection of the constructs. Only mBS-noFab (open bars) induced a strong induction of certain cytokines whereas the mBS-2Fab (BS-mAb; shaded bars) had no substantial effect. FIG. 6D compares two doses of the BS-sFab, 5 mg/kg (open triangle) and 20 mg/kg (filled circle) and a vehicle group (filled triangle). There was a small and a very transient temperature drop at 20 mg/kg for the BS-sFab. FIG. 6E shows FIR monitored by temperature drop for the standard anti-TfR1 mAb (circle) compared to the BS-noFab construct. Interestingly, the BS-noFab (square) was much more potent inducing FIRs. A vehicle group (triangle) was included.

FIGS. 7A-7B illustrate that a distinct cytokine pattern is induced by a standard anti-TfR1 mAb with effector function which is diminished for the Brain Shuttle construct. In FIG. 7A, the reference coloring (scale) is shown. The heatmap (FIGS. 7B-1 and 7B-2) shows an overview at a 5 mg/kg dose for various constructs. It shows the temperature-cytokine relationship for two cytokines and the various constructs. The heatmap was generated to highlight key cytokines. In particular, two cytokines responded very differently.

FIGS. 8A-8B show that a standard anti-TfR1 mAb with effector function induce ROS activation which is mitigated using the Brain Shuttle construct. FIG. 8A shows detection of ROS induction using whole body imaging. FIG. 8B depicts quantification of ROS production showing that only the anti-TfR1 mAb induce a strong reaction, which is in agreement with the FIR data.

FIGS. 9A-9F illustrate molecular modeling of the putative FcγR/TfR1 binding modes. FIGS. 9A and 9D represent standard IgG (optionally with C-terminal anti-TfR1 CrossFab fusion); FIGS. 9B and 9E represent BS-noFab (Fc-anti-TfR1 CrossFab C-terminal fusion); and FIGS. 9C and 9F represent BS-mAb (mBS-2Fab; targeted IgG-anti-mTfR1 CrossFab C-terminal fusion). FIGS. 9A-9C show the side view with the effector cell and the FcγR thereon on top, and the respective target (TfR and plaque, respectively) on the bottom. FIGS. 9D-9F show the top view onto the basolateral side of the effector cell membrane and approximate how multiple of the complexes shown in FIGS. 9A-9C might cluster laterally in the plane of the interaction partners. FIGS. 9A and 9D show that the interaction of the standard IgG with the FcγR on the effector cell is possible while the standard IgG is bound to its therapeutic target. FIGS. 9A and 9D also show that the presence of an additional BS-module (anti-TfR1 CrossFab) at the C-terminus of the standard IgG does not interfere with the FcγR binding. FIGS. 9C and 9F show that the interaction of the BS-mAb with the FcγR on the effector cell is not possible while the BS-mAb is bound to the TfR.

### DETAILED DESCRIPTION OF EMBODIMENTS OF THE INVENTION

The human transferrin receptor (TfR) (transferrin receptor 1, TfR1) has shown promise for transport of antibodies (mAbs) across the blood-brain barrier (BBB). However, safety liabilities have been reported associated with peripheral TfR(TfR1)-binding and Fc-region effector function. The Brain Shuttle-mAb (BS-mAb) technology was used to investigate the role of Fc-region effector function in vitro and in a novel FcγR-humanized mouse model. Strong first injection reactions (FIR) were observed for a conventional bivalent monospecific mAb against TfR (TfR1) with a native IgG1 Fc-region. Using Fc-region effector-dead constructs completely eliminated all FIR. Remarkably, no FIR was observed for the 2+1 BS-mAb construct with a native IgG1 Fc-region. The invention as reported herein is based at least in part on the finding that TfR (TfR1) binding through the C-terminal BS-module attenuates Fc-region-FcγR interactions, primarily due to steric hindrance. Nevertheless, BS-mAbs maintain effector function activity when it binds its target. Taken together, mAbs with full effector function can be transported in a stealth mode in the periphery and become activated in the brain only when engaged with its target.

### Definitions

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, Md. (1991) is used for the light chain constant domain CL of kappa and lambda isotype, and the Kabat EU index numbering system (see pages 661-723) is used for the constant heavy chain domains (CH1, Hinge, CH2 and CH3, which is herein further clarified by referring to “numbering according to Kabat EU index” in this case).

The knobs into holes dimerization modules and their use in antibody engineering are described in Carter P.; Ridgway J. B. B.; Presta L. G.: Immunotechnology, Volume 2, Number 1, February 1996, pp. 73-73(1).

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991).

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the thereafter following numerical value. In one embodiment the term about denotes a range of +/−10% of the thereafter following numerical value. In one embodiment the term about denotes a range of +/−5% of the thereafter following numerical value.

The term “determine” as used herein encompasses also the terms measure and analyze.

The term “domain crossover” as used herein denotes that in a pair of an antibody heavy chain VH-CH1 fragment and its corresponding cognate antibody light chain, i.e. in an antibody binding arm (i.e. in the Fab fragment), the domain sequence deviates from the natural sequence in that at least one heavy chain domain is substituted by its corresponding light chain domain and vice versa. There are three general types of domain crossovers, (i) the crossover of the CH1 and the CL domains, which leads to domain crossover light chain with a VL-CH1 domain sequence and a domain crossover heavy chain fragment with a VH-CL domain sequence (or a full length antibody heavy chain with a VH-CL-hinge-CH2-CH3 domain sequence), (ii) the domain crossover of the VH and the VL domains, which leads to domain crossover light chain with a VH-CL domain sequence and a domain crossover heavy chain fragment with a VL-CH1 domain sequence, and (iii) the domain crossover of the complete light chain (VL-CL) and the complete VH-CH1 heavy chain fragment (“Fab crossover”), which leads to a domain crossover light chain with a VH-CH1 domain sequence and a domain crossover heavy chain fragment with a VL-CL domain sequence (all aforementioned domain sequences are indicated in N-terminal to C-terminal direction).

As used herein the term “replaced by each other” with respect to corresponding heavy and light chain domains refers to the aforementioned domain crossovers. As such, when CH1 and CL domains are “replaced by each other” it is referred to the domain crossover mentioned under item (i) and the resulting heavy and light chain domain sequence. Accordingly, when VH and VL are “replaced by each other” it is referred to the domain crossover mentioned under item (ii); and when the CH1 and CL domains are “replaced by each other” and the VH1 and VL domains are “replaced by each other” it is referred to the domain crossover mentioned under item (iii). Bispecific antibodies including domain crossovers are reported, e.g. in WO 2009/080251, WO 2009/080252, WO 2009/080253, WO 2009/080254 and Schaefer, W. et al, Proc. Natl. Acad. Sci USA 108 (2011) 11187-11192.

The multispecific antibody comprises Fab fragments including a domain crossover of the CH1 and the CL domains as mentioned under item (i) above, or a domain crossover of the VH and the VL domains as mentioned under item (ii) above. The Fab fragments specifically binding to the same antigen(s) are constructed to be of the same domain sequence. Hence, in case more than one Fab fragment with a domain crossover is contained in the multispecific antibody, said Fab fragment(s) specifically bind to the same antigen.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “antibody-dependent cellular cytotoxicity (ADCC)” is a function mediated by Fc receptor binding and refers to lysis of target cells by an antibody as reported herein in the presence of effector cells. ADCC is measured in one embodiment by the treatment of a preparation of CD19 expressing erythroid cells (e.g. K562 cells expressing recombinant human CD19) with an antibody as reported herein in the presence of effector cells such as freshly isolated PBMC (peripheral blood mononuclear cells) or purified effector cells from buffy coats, like monocytes or NK (natural killer) cells. Target cells are labeled with ⁵¹Cr and subsequently incubated with the antibody. The labeled cells are incubated with effector cells and the supernatant is analyzed for released ⁵¹Cr. Controls include the incubation of the target endothelial cells with effector cells but without the antibody. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA) In one preferred embodiment binding to FcγR on NK cells is measured.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; dAb fragments; linear antibodies; single-chain antibody molecules (e.g. scFv); and multispecific antibodies formed from antibody fragments.

The term “complement-dependent cytotoxicity (CDC)” refers to lysis of cells induced by the antibody as reported herein in the presence of complement. CDC is measured in one embodiment by the treatment of CD19 expressing human endothelial cells with an antibody as reported herein in the presence of complement. The cells are in one embodiment labeled with calcein. CDC is found in one embodiment if the antibody induces lysis of 20% or more of the target cells at a concentration of 30 μg/ml. Binding to the complement factor C1q can be measured in an ELISA. In such an assay in principle an ELISA plate is coated with concentration ranges of the antibody, to which purified human C1q or human serum is added. C1q binding is detected by an antibody directed against C1q followed by a peroxidase-labeled conjugate. Detection of binding (maximal binding Bmax) is measured as optical density at 405 nm (OD405) for peroxidase substrate ABTS® (2,2′-azino-di-[3-ethylbenzthiazoline-6-sulfonate (6)]).

“Effector functions” refer to those biological activities attributable to the Fc-region of an antibody, which vary with the antibody class. Such an Fc-region is denoted as “effector function competent” herein. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 10³-fold and eliminated the human monocyte response to antibody-sensitized red blood cells (Armour, K. L., et al., Eur. J. Immunol. 29 (1999) 2613-2624).
FcγRII (CD32) binds complexed IgG with medium to low affinity and is widely expressed. This receptor can be divided into two sub-types, FcγRIIA and FcγRIIB FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

The term “Fc receptor” as used herein refers to activation receptors characterized by the presence of a cytoplasmatic ITAM sequence associated with the receptor (see e.g. Ravetch, J. V. and Bolland, S., Annu. Rev. Immunol. 19 (2001) 275-290). Such receptors are FcγRI, FcγRIIA and FcγRIIIA The term “no binding of FcγR” denotes that at an antibody concentration of 10 μg/ml the binding of an antibody as reported herein to NK cells is 10% or less of the binding found for anti-OX40L antibody LC.001 as reported in WO 2006/029879.

While IgG4 shows reduced FcR binding, antibodies of other IgG subclasses show strong binding. However, Pro238, Asp265, Asp270, Asn297 (loss of Fc carbohydrate), Pro329 and 234, 235, 236 and 237, Ile253, Ser254, Lys288, Thr307, Gln311, Asn434, and His435 are residues which provide if altered also reduce FcR binding (Shields, R. L., et al. J. Biol. Chem. 276 (2001) 6591-6604; Lund, J., et al., FASEB J. 9 (1995) 115-119; Morgan, A., et al., Immunology 86 (1995) 319-324; and EP 0 307 434).

The term “Fc-region” herein is used to define a C-terminal region of an immunoglobulin heavy chain that contains at least a portion of the constant region. The term includes native sequence Fc-regions and variant Fc-regions. In one embodiment, a human IgG heavy chain Fc-region extends from Cys226, or from Pro230, to the carboxyl-terminus of the heavy chain. However, the C-terminal lysine (Lys447) of the Fc-region may or may not be present. Unless otherwise specified herein, numbering of amino acid residues in the Fc-region or constant region is according to the EU numbering system, also called the EU index, as described in Kabat, E. A. et al., Sequences of Proteins of Immunological Interest, 5th ed., Public Health Service, National Institutes of Health, Bethesda, Md. (1991), NIH Publication 91-3242.

The antibodies used in the methods as reported herein comprise an Fc-region, in one embodiment an Fc-region derived from human origin. In one embodiment the Fc-region comprises all parts of the human constant region. The Fc-region of an antibody is directly involved in complement activation, C1q binding, C3 activation and Fc receptor binding. While the influence of an antibody on the complement system is dependent on certain conditions, binding to C1q is caused by defined binding sites in the Fc-region. Such binding sites are known in the state of the art and described e.g. by Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such binding sites are e.g. L234, L235, D270, N297, E318, K320, K322, P331 and P329 (numbering according to EU index of Kabat). Antibodies of subclass IgG1, IgG2 and IgG3 usually show complement activation, C1q binding and C3 activation, whereas IgG4 do not activate the complement system, do not bind C1q and do not activate C3. An “Fc-region of an antibody” is a term well known to the skilled artisan and defined on the basis of papain cleavage of antibodies. In one embodiment the Fc-region is a human Fc-region.

The terms “full length antibody”, “intact antibody,” and “whole antibody” are used herein interchangeably to refer to an antibody having a structure substantially similar to a native antibody structure or having heavy chains that contain an Fc-region as defined herein.

An “individual” or “subject” is a mammal. Mammals include, but are not limited to, domesticated animals (e.g. cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman, S. et al., J. Chromatogr. B 848 (2007) 79-87.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3), whereby between the first and the second constant domain a hinge region is located. Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “native effector function” refer to the effector function associated with naturally occurring immunoglobulin molecules with varying structures, i.e. of native antibodies.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “blood-brain barrier” (BBB) denotes the physiological barrier between the peripheral circulation and the brain and spinal cord which is formed by tight junctions within the brain capillary endothelial plasma membranes, creating a tight barrier that restricts the transport of molecules into the brain, even very small molecules such as urea (60 Daltons). The BBB within the brain, the blood-spinal cord barrier within the spinal cord, and the blood-retinal barrier within the retina are contiguous capillary barriers within the CNS, and are herein collectively referred to as the blood-brain barrier or BBB. The BBB also encompasses the blood-CSF barrier (choroid plexus) where the barrier is comprised of ependymal cells rather than capillary endothelial cells.

The term “central nervous system” (CNS) denotes the complex of nerve tissues that control bodily function, and includes the brain and spinal cord.

The term “blood-brain barrier receptor” (BBBR) denotes an extracellular membrane-linked receptor protein expressed on brain endothelial cells which is capable of transporting molecules across the BBB or be used to transport exogenous administrated molecules. Examples of BBBR include but are not limited to transferrin receptor (TfR), especially transferrin receptor 1 (TfR1), insulin receptor, insulin-like growth factor receptor (IGF-R), low density lipoprotein receptors including without limitation low density lipoprotein receptor-related protein 1 (LRP1) and low density lipoprotein receptor-related protein 8 (LRP8), and heparin-binding epidermal growth factor-like growth factor (HB-EGF). An exemplary BBBR is the human transferrin receptor (TfR), especially the transferrin receptor 1 (TfR1).

The term “monovalent binding entity” denotes a molecule able to bind specifically and in a monovalent binding mode to a BBBR. The blood brain shuttle module and/or conjugate as reported herein are characterized by the presence of a single unit of a monovalent binding entity i.e. the blood brain shuttle module and/or conjugate of the present invention comprise exactly one unit of the monovalent binding entity. The monovalent binding entity includes but is not limited to polypeptides, full length antibodies, antibody fragments including Fab, Fab′, Fv fragments, single-chain antibody molecules such as e.g. single chain Fab, scFv. The monovalent binding entity can for example be a scaffold protein engineered using state of the art technologies like phage display or immunization. The monovalent binding entity can also be a polypeptide. In certain embodiments, the monovalent binding entity comprises a CH2-CH3 Ig domain and a single chain Fab (scFab) directed to a blood brain barrier receptor. The scFab is coupled to the C-terminal end of the CH2-CH3 Ig domain by a linker. In certain embodiments, the scFab is directed to human transferrin receptor (transferrin receptor 1).

The term “monovalent binding mode” denotes a specific binding to the BBBR where the interaction between the monovalent binding entity and the BBBR takes place through one single epitope. The monovalent binding mode prevents any dimerization/multimerization of the BBBR due to a single epitope interaction point. The monovalent binding mode prevents that the intracellular sorting of the BBBR is altered.

The term “epitope” denotes any polypeptide determinant capable of specific binding to an antibody. In certain embodiments, epitope determinants include chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody.

The terms “(human) transferrin receptor (TfR)” and “transferrin receptor 1” (TfR1) are used interchangeably herein. They denote a transmembrane glycoprotein (with a molecular weight of about 180,000 Da) which is composed of two disulfide-bonded sub-units (each of apparent molecular weight of about 90,000 Da) and is involved in iron uptake in vertebrates. In one embodiment, the TfR (TfR1) herein is human TfR (TfR1) comprising the amino acid sequence as reported in Schneider et al. (Nature 311 (1984) 675-678).

The term “neurological disorder” denotes a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. For the purposes of this application, the CNS will be understood to include the eye, which is normally sequestered from the rest of the body by the blood-retina barrier. Specific examples of neurological disorders include, but are not limited to, neurodegenerative diseases (including, but not limited to, Lewy body disease, post poliomyelitis syndrome, Shy-Draeger syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g. of the CNS and/or brain, including brain metastases resulting from cancer elsewhere in the body).

The term “neurological disorder drug” denotes a drug or therapeutic agent that treats one or more neurological disorder(s). Neurological disorder drugs include, but are not limited to, small molecule compounds, antibodies, peptides, proteins, natural ligands of one or more CNS target(s), modified versions of natural ligands of one or more CNS target(s), aptamers, inhibitory nucleic acids (i.e., small inhibitory RNAs (siRNA) and short hairpin RNAs (shRNA)), ribozymes, and small molecules, or active fragments of any of the foregoing. Exemplary neurological disorder drugs include, but are not limited to: antibodies, aptamers, proteins, peptides, inhibitory nucleic acids and small molecules and active fragments of any of the foregoing that either are themselves or specifically recognize and/or act upon (i.e., inhibit, activate, or detect) a CNS antigen or target molecule such as, but not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins. Non-limiting examples of neurological disorder drugs and the corresponding disorders they may be used to treat: Brain-derived neurotrophic factor (BDNF), Chronic brain injury (Neurogenesis), Fibroblast growth factor 2 (FGF-2), Anti-Epidermal Growth Factor Receptor Brain cancer, (EGFR)-antibody, glial cell-line derived neural factor Parkinson's disease, (GDNF), Brain-derived neurotrophic factor (BDNF) Amyotrophic lateral sclerosis, depression, Lysosomal enzyme Lysosomal storage disorders of the brain, Ciliary neurotrophic factor (CNTF) Amyotrophic lateral sclerosis, Neuregulin-1 Schizophrenia, Anti-HER2 antibody (e.g. trastuzumab) Brain metastasis from HER2-positive cancer.

The term “imaging agent” denotes a compound that has one or more properties that permit its presence and/or location to be detected directly or indirectly. Examples of such imaging agents include proteins and small molecule compounds incorporating a labeled entity that permits detection.

The terms “CNS antigen” and “brain target” denote an antigen and/or molecule expressed in the CNS, including the brain, which can be targeted with an antibody or small molecule. Examples of such antigen and/or molecule include, without limitation: beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6. In one embodiment, the antigen is BACE1.

The term “that specifically binds” denotes an antibody selectively or preferentially binding to an antigen. The binding affinity is generally determined using a standard assay, such as Scatchard analysis, or surface plasmon resonance technique (e.g. using BIACORE®).

A “conjugate” is a fusion protein conjugated to one or more heterologous molecule(s), including but not limited to a label, neurological disorder drug or cytotoxic agent.

The term “linker” denotes a chemical linker or a single chain peptidic linker that covalently connects different entities of the blood brain barrier shuttle module and/or the fusion polypeptide and/or the conjugate as reported herein. The linker connects for example the brain effector entity to the monovalent binding entity. For example, if the monovalent binding entity comprises a CH2-CH3 Ig entity and a scFab directed to the blood brain barrier receptor, then the linker conjugates the scFab to the C-terminal end of the CH3-CH2 Ig entity. The linker conjugating the brain effector entity to the monovalent binding entity (first linker) and the linker connecting the scFab to the C-terminal end of the CH2-CH3 Ig domain (second linker) can be the same or different.

Single chain peptidic linkers, comprising of from one to twenty amino acid residues joined by peptide bonds, can be used. In certain embodiments, the amino acids are selected from the twenty naturally-occurring amino acids. In certain other embodiments, one or more of the amino acids are selected from glycine, alanine, proline, asparagine, glutamine and lysine. In other embodiments, the linker is a chemical linker. In certain embodiments, the linker is a single chain peptidic linker with an amino acid sequence with a length of at least 25 amino acid residues, in one preferred embodiment with a length of 32 to 50 amino acid residues. In one embodiment the peptidic linker is a (GxS)n linker with G=glycine, S=serine, (x=3, n=8, 9 or 10) or (x=4 and n=6, 7 or 8), in one embodiment with x=4, n=6 or 7, in one preferred embodiment with x=4, n=7. In one embodiment the linker is (G4S)4 (SEQ ID NO: 37). In one embodiment the linker is (G4S)6G2 (SEQ ID NO: 38).

Conjugation may be performed using a variety of chemical linkers. For example, the monovalent binding entity or the fusion polypeptide and the brain effector entity may be conjugated using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). The linker may be a “cleavable linker” facilitating release of the effector entity upon delivery to the brain. For example, an acid-labile linker, peptidase-sensitive linker, photolabile linker, dimethyl linker or disulfide-containing linker (Chari et al, Cancer Res. 52 (1992) 127-131; U.S. Pat. No. 5,208,020) may be used.

Covalent conjugation can either be direct or via a linker. In certain embodiments, direct conjugation is by construction of a polypeptide fusion (i.e. by genetic fusion of the two genes encoding the monovalent binding entity towards the BBBR and effector entity and expressed as a single polypeptide (chain)). In certain embodiments, direct conjugation is by formation of a covalent bond between a reactive group on one of the two portions of the monovalent binding entity against the BBBR and a corresponding group or acceptor on the brain effector entity. In certain embodiments, direct conjugation is by modification (i.e. genetic modification) of one of the two molecules to be conjugated to include a reactive group (as non-limiting examples, a sulfhydryl group or a carboxyl group) that forms a covalent attachment to the other molecule to be conjugated under appropriate conditions. As one non-limiting example, a molecule (i.e. an amino acid) with a desired reactive group (i.e. a cysteine residue) may be introduced into, e.g., the monovalent binding entity towards the BBBR antibody and a disulfide bond formed with the neurological drug. Methods for covalent conjugation of nucleic acids to proteins are also known in the art (i.e., photocrosslinking, see, e.g., Zatsepin et al. Russ. Chem. Rev. 74 (2005) 77-95). Conjugation may also be performed using a variety of linkers. For example, a monovalent binding entity and a effector entity may be conjugated using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Peptidic linkers, comprised of from one to twenty amino acid residues joined by peptide bonds, may also be used. In certain such embodiments, the amino acid residues are selected from the twenty naturally-occurring amino acids. In certain other such embodiments, one or more of the amino acid residues are selected from glycine, alanine, proline, asparagine, glutamine and lysine. The linker may be a “cleavable linker” facilitating release of the effector entity upon delivery to the brain. For example, an acid-labile linker, peptidase-sensitive linker, photolabile linker, dimethyl linker or disulfide-containing linker (Chari et al, Cancer Res. 52 (1992) 127-131; U.S. Pat. No. 5,208,020) may be used.

The term “infusion-related side-effect” refers to an unintended adverse event associated with the treatment of a subject with a therapeutic antibody. In one embodiment this infusion-related side effect is selected from the group consisting of vasodilation, bronchoconstriction, laryngeal edema, drop of cardiac pressure and hypothermia (after intravenous application). In one embodiment such an event is hypothermia resulting in a drop of the body-temperature within two hours after i.v. administration of the therapeutic antibody.

The term “effector cell” refers to an immune cell which is involved in the effector phase of an immune response. Exemplary immune cells include a cell of a myeloid or lymphoid origin, for instance lymphocytes (such as B-cells and T-cells including cytolytic T cells (CTLs)), killer cells, natural killer cells, macrophages, monocytes, eosinophils, neutrophils, polymorphonuclear cells, granulocytes, mast cells, and basophiles. Some effector cells express specific Fc-receptors and carry out specific immune functions. In some embodiments, an effector cell is capable of inducing antibody-dependent cellular cytotoxicity (ADCC), such as a neutrophil capable of inducing ADCC. For example, monocytes, macrophages, which express Fc-receptors are involved in specific killing of target cells and presenting antigens to other components of the immune system, or binding to cells that present antigens.

As described herein below, the term “reduced side effect after administration” as used herein is relative to the side effect after administration that a fully effector-functional mAb has (i.e. an antibody having full effector-function that is not sterically or otherwise hindered). In particular and for practical reasons, the reduced side-effect of the bispecific antibody of the present invention may be determined relative to the same antibody but which lacks the two binding sites specifically binding to a first (cell surface) target, particularly which lacks the two Fab parts of the antibody directed against the first target. Such a construct to which the antibody of the present invention is compared is e.g. shown herein in Table 1 as “mBS-noFab”.

### Compositions and Methods

Antibodies and antibody fragments against the transferrin receptor (TfR1) have been used to transport large molecules into the brain by receptor-mediated transcytosis (Yu et al., 2011; Niewoehner et al., 2014). However, a recent study has revealed a liability previously overlooked using conventional mAbs against TfR1 (Couch et al., 2013). Acute clinical signs were observed in mice directly after dosing and this was linked to the effector function status of the mAb. This was also observed when using bispecific mAbs where only one Fab arm binds to TfR1, providing the mAb contained a native fully active effector function. Taken together, the effector function of the antibody is directly linked to the observed acute clinical signs, and so an obvious strategy would be to use an effector-dead variant. However, for certain mAbs a native effector function is crucial for the mode-of-action and optimal therapeutic profile. Through mAb engineering and careful in vitro and in vivo assessments in a novel FcγR-humanized mouse model multiple constructs of the BS-mAb system (fusion of a brain-shuttle module, i.e. a monovalent anti-TfR1 binding site, to the Fc-region at the C-terminal end of a heavy or light chain of a therapeutic monoclonal antibody resulting in a Brain Shuttle-mAb (BS-mAb)) were compared to assess the potential AIR (acute infusion reaction) liability of using native IgG effector function.

The current invention is based at least in part on the finding that the effector function of a TfR1-targeting BS-mAb is masked when binding to TfR1 but is back to an active configuration when it binds its CNS target. Without being bound by this theory this dual behavior can be ascribed to steric hindrance of the binding of the Fc-region with the FcγR on immune cells when TfR1 is bound by the BS Fab/BS-mAb. In this position the two Fab arms at the opposite, N-terminal end of the BS-mAb prevent the necessary proximity of the Fc-region of the BS-mAb to the FcγR on effector cells. Once the BS-mAb is released from the TfR1 into the CNS parenchyma and the resident target is bound by the N-terminal Fabs, the free BS-Fab on the C-terminal end does not longer interfere with the interaction with FcγR on resident effector cells. Thus, these data provide the basis for the use of fully effector-functional mAbs that can be transported safely across the BBB.

The invention is at least in part based on the finding that the Fc-region effector function of TfR1-targeting BS-mAbs is camouflaged when the mAb binds to TfR1 but is back to an active configuration when the mAb binds its CNS target.

The invention is at least in part based on the finding that both therapeutic target binding Fabs arms are required to maximize the inhibitory effect on FcγR recruitment in order to minimize infusion-related drop of the body-temperature and cytokine release.

Thus, the effect as reported herein is linked to the bispecific, trivalent format of the BS-mAb, i.e. a full length bivalent, monospecific antibody which is conjugated at one of its heavy chain C-termini to a BS-Fab. This shielding effect is not observed with a conventional bivalent, bispecific antibody.

The method as reported herein is exemplified in the following with a Brain Shuttle-monoclonal antibody (BS-mAb) specifically binding to amyloid-β fibrils/plaques as therapeutic target and to human transferrin receptor 1 as BBB shuttle receptor, denoted as BS-mAb31. MAb31 is an anti-Aβ mAb which specifically recognizes oligomeric and fibril structure with a high apparent affinity for Aβ plaques (14). All constructs used contained a human native IgG1 Fc-region with full effector function, except for the effector-dead (P329G/L234A/L235A) mutation variants. These constructs are simply used to exemplify the current invention and shall not be construed as limitation of the scope of the invention, which is set forth in the claims.

The BS module is fused to the Fc-region at the C-terminal end of a heavy or light chain of a conventional therapeutic mAb resulting in a Brain Shuttle-mAb (BS-mAb). This preserves the natural configuration of the BS-mAb with two different configurations either binding to the target for therapeutic effects or binding to the TfR1 for BBB transport.

### Experimental Results

Brain Shuttle-mAb Maintains Fc-Region Effector Function in Free Form and Engaged with Therapeutic Antigen Direct-Target.

Fc-region effector function is responsible for antibody-dependent cell-mediated cytotoxicity (ADCC) and/or complement-dependent cytotoxicity (CDC). The FcγR binding of the BS-mAb31 construct and the mAb31 IgG counterpart were confirmed as outlined in the following.

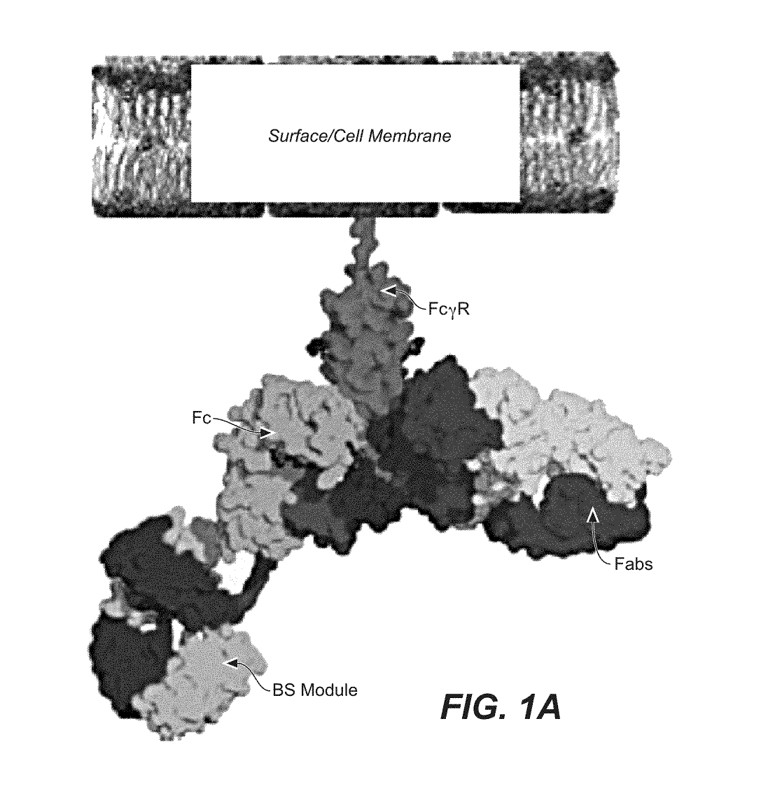

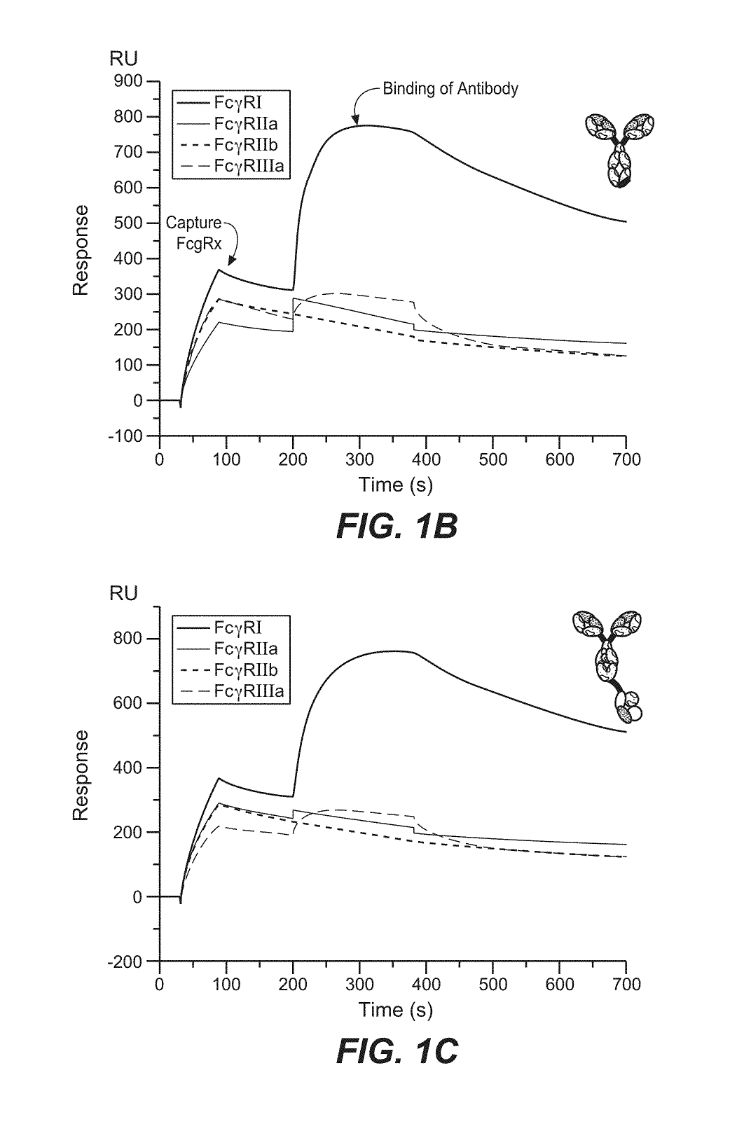

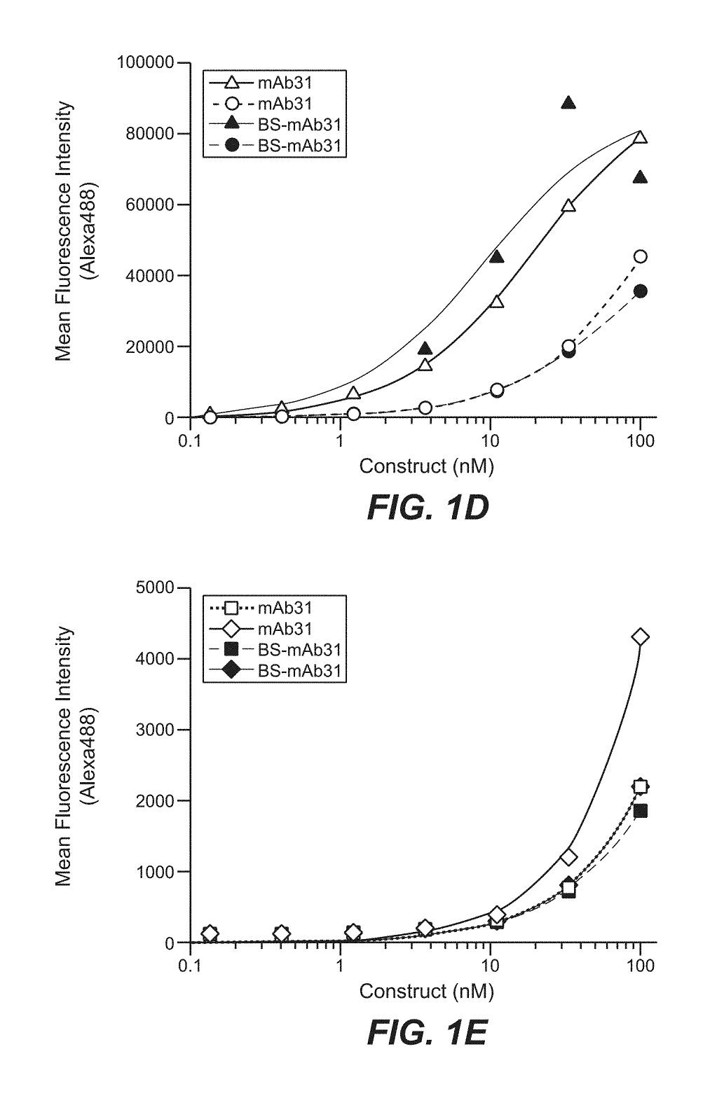

The first binding studies were performed with the antibody (BS-mAb31 and the parental mAb31) in solution and the FcγR immobilized on a 2-dimensional surface. This allows determining the interaction between the mAb free in solution and the immobilized FcγR in any orientation (FIG. 1A). First, a surface plasmon resonance (SPR) based assay was used wherein four different FcγR were immobilized in the flow channels as the immobilized target. The SPR results showed that both constructs bind the different FcγRs similar and according to low and high affinity receptors (see FIGS. 1B and 1C). The binding profile from the SPR experiments are in agreement with reported rank-order in binding affinities against the different FcγRs (13).

Second, cell binding experiments were performed wherein the different human FcγRs were individually overexpressed on a cell. Both the BS-mAb31 and the parental mAb31 bound equally well (FIGS. 1D and 1E) and the rank-order was in agreement with the SPR data.

Taken together, there were no differences in the Fc-region-FcγRs interaction for these two constructs (BS-mAb and parental mAb), when the receptors are presented in a non-constrained fashion (either on SPR surface or on the more native cell surface). This shows that the Fc-region area engaged in the FcγR interaction is fully functional, even when a BS-module is fused to the C-terminal end of the Fc-region.

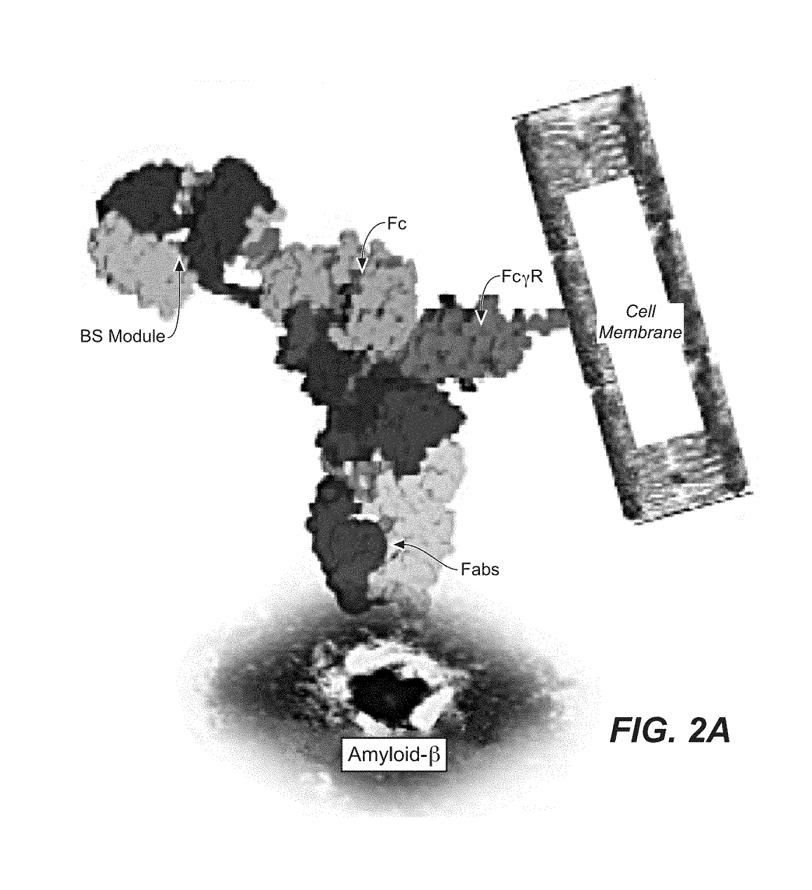

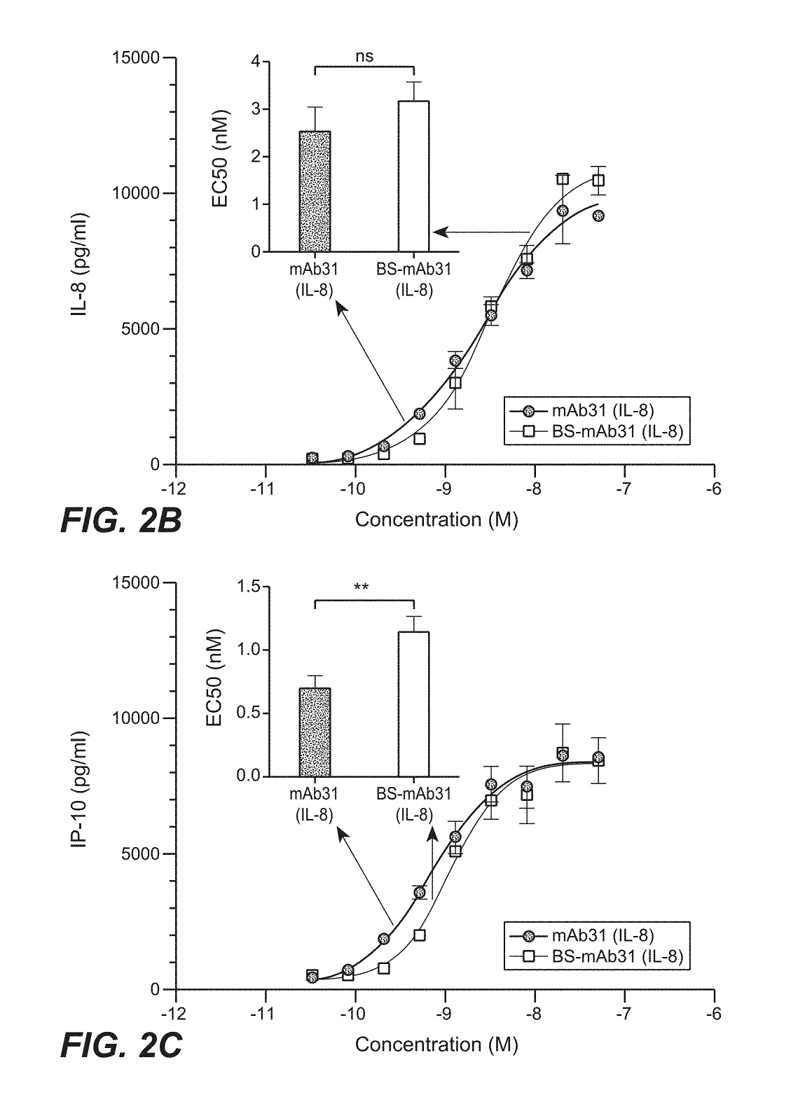

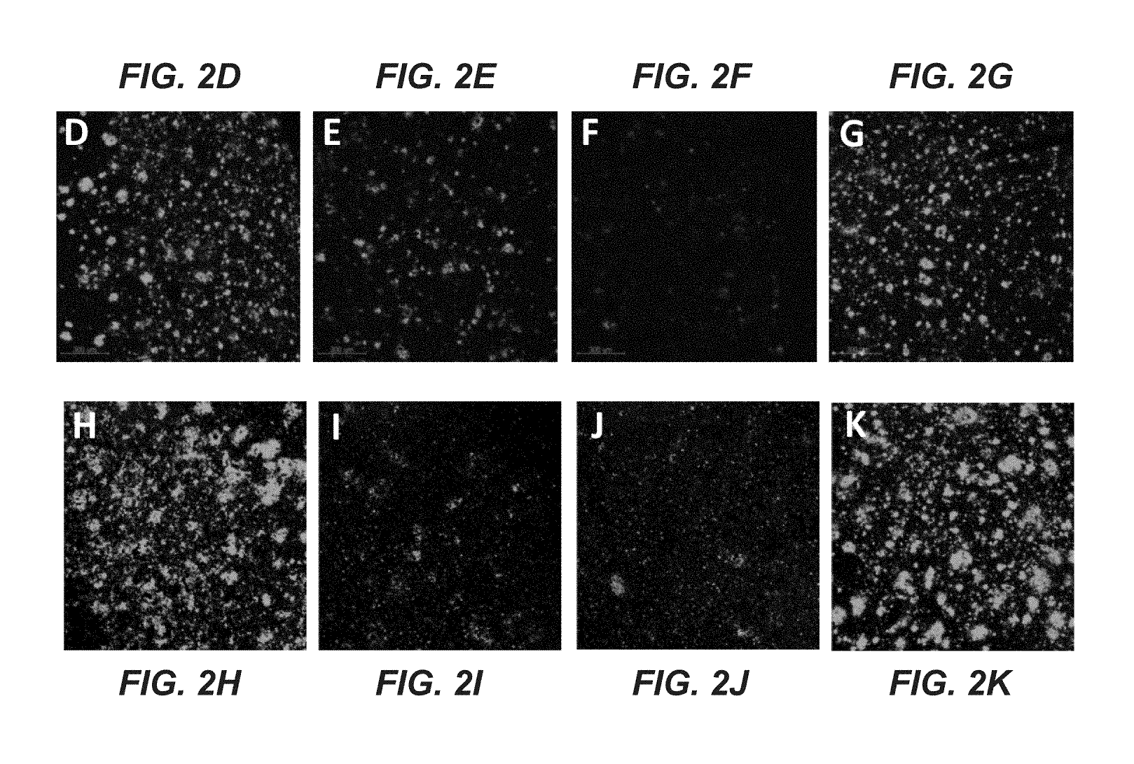

Next it was investigated if the BS-mAb31 construct maintains Fc-region effector function when the antibody is interacting with its therapeutic target. Binding to its target will present the constructs for FcγR interaction in a more defined, inflexible, with less steric hindrance, more native-like conformation compared to the situation free in solution (FIG. 2A). Therefore, an antibody-dependent cellular cytotoxicity (ADCC) assay employing human Aβ protein coated on a surface and a monocytic cell was used to simulate an effector cell presenting FcγRs. Two different cytokines were used as readout for cytotoxicity. It was found that BS-mAb31 had a potency comparable to parental mAb31 (see FIGS. 2B and 2C). Without being bound by this theory the Brain Shuttle construct, when bound to its therapeutic target, presents the Fc-region in an orientation that prevents interference from the BS module.

In a second assay, a phagocytosis assay, postmortem Alzheimer's disease (AD) brain tissue slices cultured with primary human effector cells were employed (14). AD brain sections were pre-incubated with different concentrations of BS-mAb31 and parental mAb31 followed by incubation with effector cells. A concentration-dependent decrease of Aβ plaque load was observed (see FIGS. 2D and 2K) for both antibodies. These results are in line with the art confirming that Fc-receptor/microglia-mediated phagocytosis of mAb-decorated Aβ plaques is a major mechanism of Aβ plaque clearance in the brain (15-17). Without being bound by this theory it can be concluded that FcγR engagement and microglia recruitment is not hampered by the fused BS-module when the Brain Shuttle construct engages with its therapeutic target on the cell's surface or decorates the Aβ plaques in the brain.

The Brain Shuttle Improves In Vivo Efficacy in Brain of mAb31 Despite Faster Plasma Clearance.

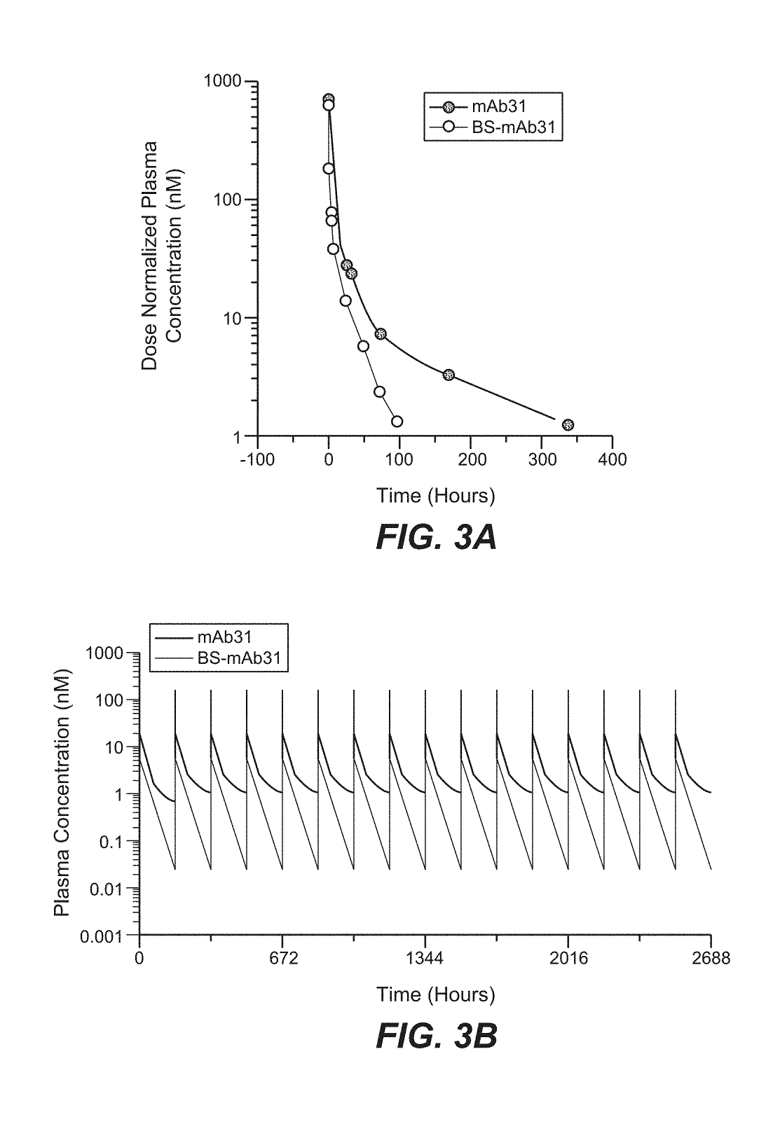

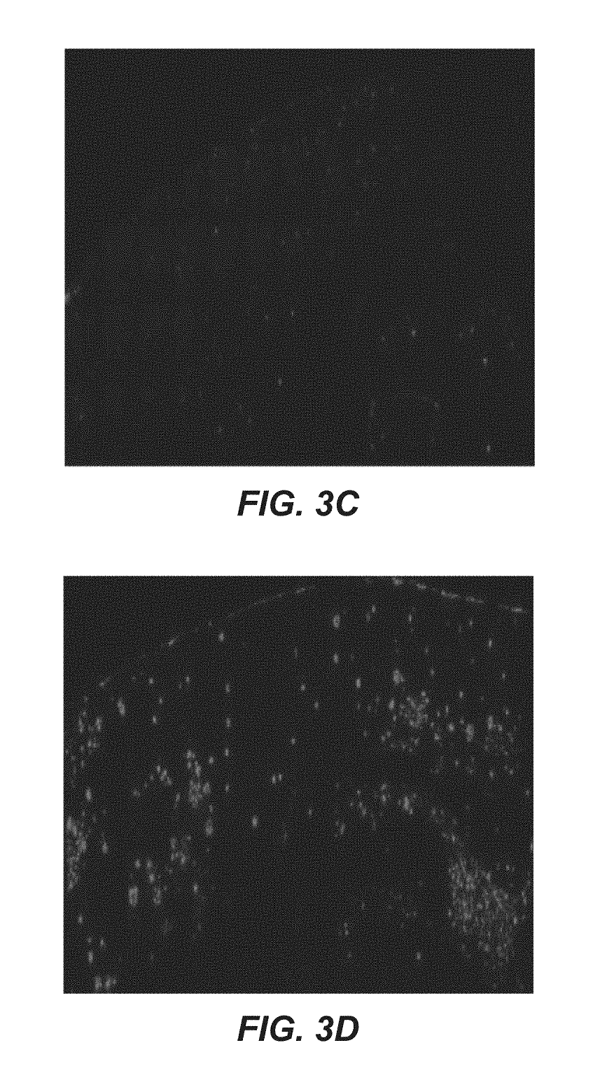

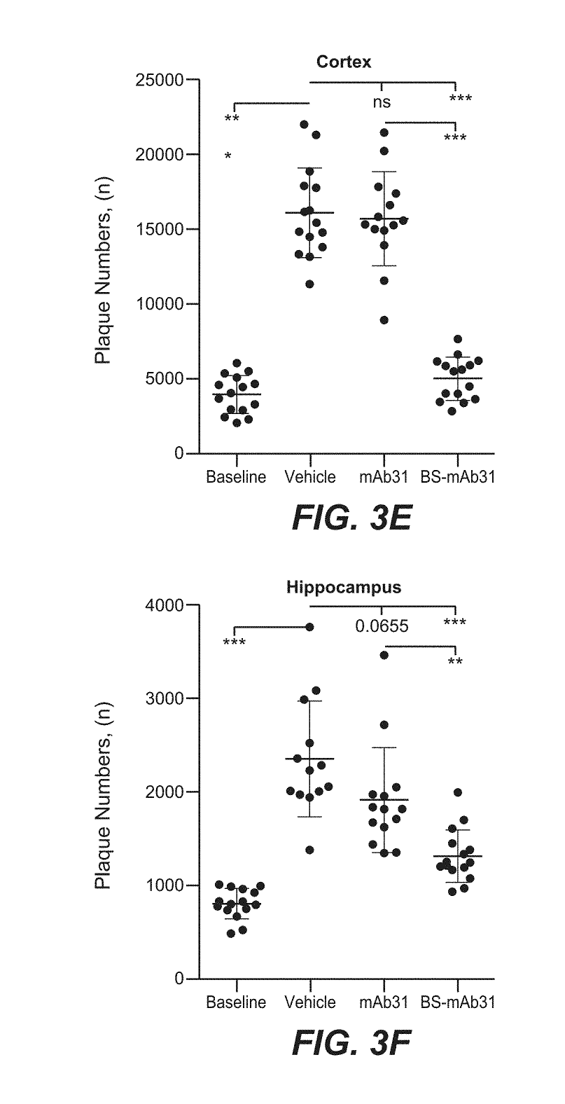

To translate the in vitro plaque clearance effects in an appropriate in vivo model, plaque reduction properties of the BS-mAb31 construct versus the parental mAb31 were investigated in a transgenic amyloidosis mouse model (APP London: APP V717I) (18). The plasma exposure was lower for the BS-mAb31 compared to mAb31 (see FIG. 3A). Without being bound by this theory it is assumed that the lower exposure of the BS construct is attributed to target-mediated drug deposition (TMDD) through binding to TfR1 in the periphery.

A 4-month efficacy study was designed based on weekly dosing and the plasma exposure was simulated (see FIG. 3B). A much higher and persistence exposure was predicted for the parental mAb31. However, previous data has also shown that brain exposure of the BS-mAb31 is considerably greater than the parental mAb31 in the PS2APP transgenic mouse model (9).

Target engagement in the cortex of the anti-Aβ mAb and its Brain Shuttle construct after 4-months of dosing every week was investigated. It was substantially much more plaque decoration detectable with the BS-mAb31 (FIGS. 2C and 2D). In this 4-month chronic treatment study a significant reduction of Aβ amyloid plaques in cortex and hippocampus was visible in BS-mAb31 treated mice compared with vehicle controls and equimolar low-dose of mAb31 even though plasma exposure for the Brain Shuttle was substantial lower (see FIGS. 2E and 2F). The improved efficacy has previously been shown in another amyloidosis transgenic mouse model, where it was demonstrated that a monovalent TfR1 engagement is absolutely essential (9).

Taken together, this data shows that the attached BS-module at the C-terminus of the mAb31 does not interfere with the interaction with the FcγR on microglia cells. Thereby Aβ plaque clearance is promoted besides significantly improved in vivo efficacy by enhanced brain exposure of the therapeutic IgG (9).

The Unique TfR1 Binding Mode of the Brain Shuttle Attenuates the Engagement with FcγRs.

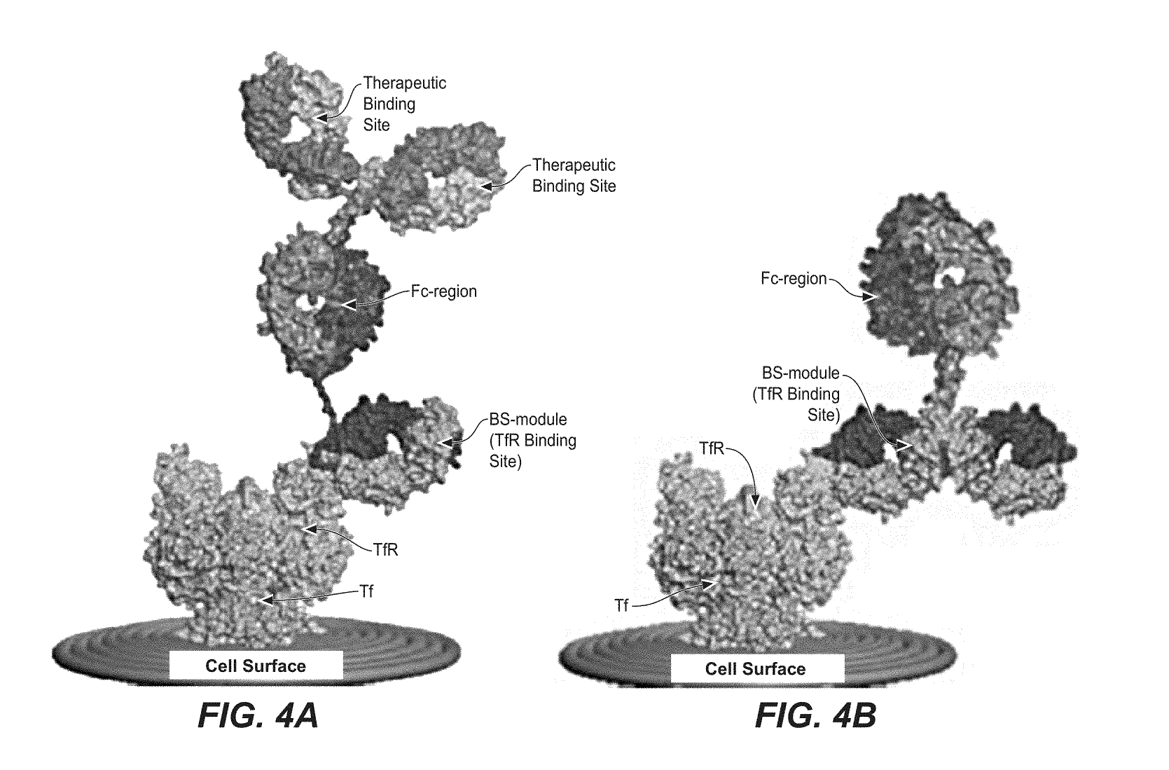

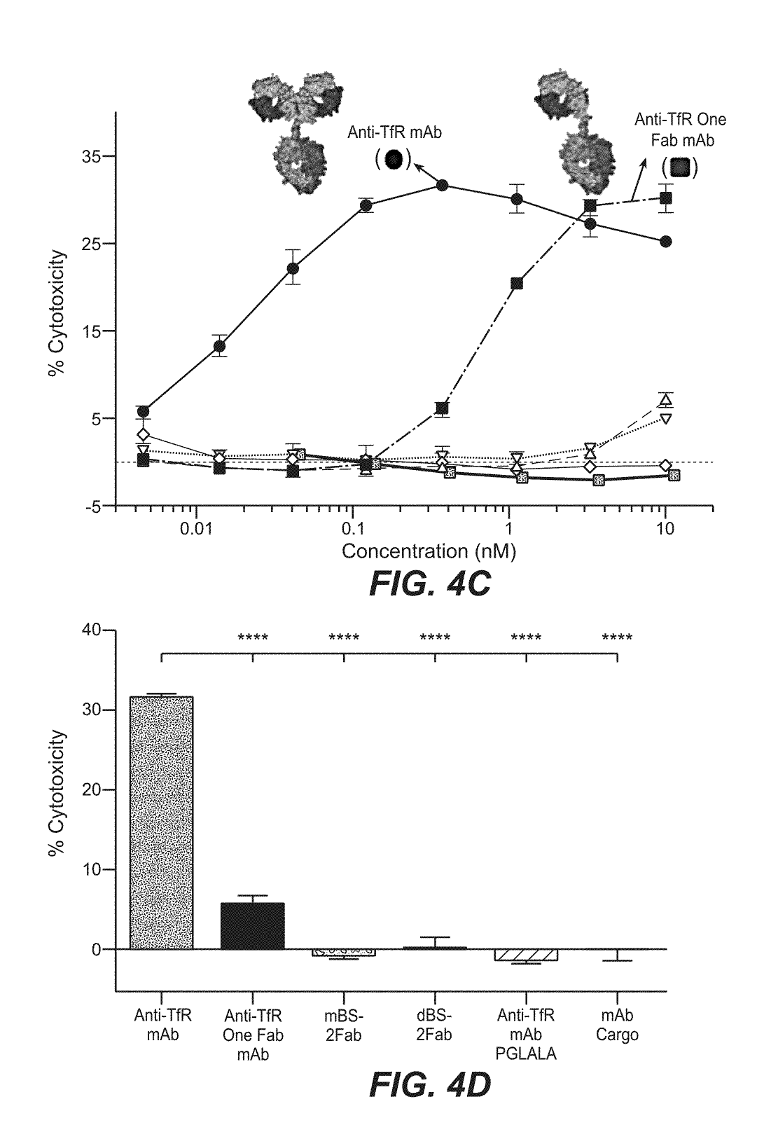

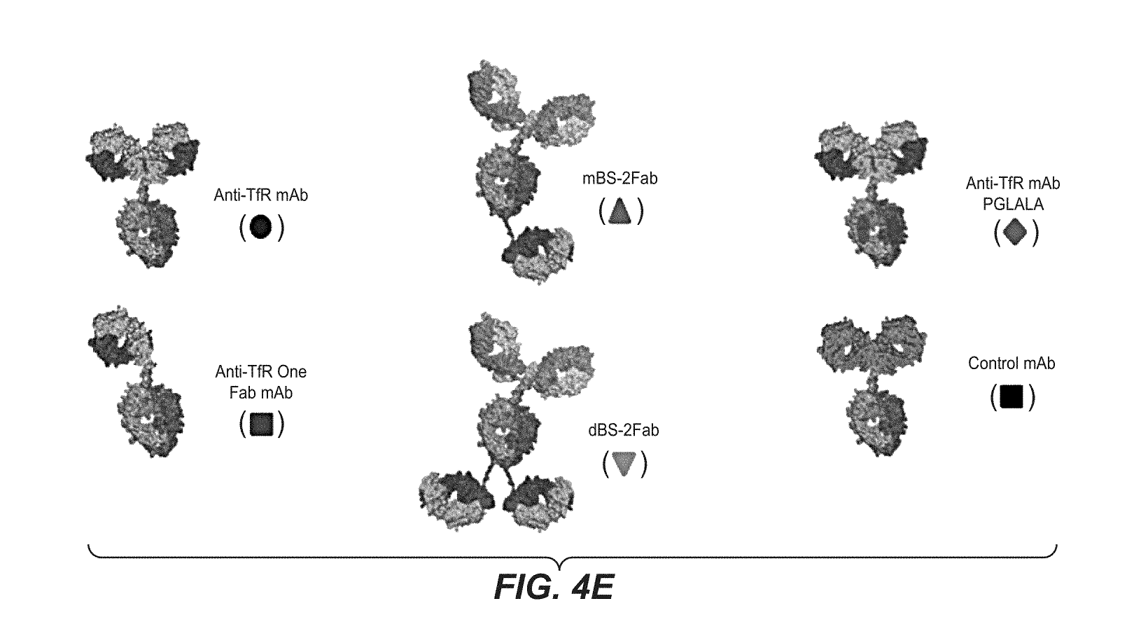

The in vitro TfR1 binding properties of the BS-mAb31 and the anti-TfR1 mAb were investigated. The BS-mAb31 construct contains an anti-TfR1 Fab as the C-terminal BS module. It has been found that the binding to TfR1 of the BS-mAb31 (FIG. 4A) and the bivalent native anti-TfR1 mAb (FIG. 4B) is different resulting in a different spatial presentation of the therapeutic entity (IgG) and the Fc-region towards the environment.

The functionality of the Fc-region when the construct is bound to the TfR1 was determined using an antibody-dependent cell-mediated cytotoxicity (ADCC) assay. In this assay one cell expresses the TfR1 and the other cell (human NK92) has the function of an effector cell expressing FcγRIIIA ADCC is a mechanism of cell-mediated immune defense whereby an effector cell of the immune system actively lyses a target cell, whose membrane-surface antigens have been bound by specific antibodies.

The interaction has been analyzed using three different IgG constructs. As expected the standard anti-TfR1 mAb (bivalent, monospecific) with full effector function produced a strong ADCC response. The anti-TfR1 one Fab mAb also produced an ADCC response but at a higher concentration due to loss of avid binding (FIG. 4C). All cytotoxicity effect was mediated by the Fc-region. Confirmation was done using an anti-TfR1 mAb with no effector function (P329G/L234A/L235A mutation in the Fc-region). This antibody had no effect in this ADCC assay. Interestingly, the two Brain Shuttle constructs with one or two BS modules fused to the C-terminus of the heavy chains of mAb31, had none or very low level of cytotoxicity (FIG. 4C). At the concentration of the standard anti-TfR1 mAb, which provoked the highest ADCC effect, only a small effect was detected for the anti-TfR1 one Fab mAb, whereas all other constructs did not have a detectable effect (FIG. 4D).

It has to be pointed out that for the dBS-2Fab format an inferior brain-shuttling activity had been found previously (9).

These result cannot be explained by the difference in binding strength between the different constructs, as it has previously been shown that a dBS-2Fab construct has a similar apparent TfR1 binding affinity as the anti-TfR1 mAb construct, as both constructs have two Fab binding TfR1 domains (9).

Thus, it has been found that the Fc-region in the Brain shuttle constructs, even though with full effector function, was unable to productively engage with certain FcγRs to induce an ADCC response.

Conventional Anti-TfR1 mAb with Effector Function Causes First Infusion Reactions and Cytokine Inductions.

As shown above the BS-mAb construct maintains its effector function when engaged with its target in the brain. Now it was determined what consequences effector function will have when the BS-antibody binds to the TfR1 through the BS-module. This is important especially in the light of the recent findings that standard Y-shaped anti-TfR1 mAb treatment in mice causes acute clinical signs (12).

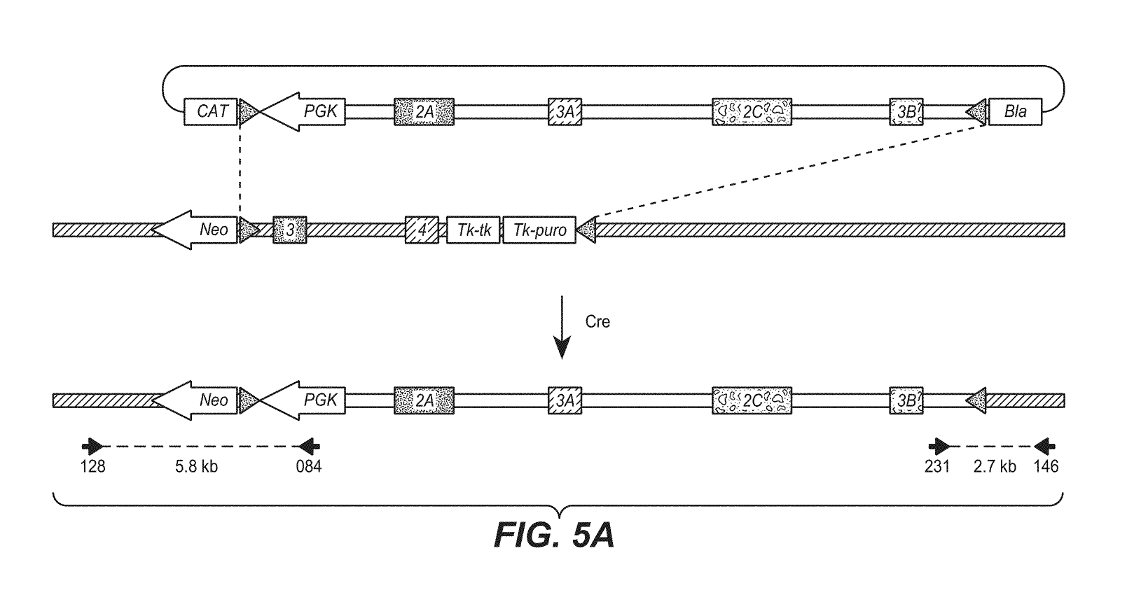

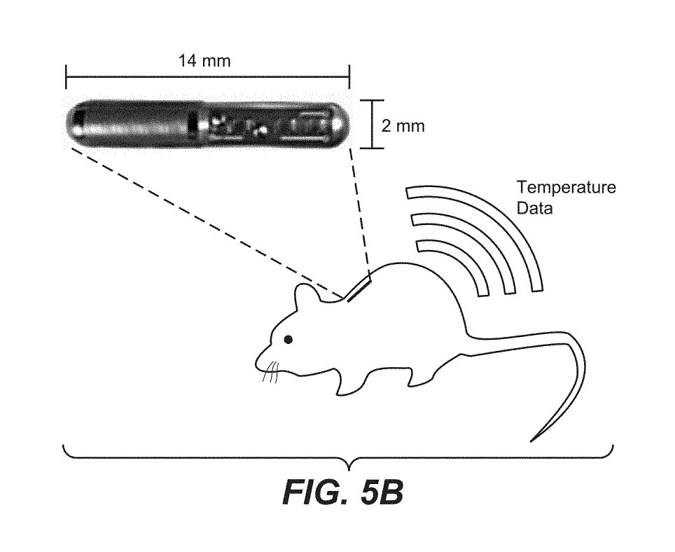

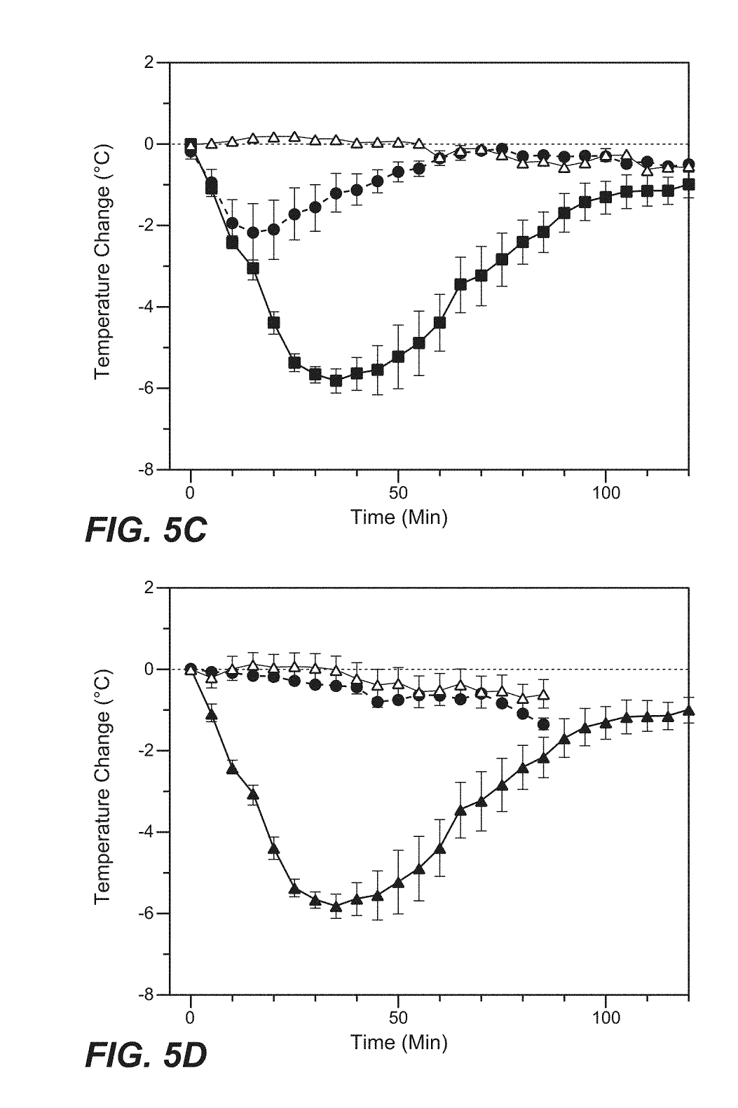

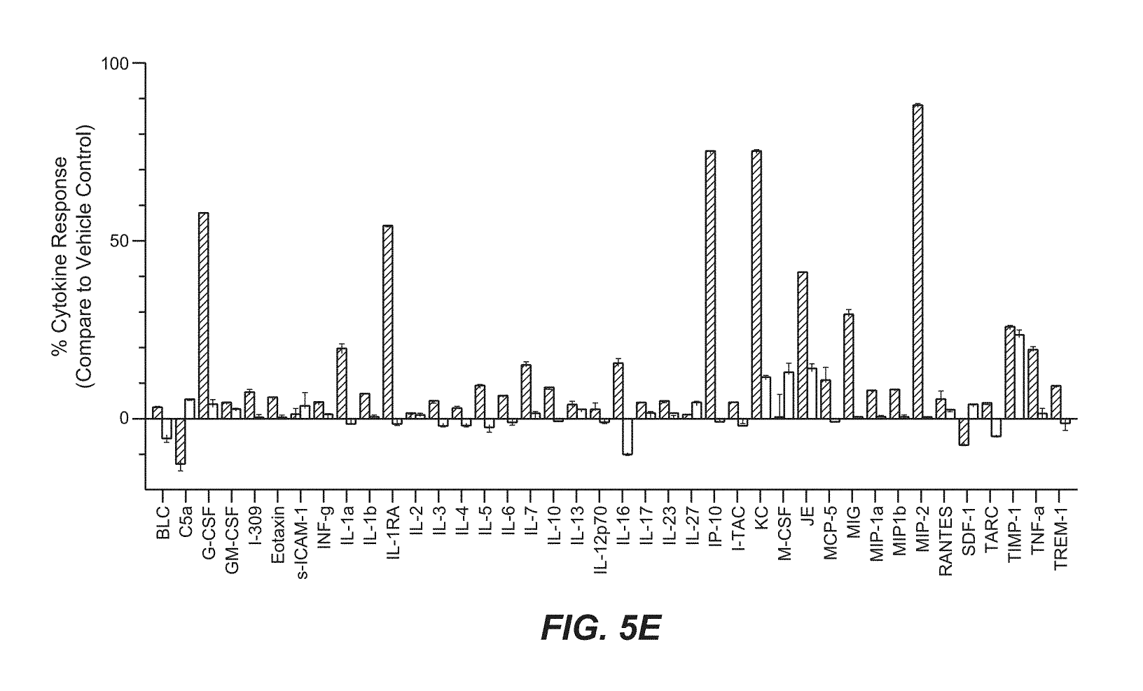

In the first step this was examined in a huFcγR transgenic mouse system. In short, this model was generated through gene-targeted replacement of the two activating low-affinity mouse FcγR genes (Fcγr3 and Fcγr4) by the four human counterparts (FCGR2A, FCGR3A, FCGR2C and FCGR3B) (FIG. 5A). This provides an adequate system to evaluate in vivo the potential interaction between human/humanized mAbs and human FcγRs resulting in the triggering of effector functions. The model uses telemetric temperature readout (FIG. 5B) to monitoring first infusion reactions (FIR). As outlined already above the FIR is induced by the interaction with FcγR and recruitment of effector immune cells. The wireless recording system in this model allows the animals to move freely during the study.

First, the FIR as induced by the injection of a conventional anti-TfR1 mAb was determined. As shown in FIG. 5C the injection of the conventional anti-TfR1 mAb resulted in a concentration-dependent and transient decrease in body temperature, which returned to normal levels within approximately two hours.

Second, the FIR as induced by the injection of a monovalent form of a conventional anti-TfR1 mAb was determined. The monovalent form of a conventional anti-TfR1 mAb contains only one Fab arm against TfR1. Also this mAb strongly induced FIR. Thus, it has been found hereby that dimerization/multimerization of the TfR1 through bivalent mAb binding is not responsible for the temperature drop.

Third, the relative contribution of effector function to the FIR observed in this model with anti-TfR1 mAb was determined using mAbs with mutations in the Fc-region at residues that are required for FcγR binding (20). The Fc-region triple mutant P329G/L234A/L235A, which lacks FcγR interaction, showed no drop in temperature in the model (FIG. 5D). Thus, it has been found that the Fc-region is responsible for the pronounced FIR. This is corroborated by the in vitro data using the Fc-region effector function eliminated construct (FIG. 4C).

The levels of different cytokines as a response of administration of the anti-TfR1 mAb were determined. It was found that certain cell signaling molecules strongly increased in concentration (FIG. 5E). In particular, Granulocyte-colony stimulating factor (G-CSF), keratinocyte-derived cytokine (KC), Macrophage Inflammatory Protein (MIP-2) and Interferon gamma-induced protein 10 (IP-10) showed a strong response. These cytokine responses can be correlated amongst other things to neutrophil activation. As seen in the temperature readout experiments, virtually no effect on cytokine induction was produced when using the IgG construct with eliminated Fc-region effector function (FIG. 5E).

Thus, it has been found in vitro and in vivo that IgG binding to TfR1 present the Fc-region in an accessible position to effector cells in the periphery and can provoke an adaptive immune response.

### Brain Shuttle Binding-Mode to TfR1 Silenced the Effector Functions and Attenuate First Infusion Reactions and Cytokine Production.

The data above demonstrate that the BS-module does not impair the mAb effector function when bound to its therapeutic target (FIGS. 2 and 3). On the other hand, it has been found that conventional mAbs binding to TfR1 can induce FIR via Fc-region-mediated effector functions (FIGS. 4 and 5).

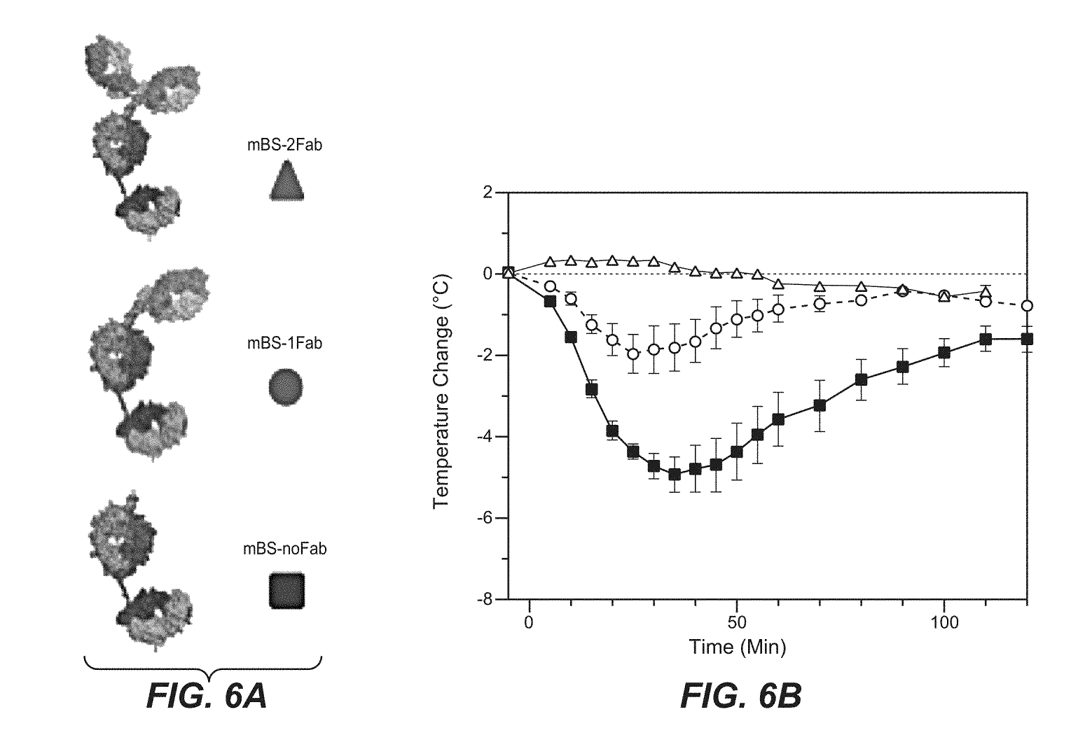

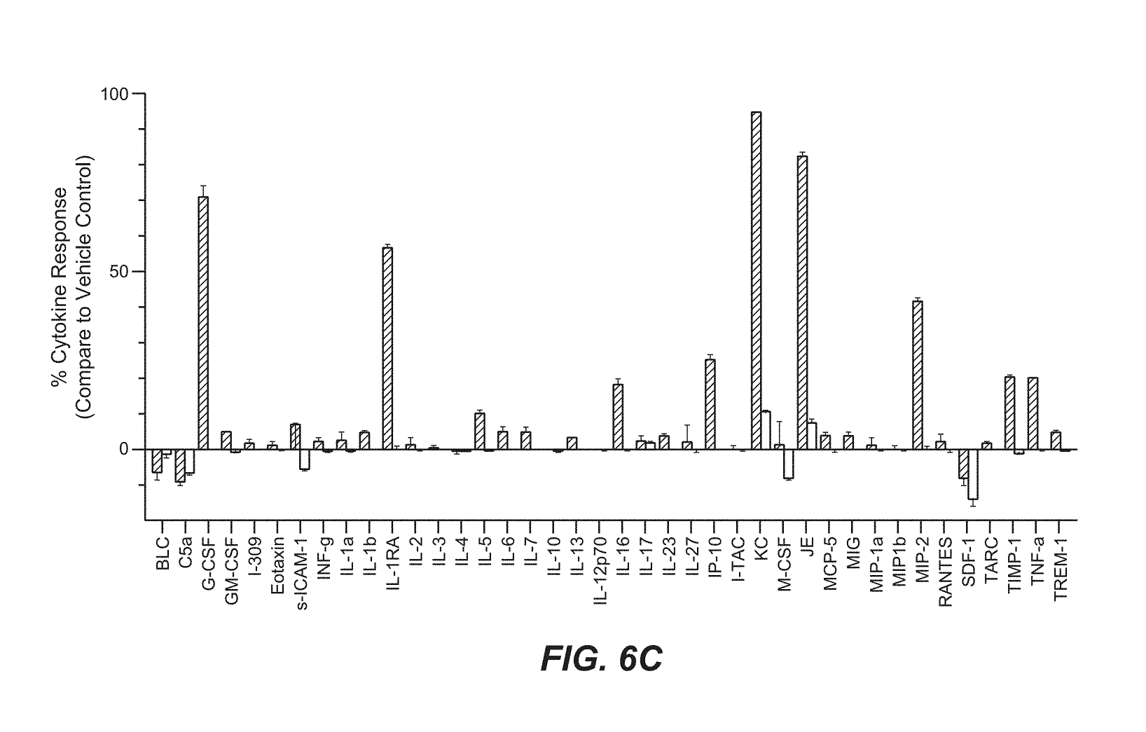

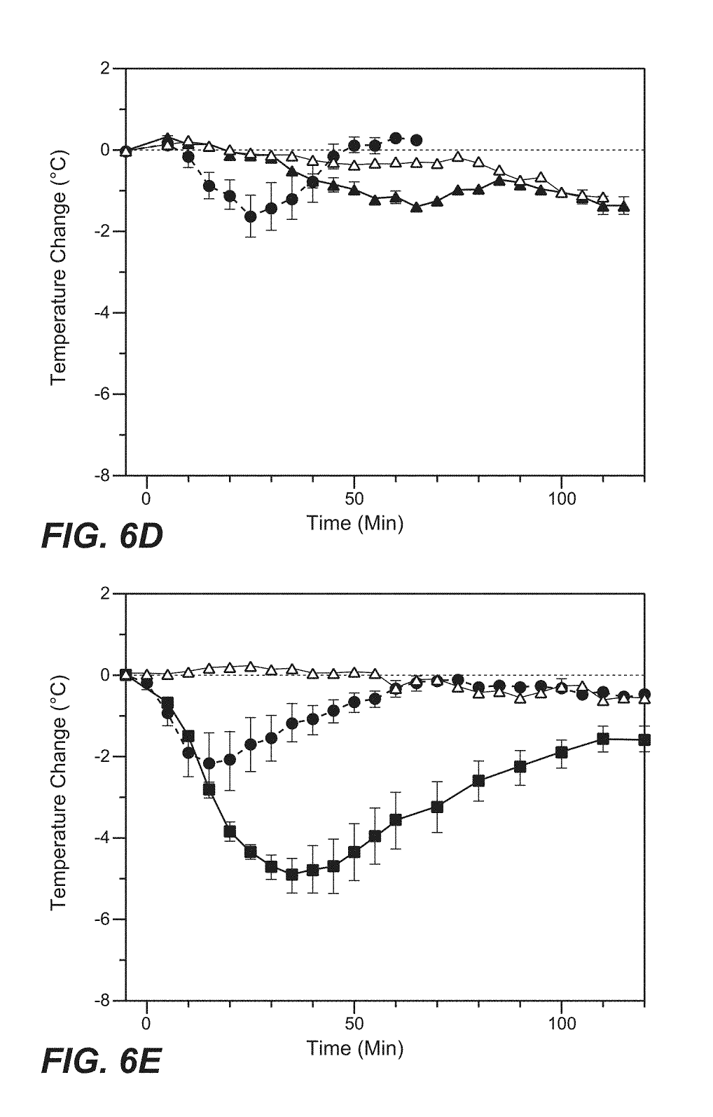

As the TfR1 is widely expressed on peripheral cells the consequences of TfR1 binding through BS-module to these cell types was determined. To assess this, three different BS-mAb constructs were administered to huFcγR transgenic mice (FIG. 6A). These all had human native IgG1 effector function but differed in the number of therapeutically effective Fabs (=binding sites).

Unexpectedly it has been found that no FIR was observed for the standard BS-mAb (denoted as mBS-2Fab in the Figures) construct showing that BS-mAb does not trigger FcγR activation in the periphery in vivo (FIG. 6B).

Without being bound by this theory the following is assumed: When the BS-mAb binds to the TfR1 through the BS module the BS-mAb is presented on the cell surface in a configuration inappropriate for Fc-region recognition (FIG. 4A). The mAb portion of the construct is presented in a reverse orientation with the two Fab arms extending out from the cell surface. In such a configuration the Fc-region of the bound BS-mAb is placed in an inverted orientation in relation to FcγRs on adjacent effector cells. It can be hypothesized that either the inverted orientation of the Fc-region with respect to FcγR or the therapeutic target binding Fab arms extending away from the cell surface play a role in the abrogation of FcγR interaction and the silencing of FIR observed with the BS-mAb construct.

Two constructs with one or both therapeutic target binding Fab arm(s) missing on the mAb portion were designed (FIG. 6A). These constructs, when applied to the huFcγR transgenic mice, clearly caused FIR, as scored by the rapid and strong temperature drop (FIG. 6B). The temperature drop was even more pronounced for the construct lacking both Fab arms (BS-noFab). The observed temperature drop with the different constructs was further substantiated by the analysis of the cytokine pattern elicited during the FIR. As shown in FIG. 6C only the construct BS-noFab causing a drop in temperature also display elevated cytokine levels. In contrast thereto, the standard BS-mAb construct did not cause cytokine up-regulation when administered to huFcγR mice (FIG. 6C). The cytokine profile for BS-mAb is comparable to that obtained with the effector-dead construct (cf. FIG. 5D). This illustrates the importance of presenting the Fc-region of the IgG in an appropriate position to engage with FcγRs.

Thus, it has been found that both therapeutic target binding Fabs arms are required to maximize the inhibitory effect on FcγR recruitment in order to minimize FIR and cytokine release.

The dose-response was also investigated for the BS-mAb construct (FIG. 6D) and a small and transient effect was detectable at the highest dose (20 mg/kg). This is at a dose which is 10-time higher than the very effective therapeutic dose reducing plaque formation (FIGS. 3E and 3F).

When comparing a standard anti-TfR1 mAb with the BS-noFab (FIG. 6E), the construct lacking both Fab arms induced a stronger temperature drop than the conventional mAb even though the Fc-region is presented in a reverse orientation and the BS-noFab engage in a monovalent state lacking the contribution from avidity binding.

### A Specific Cytokine Signature for the Anti-TfR1 mAb and Diminished Effect by the Brain Shuttle Construct.

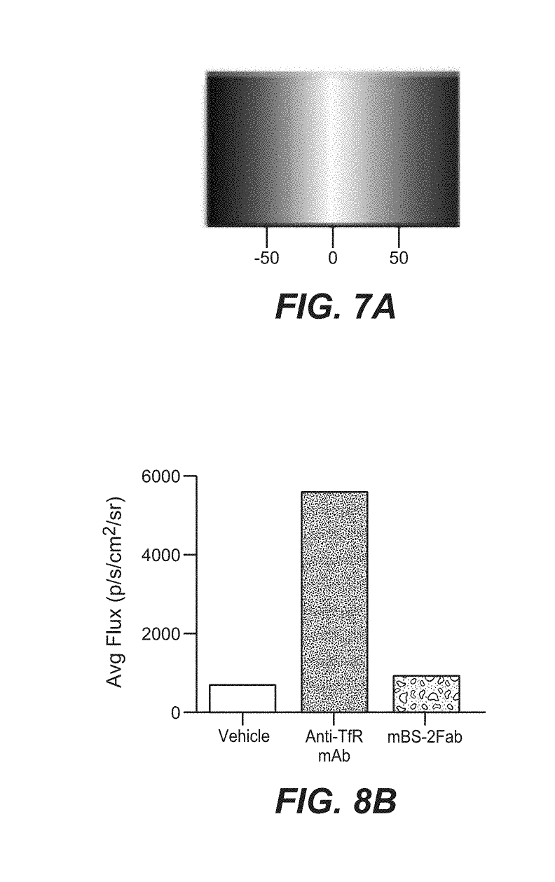

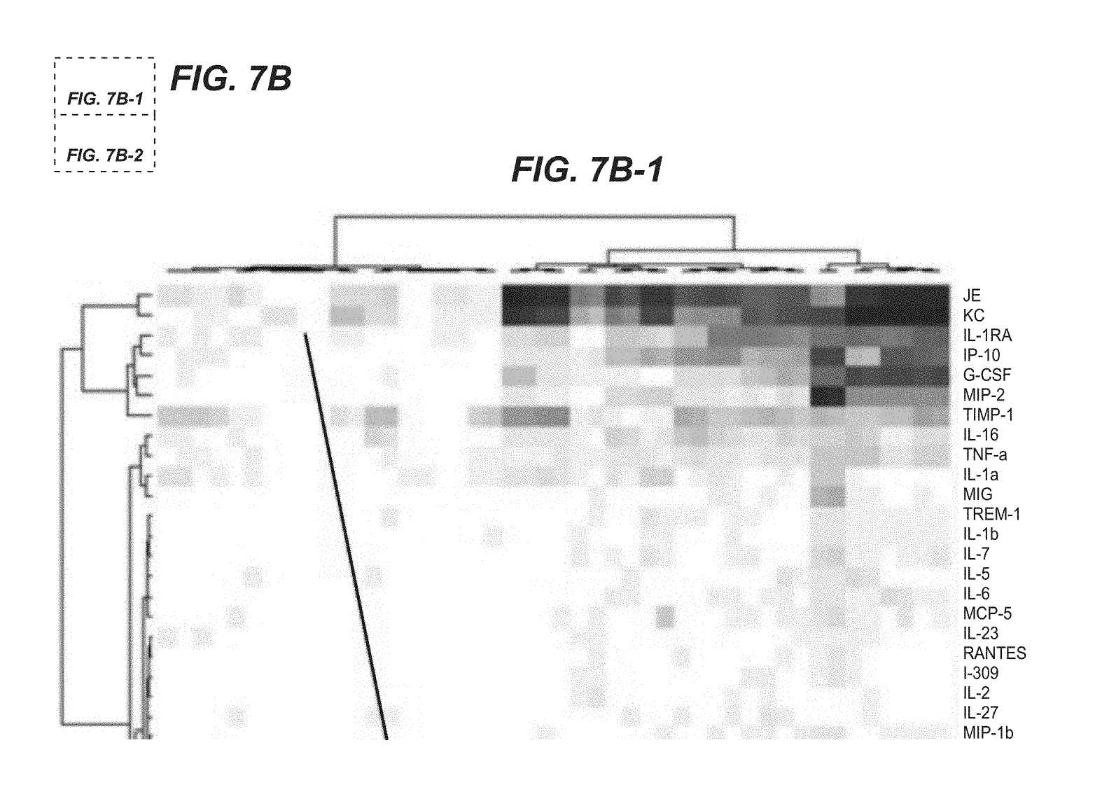

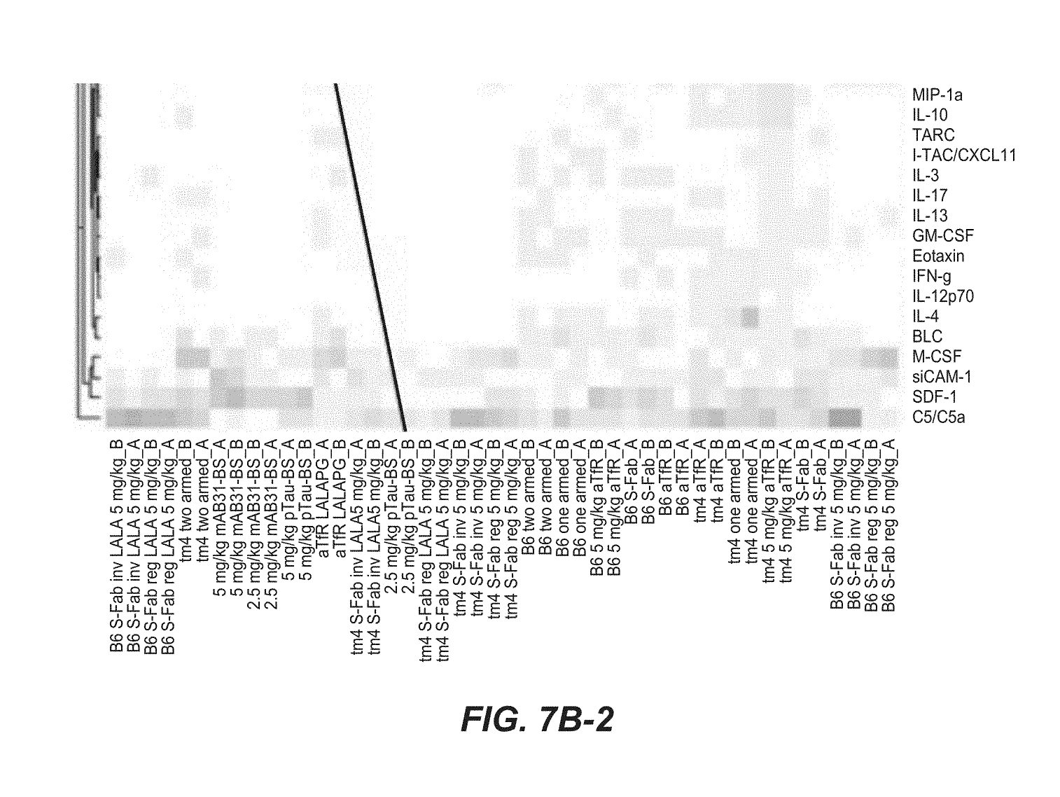

A more detailed analysis of the cytokine profile for the various mAb construct was carry out. A heatmap was generated to highlight key cytokines (FIG. 7A). In particular, two cytokines responded very differently (FIGS. 7B and 7C).

Intravascular Whole Body Optical Imaging Shows that the Brain Shuttle Constructs Attenuate ROS Production.

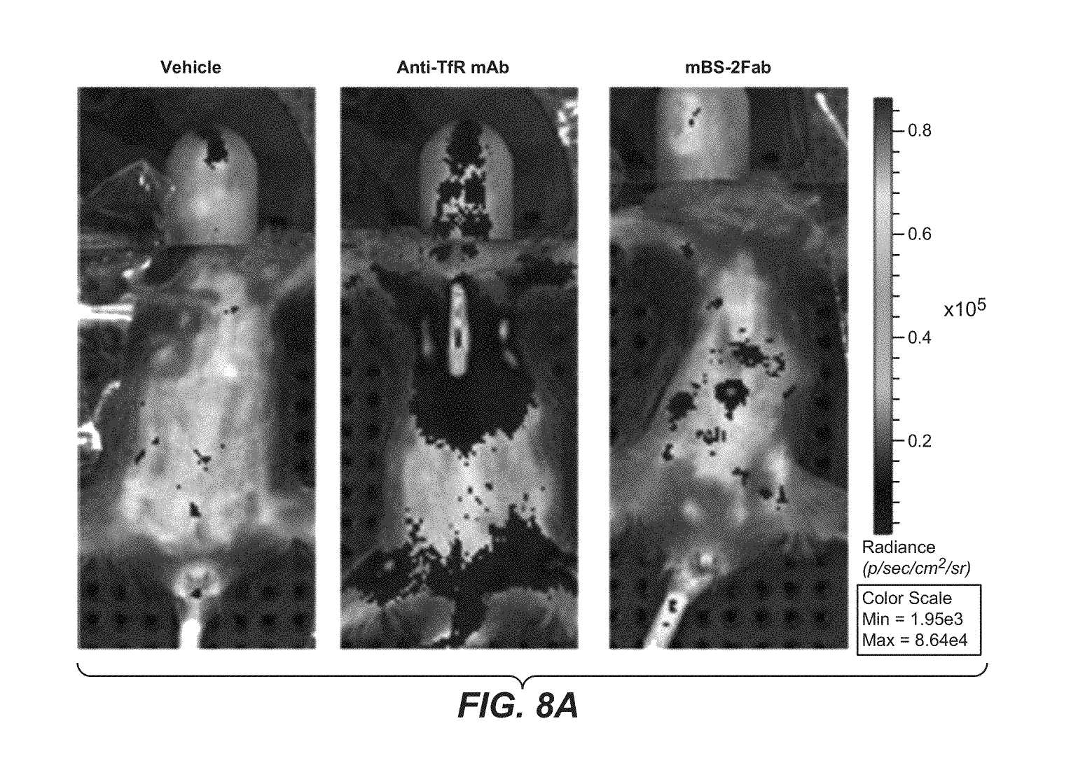

Reactive oxygen species (ROS) are chemically reactive chemical species. After peripheral administration on the standard anti-TfR1 mAb and the Brain Shuttle constructs the whole body was scanned for induction of ROS species. In FIG. 8A, representative images show the difference between the anti-TfR1 mAb and the BS-mAb (mBS-2Fab) construct. The data was quantified and the BS-mAb (mBS-2Fab) showed no significant difference compare to the vehicle group (FIG. 8B).

Structural Modeling of Different mAb Constructs Shows Major Differences in Engagement with FcγR.

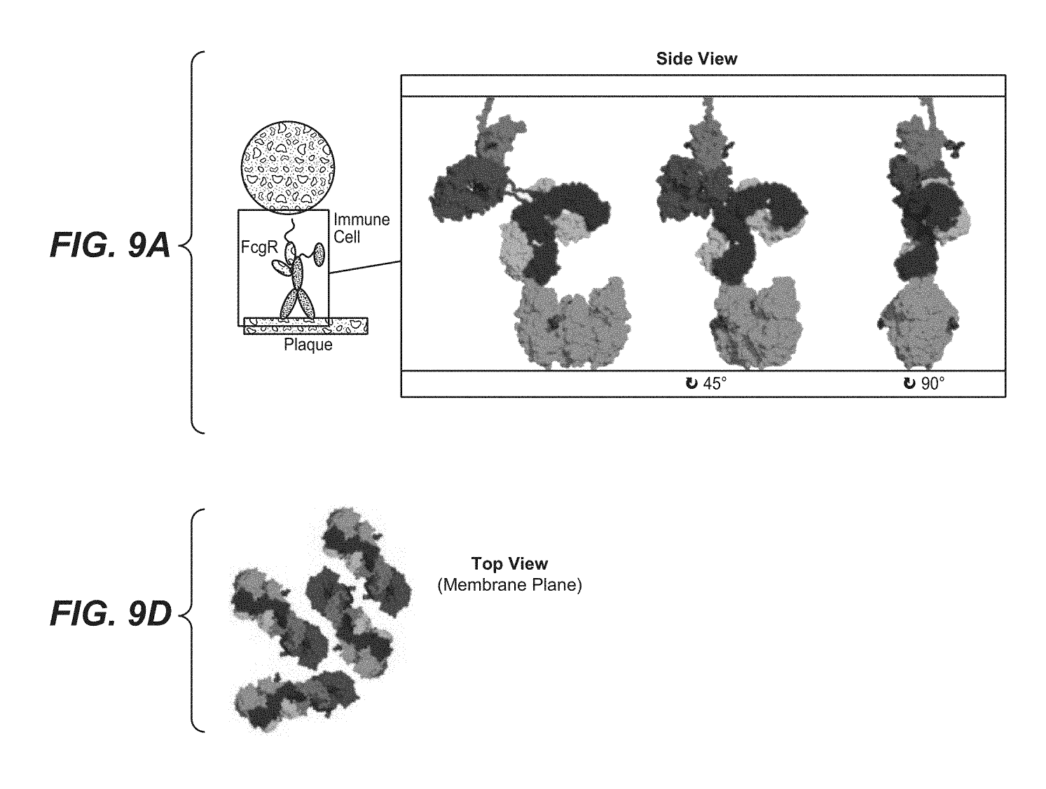

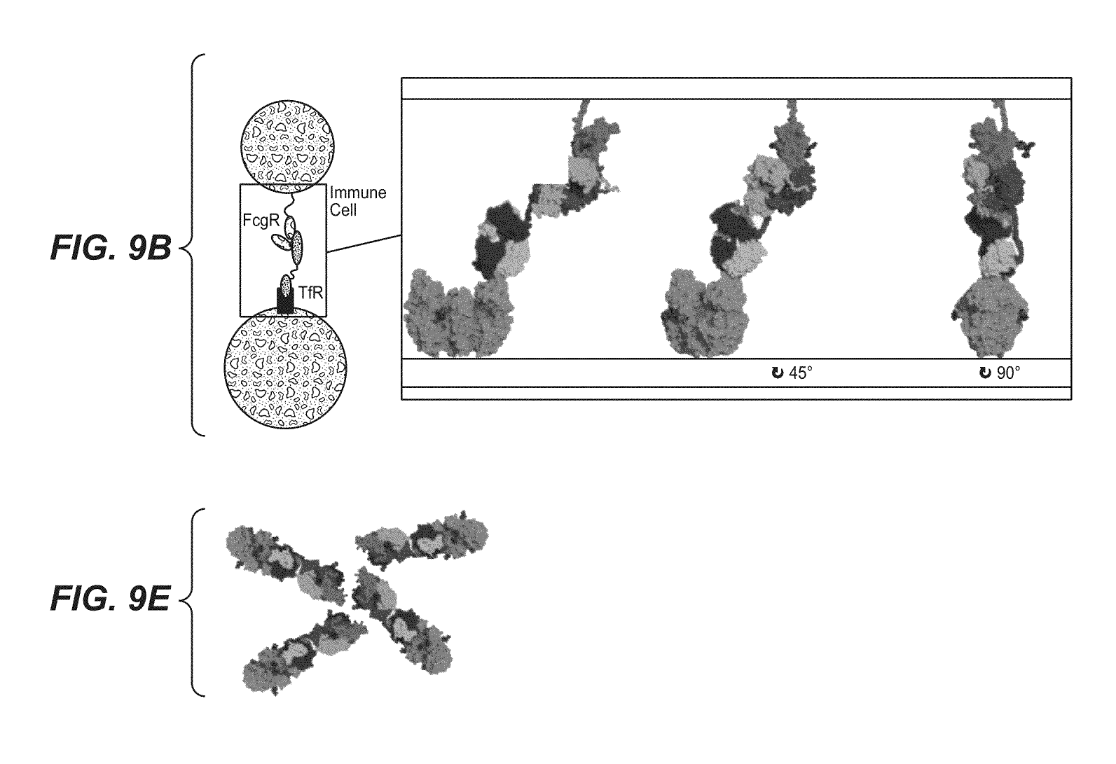

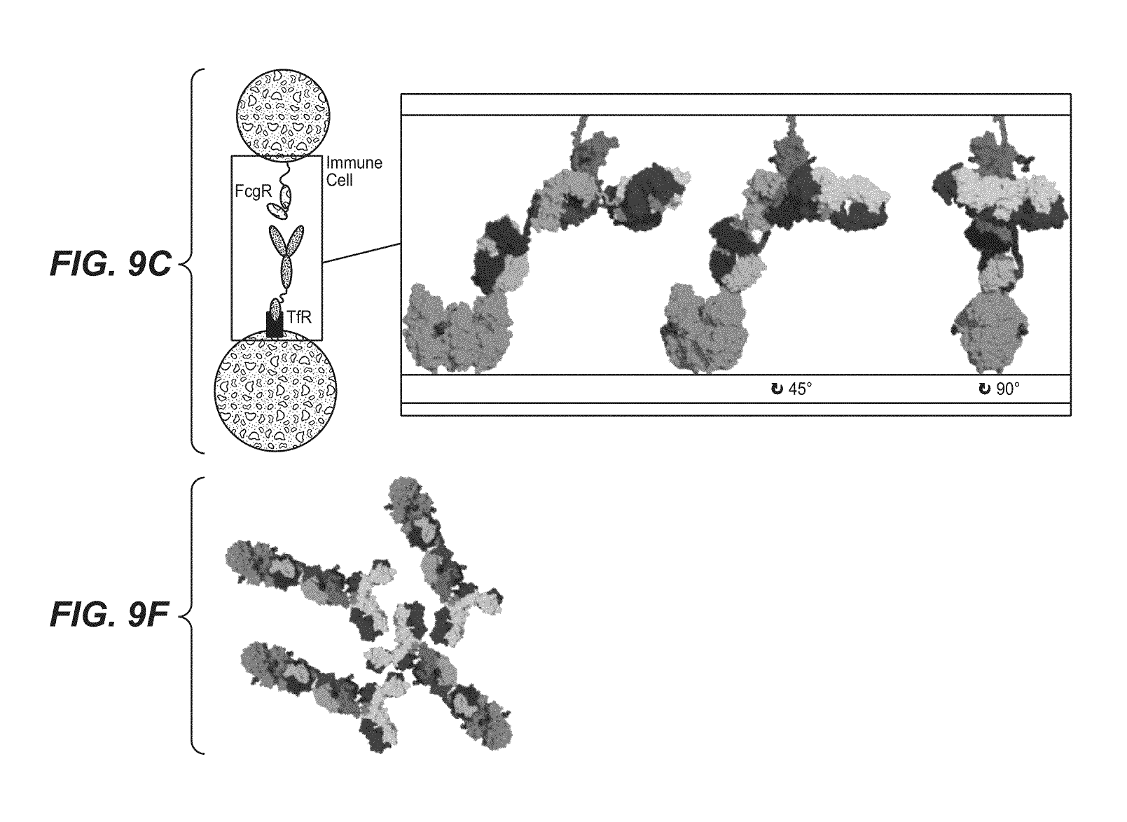

The Fc-region-FcγR interaction between three different construct which is either presented by mAb target binding or BS-module binding on cell surface expressed TfR1 was analyzed using molecular structural information. In FIG. 9 the major observation is summarized.

First, the standard mAb bound to its therapeutic target on one cell surface and the possibility to engage with an FcγR displayed on a neighboring cell surface has been modelled (FIGS. 9A and 9D). The model predicted free access to the FcγR and clustering. Likewise FIGS. 9A and 9D also show that the presence of an additional BS-module (anti-TfR1 CrossFab) at the C-terminus of the standard IgG does not interfere with the FcγR binding.

Second, the BS-noFab construct which is very active in vivo was modelled. This construct bound to TfR1 was presented to the FcγR in a favorable manner and allowed clustering (FIGS. 9B and 9E) in a similar way as the standard mAb when bound to its target.

Third, the standard Brain Shuttle construct (BS-mAb=mBS-2Fab) was modelled. It has been found that the model supports the in vivo findings reported herein that the therapeutic antigen binding Fabs are positioned very close to the FcγR and especially seem to prevent close clustering of the BS-scFab/FcγR complex (FIGS. 9C and 9F).

### Outline

The Fc-region dependent effector functions are in many cases part of the mechanism of action of certain mAbs for therapeutic efficacy in the CNS field. The mAbs bind to their cognate antigens and are in turn recognized by specific Fc-receptors on the cell surface of immune cells. Crosslinking these Fc-receptors leads to activation of several effector cell functions (22). In this way, mAbs are the bridge between the two arms of the immune system, bringing together the specificity of recognition of the adaptive immune system and the destructive potential of the cells of the innate immune system. Examples where effector function could be crucial for the therapeutic effect includes Alzheimer's and Parkinson's disease where aggregated Amyloid-β, phosphorylated tau protein and α-synuclein needs to be removed via FcγR binding and engulfment by microglia. The BS-mAb constructs contain an additional binding domain (BS-module) that will bind TfR1 in peripheral tissues and orientate the mAb in an entirely different arrangement on the surface of cells expressing the transferrin receptor 1 (FIG. 4A).

It has now been found by the current inventors that the BS-mAb is fully capable of stimulating effector function when it is bound to its therapeutic target by the Fv portion of the mAb. Thus, the C-terminal attached BS-module on the heavy-chain does not interfere with Fc-FcγR recruitment and binding. The BS-mAb and the parental mAb are equally potent (FIG. 2 shows this for the exemplary anti-Aβ mAb; both antibodies are equally potent in stimulating glial engulfment of Aβ, which has been shown to be directly dependent on the effector function (23)).

Before the BS-mAb can promote its therapeutic effect in the brain the construct will after administration circulate in the blood stream (systemically). Thereby it will engage with TfR1 expressed on numerous cell types (24), as well as being transported across the BBB. This TfR1 engagement in the systemic circulation could potentially create a local inflammatory response involving the effector function of the Fc-region.

By using a novel huFcγR transgenic mouse model, which expresses key huFcγRs recapitulating the human expression profile, FIR as triggered with human/humanized mAbs was assessed as based on an adaptable telemetric monitoring system allowing continuous temperature data collection. The importance of using this humanized FcγR model for investigating human/humanized mAbs is demonstrated in the much lower FIR response found in wild type animals, reflecting the inherent differences between mice and human FcγR.

It has been found that a conventional bivalent anti-TfR1 mAb with a native IgG1 Fc-region provokes a strong FIR (see FIG. 5C). It has also been found that that the FIR related temperature changes are driven by the Fc-region-FcγR interaction, as effector-dead variants are completely inactive.

It has been found that the BS-mAb construct comprising a fully native human IgG1 Fc-region can fully interact with FcγR receptors depending on the binding mode. The BS-mAb is designed to facilitate entry into the CNS through translocation over the BBB via binding to the TfR1 on the luminal part of CNS vessels. Thus, binding of the endothelial TfR1 precedes binding of the brain resident target. Hence, BS-mAb constructs should ideally not elicit systemic adverse effects like FIR due to peripheral engagement of the widely expressed TfR1. After passage of the BBB the same BS-mAb needs to preserve full effector functions upon binding of the locally expressed target antigen, e.g. for microglia aided clearance of plaques. It has now been found that systemic administration of the BS-mAb construct to huFcγR mice did not induce measurable FIRs using the temperature readout, even though this construct binds mouse TfR1 in the periphery and possesses a fully functional Fc-region (FIG. 6B).

It has been found by the current inventors that steric hindrance is the reason for this differential behavior. It has been found that constructs lacking the native Fab arms regained the ability to provoke FIRs even if the Fc-portion is inversed due to the non-natural orientation when the C-terminal BS binds the TfR1.

It has been found that a construct containing only one Fab arm opposite to the BS module showed intermediate FIR effect (FIG. 6B).

Using a modelling method, it has been found that in case of an anti-TfR1 antibody in standard IgG format, binding of one of its anti-TfR1 Fabs to TfR1 on the target cell displays the Fc-region for interactions with FcγR in its natural configuration.

Without being bound by this theory the second non-bound Fab is constrained by the disulfide bridges in the antibody hinge and therefore likely to follow suit in pointing downwards toward the target cell. Alternatively, the second Fab could bind another TfR1 receptor on the target cell.

The Fc-anti-TfR1 Fab C-terminal fusion enables unhindered FcγR interactions as the C-terminal fusion of the anti-TfR1 Fab via a 4×G₄S flexible linker does not interfere with Fc-region-FcγR interactions that mainly involve the N-terminal part of the Fc-region. This is the case in solution as well as upon cell-cell interactions or as in this case target (i.e. Abeta plaque)-cell interaction. Thus, when interacting with its target the interaction of the BS-mAb Fc-region with Fcgamma receptors on effector cells is not influenced by the C-terminally fused brain shuttle module (i.e. monovalent anti-TfR1 antibody).

Without being bound by this theory, the situation is different for the BS-mAb construct when bound to the TfR1, where the two native N-terminal therapeutic target binding Fab fragments (in the absence of a target likely to be approximately in the same plain as their Fc-region) are forming a steric obstacle. While the Fc-region can still achieve binding to a single FcγR, it is likely that the approach of additional FcγRs necessary for FcγR dimerization or multimerization is hindered, so that the formation of ADCC is inhibited. This notion is outlined in FIG. 9, which illustrate that the lateral approach of multiple FcγR molecules is more likely to be achieved for the standard IgG and the Fc-anti-TfR1 Fab C-terminal fusion complexes than for the targeted IgG-anti-TfR1 Fab C-terminal fusion complex. An alternative or complementary explanation is that the natural Fabs on the cargo IgG increase the gap between the cells within the phagocytic cup due to bulkiness and therefore unable to sterically exclude phosphatases outside the diffusion barrier (25, 26). This would prevent the critical separation of phosphatases and kinases at the submicron-scale within the phagocytic cup which is required for activation of down-stream kinase signaling.

Taken together, it has been found that appending of the BS module at the C-terminal end of conventional mAbs does not interfere with the therapeutic effect of a BS-mAb format as mediated by Fc-region and FcγR interaction. It has also been found that in the BS-mAb the Fc-region-mediated effector functions potentially leading to FIR when the TfR1 is engaged in the periphery by the BS module are silenced. This beneficial property of the BS-mAb format is, without being bound by this theory, ascribed to the steric hindrance exerted by the two natural IgG cargo Fab arms at the N-terminal position opposite to the BS module. This unique feature allows further development of BS-mAb fusions with wild-type Fc which are then capable of exerting their desired FcγR-related pharmacology only at its therapeutic target, without the risk of FIRs.

### Pharmaceutical Formulations

Pharmaceutical formulations for the application of an anti-brain target/human transferrin receptor antibody, wherein the anti-brain target/human transferrin receptor antibody has two binding sites (VH/VL pairs) that specifically bind to the brain target, one binding site (VH/VL pair) that specifically binds to the human transferrin receptor and an effector function competent (native) Fc-region, are prepared by mixing such antibody having the desired degree of purity with one or more optional pharmaceutically acceptable carriers (Remington's Pharmaceutical Sciences, 16th edition, Osol, A. (ed.) (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyl dimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as poly(vinylpyrrolidone); amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rhuPH20 (HYLENEX®, Baxter International, Inc.). Certain exemplary sHASEGPs and methods of use, including rhuPH20, are described in US 2005/0260186 and US 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

The formulations to be used for in vivo administration are generally sterile. Sterility may be readily accomplished, e.g., by filtration through sterile filtration membranes.

### Therapeutic Methods and Compositions

In one aspect, an anti-brain target/human transferrin receptor 1 antibody, wherein the anti-brain target/human transferrin receptor 1 antibody has two binding sites (VH/VL pairs) that specifically bind to the brain target, one binding site (VH/VL pair) that specifically binds to the human transferrin receptor 1 and an effector function competent (native) Fc-region, for use in treating a neurological disorder with reduced/prevented infusion-related drop of the body-temperature is provided. In certain embodiments, an anti-brain target/human transferrin receptor 1 antibody, wherein the anti-brain target/human transferrin receptor 1 antibody has two binding sites (VH/VL pairs) that specifically bind to the brain target, one binding site (VH/VL pair) that specifically binds to the human transferrin receptor 1 and an effector function competent (native) Fc-region, for use in a method of treatment of a neurological disorder with reduced/prevented infusion-related drop of the body-temperature is provided. In certain embodiments, the invention provides an anti-brain target/human transferrin receptor antibody, wherein the anti-brain target/human transferrin receptor 1 antibody has two binding sites (VH/VL pairs) that specifically bind to the brain target, one binding site (VH/VL pair) that specifically binds to the human transferrin receptor 1 and an effector function competent (native) Fc-region, for use in a method of treating an individual having a neurological disorder comprising administering to the individual an effective amount of the anti-brain target/human transferrin receptor 1 antibody, wherein the anti-brain target/human transferrin receptor 1 antibody has two binding sites (VH/VL pairs) that specifically bind to the brain target, one binding site (VH/VL pair) that specifically binds to the human transferrin receptor 1 and an effector function competent (native) Fc-region, wherein the infusion-related drop of the body-temperature is reduced/prevented. In one such embodiment, the method further comprises administering to the individual an effective amount of at least one additional therapeutic agent. In further embodiments, the invention provides an anti-brain target/human transferrin receptor 1 antibody, wherein the anti-brain target/human transferrin receptor 1 antibody has two binding sites (VH/VL pairs) that specifically bind to the brain target, one binding site (VH/VL pair) that specifically binds to the human transferrin receptor 1 and an effector function competent (native) Fc-region, for use in reducing/preventing infusion-related drop of the body-temperature. In certain embodiments, the invention provides an anti-brain target/human transferrin receptor 1 antibody, wherein the anti-brain target/human transferrin receptor 1 antibody has two binding sites (VH/VL pairs) that specifically bind to the brain target, one binding site (VH/VL pair) that specifically binds to the human transferrin receptor 1 and an effector function competent (native) Fc-region, for use in a method of reducing infusion-related drop of the body-temperature in an individual comprising administering to the individual an effective of the anti-brain target/human transferrin receptor 1 antibody, wherein the anti-brain target/human transferrin receptor 1 antibody has two binding sites (VH/VL pairs) that specifically bind to the brain target, one binding site (VH/VL pair) that specifically binds to the human transferrin receptor 1 and an effector function competent (native) Fc-region. An “individual” according to any of the above embodiments is preferably a human.

In a further aspect, the invention provides a method for treating a neurological disorder. In one embodiment, the method comprises administering to an individual having such a neurological disorder an effective amount of an anti-brain target/human transferrin receptor 1 antibody, wherein the anti-brain target/human transferrin receptor 1 antibody has two binding sites (VH/VL pairs) that specifically bind to the brain target, one binding site (VH/VL pair) that specifically binds to the human transferrin receptor 1 and an effector function competent (native) Fc-region. In one such embodiment, the method further comprises administering to the individual an effective amount of at least one additional therapeutic agent. An “individual” according to any of the above embodiments may be a human.

In a further aspect, the invention provides a method for reducing infusion-related body-temperature drop in an individual. In one embodiment, the method comprises administering to the individual an effective amount of an anti-brain target/human transferrin receptor 1 antibody, wherein the anti-brain target/human transferrin receptor 1 antibody has two binding sites (VH/VL pairs) that specifically bind to the brain target, one binding site (VH/VL pair) that specifically binds to the human transferrin receptor 1 and an effector function competent (native) Fc-region. In one embodiment, an “individual” is a human.

The anti-brain target/human transferrin receptor 1 antibody, wherein the anti-brain target/human transferrin receptor 1 antibody has two binding sites (VH/VL pairs) that specifically bind to the brain target, one binding site (VH/VL pair) that specifically binds to the human transferrin receptor 1 and an effector function competent (native) Fc-region, can be used either alone or in combination with other agents in a therapy. For instance, such an antibody may be co-administered with at least one additional therapeutic agent.

The anti-brain target/human transferrin receptor 1 antibody, wherein the anti-brain target/human transferrin receptor 1 antibody has two binding sites (VH/VL pairs) that specifically bind to the brain target, one binding site (VH/VL pair) that specifically binds to the human transferrin receptor 1 and an effector function competent (native) Fc-region, would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an anti-brain target/human transferrin receptor 1 antibody, wherein the anti-brain target/human transferrin receptor 1 antibody has two binding sites (VH/VL pairs) that specifically bind to the brain target, one binding site (VH/VL pair) that specifically binds to the human transferrin receptor 1 and an effector function competent (native) Fc-region, (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.5 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

Typical infusion rates for the administration of the bispecific antibody of the invention are between 50 ml/h and 400 ml/h, in particular ≥50 ml/h, ≥100 ml/h, ≥150 ml/h or ≥200 ml/h; e.g. between 100 ml/h and 400 ml/h, between 150 ml/h and 400 ml/h or between 200 ml/h and 400 ml/h.

The following examples and the figures herein are provided to aid the understanding of the present invention, the true scope of which is set forth in the appended claims. It is understood that modifications can be made in the procedures set forth without departing from the spirit of the invention.

### LIST OF CITED REFERENCES

1. Lajoie, J. M. and Shusta, E. V., Ann. Rev. Pharmacol. Toxicol. 55 (2015) 613-631.
2. Pardridge, W. M., Clin. Pharmacol. Ther. 97 (2015) 347-361.
3. Jefferies, W. A., et al., Nature 312 (1984) 162-163.
4. Friden, P. M., et al., Science 259 (1993) 373-377.
5. Fishman, J. B., et al., J. Neurosci. Res. 18 (1987) 299-304.
6. Pardridge, W. M., et al., J. Pharmacol. Exp. Ther. 259 (1991) 66-70.
7. Roberts, R. L., et al., J. Cell. Sci. 104 (1993) 521-532.
8. Yu, Y. J., et al., Science Translat. Med. 3 (2011) 84ra44.
9. Niewoehner, J., et al., Neuron 81 (2014) 49-60.
10. Bien-Ly, N., et al., J. Exp. Med. 211 (2014) 233-244.
11. Yu, Y. J., et al., Science Translat. Med. 6 (2014) 261ra154.
12. Couch, J. A., et al., Science Transl. Med. 5 (2013) 183ra157, 181-112.
13. Bruhns, P., et al., Blood 113 (2009) 3716-3725.
14. Bohrmann, B., et al., J. Alzheimer's Dis. 28 (2012) 49-69.
15. Bard, F., et al., Nature Med. 6 (2000) 916-919.
16. Nicoll, J. A., et al., Nature Med. 9 (2003) 448-452.
17. Boche, D., et al., Brain 131 (2008) 3299-3310.
18. Dewachter, I., et al., Exp. Geront. 35 (2000) 831-841.
19. Zhang, Y., et al., J. Neurosci. 34 (2014) 11929-11947.
20. Wines, B. D., et al., J. Immunol. 164 (2000) 5313-5318.
21. Friden, P. M., et al., Proc. Natl. Acad. Sci. USA 88 (1991) 4771-4775.
22. Nimmerjahn, F. and Ravetch, J. V., Nat. Rev. Immunol. 8 (2008) 34-47.
23. Adolfsson, O., et al., J. Neurosci. 32 (2012) 9677-9689.
24. Ponka, P., et al., Semin. Hematol. 35 (1998) 35-54.
25. Freeman, S. A., et al., Cell 164 (2016) 128-140.
26. Chang, V. T., et al., Nature Immunol. 17 (2016) 574-582.
27. Saphire, E. O., et al., Science 293 (2001) 1155-1159.
28. Bujotzek, A., et al., mAbs 7 (2015) 838-852.
29. Ramsland, P. A., et al., J. Immunol. 187 (2011) 3208-3217.
30. Lawrence, C. M., et al., Science 286 (1999) 779-782.
31. Rayner, L. E., et al., J. Biol. Chem. 290 (2015) 8420-8438.
32. Saphire, E. A., et al., Science 293 (2001) 1155-1159.

These and all other patent and non-patent references cited herein are herewith incorporated by reference in their entirety.

### EXAMPLES

### Example 1

### Brain Shuttle Constructs

Antibody constructs were generated by cloning cDNAs coding for IgG heavy and light chains, respectively, into mammalian expression vectors. All antibody constant regions were human, variable regions human or rat, depending on the antibodies used. Fab fusions to the Fc C-terminus were achieved by fusing a single-chain Fab construct, where heavy and light chains were connected by a G4S linker, to the 3′ terminus of the IgG heavy chain, again via G4S linker. Asymmetric constructs were obtained using knob-into-hole technology (Ridgway et al., 1996). Constructs were expressed in HEK293 or CHO-K1 cells and purified by standard Protein A affinity followed by size-exclusion chromatography (SEC). Antibody preparations were routinely analyzed by capillary electrophoresis and SEC, and endotoxin content measured.

The following constructs have been produced accordingly and used in the herein reported examples:

### Example 2

### Binding Assessment of Fcγ Receptors by Surface Plasmon Resonance

For FcγR measurement a SPR capture assay was used. Around 5000 resonance units (RU) of the capturing system (10 μg/ml Penta-His; Quiagen cat. No. 34660) were coupled on a CM5 chip (GE Healthcare BR-1005-30) at pH 5.0 by using an amine coupling kit supplied by the GE Healthcare. The sample and system buffer was PBS-T+pH 7.4. The flow cell was set to 25° C.—and sample block to 12° C.—and primed with running buffer twice. The FcγR-His-receptor was captured by injecting a 5 μg/ml solution for 60 sec. at a flow of 10 μl/min. Binding was measured by injection of 100 nM of antibody sample for 180 sec at a flow of 10 μl/min. The surface was regenerated by 30 sec washing with 10 mM Glycine pH 1.7 solution at a flow rate of 10 μl/min. With this assay binding of either IgG or BS-IgG construct to FcγR was determined.

### Example 3

### Isolation of Primary Human Cells and Phagocytosis Assay

Monocytes were obtained from human peripheral blood mononuclear cells (PBMCs) from a buffy coat (obtained from a local blood bank) by Ficoll density centrifugation. Monocytes were isolated from PBMCs by magnetic labeling using MACS® separation (Miltenyi Biotec, Germany #130-091-153) that consists of the Monocyte Isolation Kit II for isolation of human monocytes through depletion of non-monocytes (negative selection). Monocytes were differentiated to macrophages by adding 0.3 g/mL human macrophage colony stimulating factor (GenScript Z02001). Differentiated human macrophages were cultured in RPMI 1640 (Gibco #61870-044) medium with 100 U/mL penicillin and 100 μg/mL streptomycin (Gibco #15140-122). Differentiated macrophages were incubated in an antibody-dependent cellular phagocytosis assay employing cryosectioned postmortem human AD brain sections as substrate. Human AD brain tissue sections from cortical regions (Braak stage VI) were prepared at a nominal thickness of 20 μm and placed onto removable poly-D-lysine coated 2-well culture dishes (Biocoat™ #40629). Brain sections were pre-incubated with different concentrations of Gantenerumab for 1 h, washed with PBS before human primary cells were seeded at 0.8 to 1.5×10⁶ cells/mL and cultured at 37° C. with 5% carbon dioxide for 2 to 3 days. An unrelated human IgG1 (Serotec, PHP010) antibody was used as an additional control. Detection of amyloid plaques was done after fixation with 2% formaldehyde for 10 min, washing and staining with BAP2 conjugated to AlexaFluor488 at 10 g/ml for 1 h at room temperature. Double-labeling of macrophages was done with antibodies against A and Gantenerumab as described above and lysosomal marker antibody against LAMP2 (RDI Division of Fitzgerald Industries Intl).

### Example 4

### Immunohistochemistry

Brains were prepared after PBS perfusion and sagittal cryo-sections were cut between lateral 1.92 and 1.68 millimeter according to the brain atlas of Paxinos and Franklin. Brains were sectioned at a nominal thickness of 20 microns at −15° C. using a Leica CM3050 S cryostat and placed onto precooled glass slides (Superfrost plus, Menzel, Germany). For each brain, three sections spaced 80 microns were deposited on the same slide.

Sections were rehydrated in PBS for 5 minutes followed by immersion with 100% acetone precooled to −20° C. for 2 min. All further steps were done at room temperature. Slides with brain sections were washed with PBS, pH 7.4 and blocking of unspecific binding sites by sequential incubation in Ultra V block (Lab Vision) for 5 minutes followed by PBS wash and incubation in power block solution (BioGenex) with 2% normal goat serum in PBS for 20 min. Slides were directly incubated with the secondary antibody, an affinity-purified goat anti-human IgG (heavy and light chain specific) conjugated to Alexa Fluor 555 dye (# A-21433, lot 54699A, Molecular Probes) at a concentration of 20 μg/ml in 2% normal goat serum in PBS, pH 7.4 for 1 hour. After extensive washing with PBS, plaque localization was assessed by a double-labeling for Abeta plaques by incubation with BAP-2, a Roche in-house murine monoclonal antibody against Abeta conjugated to Alexa Fluor 488 dye at 0.5 μg/ml for 1 hour in PBS with power block solution (BioGenex) and 10% normal sheep serum. After PBS washing, autofluorescence of lipofuscin was reduced by quenching through incubation in 4 mM CuSO4 in 50 mM ammonium acetate, pH 5 for 30 minutes. After rinsing the slides with double-distilled water, slides were embedded with Confocal Matrix (Micro Tech Lab, Austria).

### Example 5

### Microscopy and Image Processing

Three images from each section of the brain of each PS2APP-mouse with plaque containing regions in the frontal cortex (region of the primary motor cortex) were taken. Images were recorded with a Leica TCS SP5 confocal system with a pinhole setting of 1 Airy. Plaques immunolabelled with Alexa Fluor 488 dyes were captured in the same spectral conditions (a 488 nm excitation and a 500-554 nm band pass emission) with adjusted photomultiplier gain and offset (typically, 770 V and −0% respectively) at a 30% laser power. Bound secondary Alexa Fluor 555 antibodies on the accessible surface of tissue sections were recorded at the 561 nm excitation laser line at a window ranging from 570 to 725 nm covering the emission wavelength range of the applied detection antibody. Instrument settings were kept constant for image acquisitions to allow comparative intensity measurements for tested human anti-Aβ antibodies; in particular, laser power, scanning speed, gain and offset. Laser power was set to 30% and settings for PMT gain were typically 850 V and a nominal offset of 0%. This enabled visualization of both faint and strongly stained plaques with the same setting. Acquisition frequency was at 400 Hz. Confocal scans were recorded as single optical layers with a HCX PL APO 20× 0.7 IMM UV objective in water, at a 512×512 pixel resolution and an optical measuring depth in the vertical axis was interactively controlled to ensure imaging within the tissue section. Amyloid-β plaques located in layers 2-5 of the frontal cortex were imaged and fluorescent intensities quantified.

### Example 6

### Statistical Analysis

Immunopositive regions were visualized as TIFF images and processed for quantification of fluorescence intensity and area (measured in pixels) with ImageJ version 1.45 (NIH). For quantification, background intensities of 5 were subtracted in every image and positive regions smaller than 5 square pixels were filtered out. Total fluorescence intensity of selected isosurfaces was determined as sum of intensities of single individual positive regions and the mean pixel intensity was calculated dividing the total intensity by the number of pixels analyzed. Average and standard deviations values were calculated with Microsoft Excel (Redmond/WA, USA) from all measured isosurfaces obtained from nine pictures taken from three different sections for each animal. Statistical analysis was performed using the Student's t test for group comparison or a Mann-Whitney test.

### Example 7

### Pharmacokinetic Studies

C57BL6 male mice of average 30 g weight were used to conduct pharmacokinetic investigations of both mAb31 and BS-mAb31. The respective anybody was administered and an intravenous bolus to mice at 5 and 10 mg/kg respectively (n=3 mice per drug). K2 EDTA plasma samples were prepared at various time points using capillary microsampling to allow full plasma pharmacokinetic profiles across 2 weeks for each mouse. Samples were analyzed using an anti-human CH1/CL1 (kappa) capture/detection immunoassay to determine quantities of drug. Concentration-time profiles were analyzed using Pharsight Phoenix 64, using a two compartment pharmacokinetic model. Chronic dosing profiles were then simulated using pharmacokinetic parameters determined from the single dose PK data at the appropriate doses used.

### Example 8

### ADCC Assay

Transferrin receptor 1 expressing (TfR1+) BaF3 cells (DSMZ, # CLPZ04004) were used as target cells for antibody-dependent cell toxicity (ADCC) experiments induced by different antibodies and antibody-fusion molecules. Briefly, 1×10⁴BaF3 cells were seeded in round bottom 96-wells and optionally co-cultured with human NK92 effector cells (high affinity CD16 clone 7A2F3; Roche GlycArt) at an effector/target ratio of 3:1 in the presence or absence of indicated antibodies. After four hours' incubation (at 37° C., 5% CO₂), cytotoxicity was assessed as measured by the release of lactate dehydrogenase (LDH) from dead/dying cells. For this cells were centrifuged for 5 min at 250×g and 50 μl supernatant was transferred to a flat bottom plate. 50 μl LDH reaction mix (Roche LDH reaction mix, cat. no. 11644793001; Roche Diagnostics GmbH) was added and the reaction was incubated for 20 min at 37° C., 5% CO₂. Subsequently, the absorbance was measured at a Tecan Sunrise Reader at 492/620 nm wavelength.

All samples were tested in triplicates and the specific Killing/ADCC was based the following calculations and controls:
Only target cells (+ medium)
Maximal LDH release: target cells+3% Triton-X
Spontaneous release: target cells+NK cells (E:T of 3:1)
% specific ADCC/lysis was calculated by the following term:

$$\% spec . ADCC = \frac{Sample - spontaneous release}{Maximal release - spontaneous release} \times 100$$

### Example 9

### Monocyte Activation Assay

96-well cell culture plates were coated with Aβ1-42 peptide (Bachem; 20 μg/mL in PBS) over night, then incubated with anti-Aβ antibody solutions for 1 h at 37° C. After washing the plates, 105 U-937 human monocytes, that had been pre-activated with 400 U/mL interferon-γ overnight to upregulate Fcγ receptors, were added per well and plates were incubated for 24 h at 37° C./5% CO₂. The next day, supernatants were transferred to ELISA plates for determination of IL-8 and IP-10 concentrations according to the manufacturer's protocols (R&D Systems).

### Example 10

### Confirmation of FcγRx Binding by FACS Analysis

To confirm whether an IgG and BS-IgG were able to bind to cellular expressed FcγR subtypes, we used in-house generated recombinant CHO cell clones stably expressing FcγRI (CHO-K1_flhFcγRI), FcγRIIa (CHO-K1 flhFcγRIIa_LR), FcγRIIB (CHO-K1_flhFcγRIIb) or FcγRIIIa (CHO-K1_flhFcγRIIIa). CHO cells were grown according to standard cell culture conditions in supplemented EMDM (PAN Biotech). 1×105 CHO cells/well were seeded into a 96-well round bottom plate and incubated with different concentrations of indicated antibody variants in medium for 45 min on ice. A human IgG1 (Sigma, #I5154) was used as isotype control. After washing, cells were re-suspended in 200 μl medium and incubated with 10 μg/ml of AlexaFluor488-conjugated goat anti-human IgG-F(ab′)2 fragment (Jackson, #109-546-006) for additional 45 min. on ice. Then cells were washed twice with medium, re-suspended in 200 μl medium and analyzed for binding to respective FcγRII on a FACS-Canto-II (BD).

### Example 11

### Temperature Study in FcγR-Humanized Mice

The FcγR humanized mice were employed to determine infusion-related side effects. For the in vivo temperature measurement a telemetric temperature measurement system was used: We used the BMDS IPTT300 temperature telemetry system in combination with the DAS-7006 reader system. This chip based telemetry system was implanted to the mice approximately two weeks prior to the experiment. Prior to the experiment the baseline temperature of all individuals was measured. After the i.v. test compound injection, the body temperature was measured in intervals of 5 minutes.

### Example 12

### Cytokine Assay and Analysis

The serum cytokine levels were assesses using the R&D cytokine array panel A, which provides a 40-plex analysis of inflammatory markers. 200 Microliters of pooled serum was used per group. The assay was performed according to the manufacturer's protocol. For the analysis: All relative intensities measured are expressed as percentage of the membrane internal positive control spots. Generally, the displayed values were generated by subtracting the buffer control from the condition of interest.

### Example 13

### Whole Body ROS Imaging

For whole body ROS imaging the PerkinElmer IVIS Spectrum CT was used. ROS detection was done via the PerkinElmer inflammation probe. In brief, the mice were injected 10 min prior to their intended time-point of measurement intraperitoneally with the PerkinElmer inflammation probe. Directly before acquisition the mice were injected with the test construct and imaged under isoflurane anesthesia. The image acquisition was done with an exposure time of 5 min, F1 aperture and medium binning.

### Example 14

### Molecular Modeling

The IgG and IgG-derived structures were created based on the full IgG crystal structure with PDB ID 1HZH (27). The structure of the variable regions was modeled with the antibody homology modeling protocol MoFvAb (28). The Fc-region-FcγR binding mode was adopted from the crystal structure of the human Fc-region of IgG1 in complex with FcγRIIa (PDB ID 3RY6 (29)). The homology model of the mTfR1 homodimer was modeled from the 3.2 Å crystal structure of the hTfR1 extra-cellular domain with PDB ID 1CX8 (30) (77% sequence identity, 88% sequence similarity). The binding mode of the anti-mTfR1 brain-shuttle Fab to mTfR1 was approximated based on an epitope sequence identified by peptide mapping experiments.

Antibody hinge conformations (Ca atoms only) were adopted from a set of IgG solution NMR states published recently (31) and chosen such as to minimize steric clashes with the remainder of the model. All molecular models were generated and visualized using BIOVIA Discovery Studio 4.5 by Dassault Systémes, and arranged and post-processed with GIMP, the GNU Image Manipulation Program.

### Example 15

### Binding Studies

First, a surface plasmon resonance (SPR) based assay was used wherein four different FcγR were immobilized in the flow channels as the immobilized target. The SPR results showed that both constructs bind the different FcγRs similar and according to low and high affinity receptors (see FIGS. 1B and 1C).

Second, cell binding experiments were performed wherein the different human FcγR was overexpressed on the cell. Both the BS-mAb31 and the parental mAb31 bound equally well (FIGS. 1D and 1E) and the rank-order was in agreement with the SPR data.

An antibody-dependent cellular cytotoxicity (ADCC) assay with AP was coated on a surface and a monocytic cell was used as an effector cell presenting FcγRs. Two different cytokines were used as readout for cytotoxicity. It was found that BS-mAb31 had a potency comparable to parental mAb31 (see FIGS. 2B and 2C).

In a phagocytosis assay postmortem Alzheimer's disease (AD) brain tissue slices cultured with primary human effector cells were employed (14). AD brain sections were pre-incubated with different concentrations of BS-mAb31 and parental mAb31 followed by incubation with effector cells. A concentration-dependent decrease of Aβ plaque load was observed (see FIGS. 2D and 2K) for both antibodies.

### Example 16

In Vivo Efficacy in Brain of mAb31

Plaque reduction properties of the BS-mAb31 construct versus the parental mAb31 were investigated in a transgenic amyloidosis mouse model (APP London: APP V717I) (18). The plasma exposure was lower for the BS-mAb31 compared to mAb31 (see FIG. 3A).

A 4-month efficacy study was designed based on weekly dosing and the plasma exposure was simulated (see FIG. 3B). Target engagement in the cortex of the anti-Aβ mAb and its Brain Shuttle construct after 4-months of dosing every week was investigated. It was substantially much more plaque decoration detectable with the BS-mAb31 (FIGS. 2C and 2D). In this 4-month chronic treatment study a significant reduction of Aβ amyloid plaques in cortex and hippocampus was visible in BS-mAb31 treated mice compared with vehicle controls and equimolar low-dose of mAb31 even though plasma exposure for the Brain Shuttle was substantial lower (see FIGS. 2E and 2F). This data shows that the attached BS-module at the C-terminus of the mAb31 does not interfere with the interaction with the FcγR on microglia cells. Thereby Aβ plaque clearance is promoted besides significantly improved in vivo efficacy by enhanced brain exposure of the therapeutic IgG (9).

### Example 17

TfR1 Binding Mode Attenuates the Engagement with FcγRs.

The in vitro TfR1 binding properties of the BS-mAb31 and the anti-TfR1 mAb were investigated. The BS-mAb31 construct contains an anti-TfR1 Fab as the C-terminal BS module. It has been found that the binding to TfR1 of the BS-mAb31 (FIG. 4A) and the bivalent native anti-TfR1 mAb (FIG. 4B) is different resulting in a different spatial presentation of the therapeutic entity (IgG) and the Fc-region towards the environment. The functionality of the Fc-region when the construct is bound to the TfR1 was determined using an antibody-dependent cell-mediated cytotoxicity (ADCC) assay. In this assay one cell expresses the TfR1 and the other cell (human NK92) has the function of an effector cell expressing FcγRIIIA ADCC is a mechanism of cell-mediated immune defense whereby an effector cell of the immune system actively lyses a target cell, whose membrane-surface antigens have been bound by specific antibodies.

The interaction has been analyzed using three different IgG constructs. The standard anti-TfR1 mAb with full effector function produced a strong ADCC response. The anti-TfR1 one Fab mAb also produced an ADCC response but at a higher concentration due to loss of avid binding (FIG. 4C). All cytotoxicity effect was mediated by the Fc-region. Confirmation was done using an anti-TfR1 mAb with no effector function (P329G/L234A/L235A mutation in the Fc-region). This antibody had no effect in this ADCC assay. Interestingly, the two Brain Shuttle constructs with one or two BS modules fused to the C-terminus of the heavy chains of mAb31, had none or very low level of cytotoxicity (FIG. 4C). At the concentration of the standard anti-TfR1 mAb, which provoked the highest ADCC effect, only a small effect was detected for the anti-TfR1 one Fab mAb, whereas all other constructs did not have a detectable effect (FIG. 4D).

### Example 18

### First Infusion Reactions and Cytokine Inductions

It was determined what consequences effector function will have when the BS-antibody binds to the TfR1 through the BS-module.

In the first step this was examined in a huFcγR transgenic mouse system. In short, this model was generated through gene-targeted replacement of the two activating low-affinity mouse FcγR genes (Fcγr3 and Fcγr4) by the four human counterparts (FCGR2A, FCGR3A, FCGR2C and FCGR3B) (FIG. 5A). This provides an adequate system to evaluate in vivo the potential interaction between human/humanized mAbs and human FcγRs resulting in the triggering of effector functions. The model uses telemetric temperature readout (FIG. 5B) to monitoring first infusion reactions (FIR). As outlined already above the FIR is induced by the effect of FcγR interactions and recruitment of effector immune cells. The wireless recording system in this model allows the animals to move freely during the study.

First, the FIR as induced by the injection of a conventional anti-TfR1 mAb was determined. As shown if FIG. 5C the injection of the conventional anti-TfR1 mAb resulted in a concentration-dependent and transient decrease in body temperature which returned to normal levels within approximately two hours.

Second, the FIR as induced by the injection of a monovalent form of a conventional anti-TfR1 mAb was determined. The monovalent form of a conventional anti-TfR1 mAb contains only one Fab arm against TfR1. Also this mAb strongly induced FIR.

Third, the relative contribution of effector function to the FIR observed in this model with anti-TfR1 mAb was determined using mAbs with mutations in the Fc-region at residues that are required for FcγR binding (20). The Fc-region triple mutant P329G/L234A/L235A, which lacks FcγR interaction, showed no drop in temperature in the model (FIG. 5D). This is corroborated by the in vitro data using the Fc-region effector function eliminated construct (FIG. 4C).

The levels of different cytokine were determined as a response of administration of the anti-TfR1 mAb. It was found that certain cell signaling molecules strongly increased in concentration (FIG. 5E). In particular, Granulocyte-colony stimulating factor (G-CSF), keratinocyte-derived cytokine (KC), Macrophage Inflammatory Protein (MIP-2) and Interferon gamma-induced protein 10 (IP-10) showed a strong response. These cytokine responses can be correlated amongst other things to neutrophil activation. As seen in the temperature readout experiments, virtually no response on cytokine induction was produced when using the IgG construct with eliminated Fc-region effector function (FIG. 5E).

### Example 19

### Brain Shuttle Binding-Mode Effects

To assess the induction of FIR three different BS-mAb constructs were administered to huFcγR transgenic mice (FIG. 6A). These all had human native IgG1 effector function but differed in the numbers of therapeutically effective Fabs (=binding sites).

Unexpectedly it has been found that no FIR was observed for the standard BS-mAb construct showing that mBS-2Fab does not trigger FcγR activation in the periphery in vivo (see FIG. 6B).

Two constructs with one or both therapeutic target binding Fab arm(s) missing on the mAb portion were designed (see FIG. 6A). These constructs, when applied to the huFcγR transgenic mice, clearly caused FIR, as scored by the rapid and strong temperature drop (FIG. 6B). The temperature drop was even more pronounced for the construct lacking both Fab arms (BS-noFab). The observed temperature drop with the different constructs was further substantiated by the analysis of the cytokine pattern elicited during the FIR. As shown in FIG. 6C only the construct BS-noFab causing a drop in temperature also display elevated cytokine levels. In contrast thereto, the standard BS-mAb construct did not cause cytokine up-regulation when administered to huFcγR mice (see FIG. 6C). The cytokine profile for BS-mAb is comparable to that obtained with the effector-dead construct (see FIG. 5D).

The dose-response was also investigated for the BS-mAb construct (FIG. 6D) and a small and transient effect was detectable at the highest dose (20 mg/kg). This is at a dose which is 10-time higher that the very effective therapeutic dose reducing plaque formation (FIGS. 3E and 3F).

### Example 20

### Specific Cytokine Signature

A more detailed analysis of the cytokine profile for the various mAb construct was carry out. A heatmap was generated to highlight key cytokines (FIG. 7A). In particular, two cytokines responded very differently (FIGS. 7B and 7C).

Intravascular Whole Body Optical Imaging Shows that the Brain Shuttle Constructs Attenuate ROS Production.

Reactive oxygen species (ROS) are chemically reactive chemical species. After peripheral administration on the standard anti-TfR1 mAb and the Brain Shuttle construct the whole body was scanned for induction of ROS species. In FIG. 8A, representative images show the difference between the anti-TfR1 mAb and the mBS-2Fab construct. The data was quantified and the mBS-2Fab showed no significant difference compare to the vehicle group (FIG. 8B).

### Example 21

### Structural Modeling of Different mAb Constructs

The Fc-region-FcγR interaction between three different construct which is either presented by mAb target binding or BS-module binding on cell surface expressed TfR1 was analyzed using molecular structural information. In FIG. 9 the major observation is summarized.

First, the standard mAb bound to its therapeutic target on one cell surface and the possibility to engage with an FcγR displayed on a neighboring cell surface has been modelled (FIGS. 9A and 9D). The model predicted free access to the FcγR and clustering. Likewise FIGS. 9A and 9D also show that the presence of an additional BS-module (anti-TfR1 CrossFab) at the C-terminus of the standard IgG does not interfere with the FcγR binding.

Second, the BS-noFab construct which is very active in vivo was modelled. This construct bound to TfR1 was presented to the FcγR in a favorable manner and allow clustering (FIGS. 9B and 9E) in a similar way as the standard mAb when bound to its target.

Third, the standard Brain Shuttle construct (mBS-2Fab) was modelled. It has been found that the model supports the in vivo findings reported herein that the therapeutic antigen binding Fabs are positioned very close to the FcγR and especially seem to prevent close clustering of the BS-scFab/FcγR complex (FIGS. 9C and 9F).

1-17. (canceled)

18. A method for treating a neurological disorder in a patient comprising administering a bispecific antibody to said patient, wherein the antibody comprises
i) an Fc-region,
ii) two binding sites specifically binding to a first (cell surface) target, and
iii) one binding site specifically binding to a second (cell surface) target,
wherein the treatment has reduced side effect after administration, wherein the administration is an intravenous, subcutaneous, or intramuscular administration, and wherein the side effect is an administration-related side effect selected from the group consisting of vasodilation, bronchoconstriction, laryngeal edema, drop of cardiac pressure, and hypothermia.

19. The method according to claim 18, wherein the treatment has a reduced side effect after administration as compared to a treatment with the same antibody lacking one or two of said binding sites specifically binding to the first (cell surface) target.

20. The method according to claim 18, wherein the administration is by infusion.

21. The method according to claim 20, wherein the infusion rate is ≥50 ml/h.

22. The method according to claim 20, wherein the infusion rate is ≥100 ml/h.

23. The method according to claim 20, wherein the infusion rate is ≥150 ml/h.

24. The method according to claim 18, wherein the binding sites to the first target are both at an N-terminal end of an antibody heavy chain and the binding site to the second target is at the C-terminal end of one of the antibody heavy chains.

25. The method according to claim 18, wherein the antibody comprises
i) a pair of a first antibody light chain and a first antibody heavy chain,
ii) a pair of a second antibody light chain and a second antibody heavy chain, and
iii) an additional antibody fragment selected from the group consisting of scFv, Fab, scFab, dAb fragment, DutaFab and CrossFab,
wherein the pairs of antibody chains of i) and ii) specifically bind to the first target and the additional antibody fragment of iii) specifically binds to the second target.

26. The method according claim 25, wherein the additional antibody fragment of iii) is conjugated either directly or via a peptidic linker to the C-terminus of the antibody heavy chain of i) or ii).

27. The method according to claim 18, wherein the neurological disorder is selected from the group consisting of neuropathy, amyloidosis, cancer, an ocular disease or disorder, viral or microbial infection, inflammation, ischemia, neurodegenerative disease, seizure, behavioral disorders, lysosomal storage disease, Lewy body disease, post poliomyelitis syndrome, Shy-Draeger syndrome, olivopontocerebellar atrophy, Parkinson's disease, multiple system atrophy, striatonigral degeneration, tauopathies, Alzheimer disease, supranuclear palsy, prion disease, bovine spongiform encephalopathy, scrapie, Creutzfeldt-Jakob syndrome, kuru, Gerstmann-Straussler-Scheinker disease, chronic wasting disease, and fatal familial insomnia, bulbar palsy, motor neuron disease, nervous system heterodegenerative disorder, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, Unverricht-Lundborg syndrome, dementia, Pick's disease, spinocerebellar ataxia, cancer of the CNS and/or brain, including brain metastases resulting from cancer elsewhere in the body.

28. The method according to claim 18, wherein the first target is selected from the group consisting of human CD20, human tau protein, phosphorylated human tau protein, human glucocerebrosidase, human alpha-synuclein, and human amyloid beta protein, and the second target is human transferrin receptor 1.

29. The method according to claim 18, wherein the first target is selected from the group consisting of human tau protein, phosphorylated human tau protein, human glucocerebrosidase, human alpha-synuclein, and human amyloid beta protein, and wherein the neurological disorder is selected from the group consisting of Alzheimer's disease, Parkinson's disease, and tauopathies.

30. The method according to claim 18, wherein the binding sites are antibody heavy chain variable domain and antibody light chain variable domain pairs.

31. The method according to claim 18, wherein the antibody comprises an effector function competent Fc-region.

32. The method according to claim 18, wherein ADCC elicited by the bispecific antibody upon administration to a patient is lower than that elicited by a bivalent bispecific antibody that has only one binding site that specifically bind to the first target and one binding site that specifically binds to the second target.

33. The method according to any one of claim 32, wherein the ADCC is 10-fold or more lower.

34. The method according to claim 18, wherein the side effect is hypothermia.

35. The method according to claim 34, wherein the hypothermia is reduced to a drop of body-temperature of less than 0.5° C. at a therapeutic dose.

36. The method according to claim 35, wherein the drop of the body temperature is within 60 minutes after administration.

37. The method according to claim 25, wherein
a) the antibody heavy chains are full length antibody heavy chains of the human subclass IgG1,
b) the antibody heavy chains are full length antibody heavy chains of the human subclass IgG4,
c) one of the antibody heavy chains is a full length antibody heavy chain of the human subclass IgG1 with the mutations T366W and optionally S354C and the other antibody heavy chain is a full length antibody heavy chain of the human subclass IgG1 with the mutations T366S, L368A, Y407V and optionally Y349C,
d) both antibody heavy chains are full length antibody heavy chains of the human subclass IgG1 with the mutations I253A, H310A and H435A and the mutations T366W and optionally S354C in one of the antibody heavy chains and the mutations T366S, L368A, Y407V and optionally Y349C in the respective other antibody heavy chain,
e) both antibody heavy chains are full length antibody heavy chains of the human subclass IgG1 with the mutations M252Y, S254T and T256E and the mutations T366W and optionally S354C in one of the antibody heavy chains and the mutations T366S, L368A, Y407V and optionally Y349C in the respective other antibody heavy chain, or
f) both antibody heavy chains are antibody heavy chains of the human subclass IgG1 with the mutations T307H and N434H and the mutations T366W and optionally S354C in one of the antibody heavy chains and the mutations T366S, L368A, Y407V and optionally Y349C in the respective other antibody heavy chain, wherein the c-terminal lysine or glycine-lysine dipeptide can be present or absent independently in one or both heavy chains,
wherein the C-terminal lysine or glycine-lysine dipeptide can be present or absent independently of each other in one or both heavy chains.


SEQUENCE LISTING (66 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 441 aa, Homo sapiens
  MISC_FEATURE (422)..(422): X=phosphoserine
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDXPQLATLADEVSASLAKQGL

SEQ ID NO: 4 - protein, 42 aa, Homo sapiens [sequence omitted]

SEQ ID NO: 5 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 6 - protein, 7 aa, Oryctolagus cuniculus
GFSLSSY

SEQ ID NO: 7 - protein, 5 aa, Oryctolagus cuniculus
SYAMS

SEQ ID NO: 8 - protein, 5 aa, Oryctolagus cuniculus
WSGGS

SEQ ID NO: 9 - protein, 16 aa, Oryctolagus cuniculus
YIWSGGSTDYASWAKG

SEQ ID NO: 10 - protein, 16 aa, Artificial Sequence
  299-000 HVR-H2 Kabat G65S
YIWSGGSTDYASWAKS

SEQ ID NO: 11 - protein, 17 aa, Oryctolagus cuniculus
RYGTSYPDYGDANGFDP

SEQ ID NO: 12 - protein, 17 aa, Artificial Sequence
  299-000 HVR-H3 DASG
RYGTSYPDYGDASGFDP

SEQ ID NO: 13 - protein, 17 aa, Artificial Sequence
  299-000 HVR-H3 DAQG
RYGTSYPDYGDAQGFDP

SEQ ID NO: 14 - protein, 11 aa, Oryctolagus cuniculus
QASQSISSYLS

SEQ ID NO: 15 - protein, 11 aa, Artificial Sequence
  299-000 HVR-L1 RAA
RASQSISSYLA

SEQ ID NO: 16 - protein, 7 aa, Oryctolagus cuniculus
RASTLAS

SEQ ID NO: 17 - protein, 12 aa, Oryctolagus cuniculus
QQCYSSSNVDNT

SEQ ID NO: 18 - protein, 12 aa, Artificial Sequence
  299-000 HVR-L3 NYA
QQNYASSNVDNT

SEQ ID NO: 19 - protein, 122 aa, Artificial Sequence
  299-023 VH humanization variant_DASG
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSS

SEQ ID NO: 20 - protein, 110 aa, Artificial Sequence
  299-009 VL humanization variant_NYA
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIK

SEQ ID NO: 21 - protein, 119 aa, Artificial Sequence
  anti-CD20 antibody VH
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 22 - protein, 112 aa, Artificial Sequence
  anti-CD20 antibody VL
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 23 - protein, 126 aa, Homo sapiens [sequence omitted]

SEQ ID NO: 24 - protein, 108 aa, Homo sapiens [sequence omitted]

SEQ ID NO: 25 - protein, 116 aa, Artificial Sequence
  humanized anti-alpha synuclein acntibody 9E4 VH
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSS

SEQ ID NO: 26 - protein, 113 aa, Artificial Sequence
  humanized anti-alpha synuclein acntibody 9E4 VL
DIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEIK

SEQ ID NO: 27 - protein, 115 aa, Mus musculus
QVQLQQSGAELAKPGTSVQMSCKASGYTFTNYWMNWIKARPGQGLEWIGATNPNNGYTDYNQRFKDKAILTADKSSNTAYMHLSSLTSEDSAVYFCASGGHLAYWGQGTVVTVSA

SEQ ID NO: 28 - protein, 112 aa, Mus musculus
DVVMTQIPLYLSVSPGDQASISCRSSQSLFHSKGNTYLHWYLQKPGQSPKLLINRVSNRFSGVPDRFSGSGSGTDFTLKISGVEAEDLGVYFCSQSAHVPWTFGGGTKLEIR

SEQ ID NO: 29 - protein, 115 aa, Mus musculus
VQLQQSGPELVKPGTSVKISCKASGYSFTSYYIHWVKQSPGQGLEWIGWIYPGSGNTKYSEKFKGKATLTADTSSSTAYMQLSSLTSEDSAVYFCARDGCYGFAYWGQGTLVTVS

SEQ ID NO: 30 - protein, 111 aa, Mus musculus
DIVLTQSPASLAVSLGQRATISCKASQSVDYDGDSYMNWYQQKPGQPPKFLICAASNLESGIPARFSGSGSGTDFTLNIHPVEEEDAATYYCQQSNEDPFTFGSGTKLEIK

SEQ ID NO: 31 - protein, 115 aa, Oryctolagus cuniculus
QSVEESGGRLVTPGTPLTLTCTVSGFSINSYAMIWVRQAPGEGLEWIGVIYPSGNTYYANWAKGRFTVSRTSTTVDLKITSPTTEDTATYFCARRDGTDKTFNIWGPGTLVTVSL

SEQ ID NO: 32 - protein, 112 aa, Oryctolagus cuniculus
QVLTQTPSPVSAAVGGTVTINCQASQNVYGDNYLAWYQQKPGQPPKLLIYEASKLASGVPPRFSGSGSGTQFTLTISDVQCDDAATYYCQGEFLCTTSDCFTFGGGTGVVVR

SEQ ID NO: 33 - protein, 119 aa, Oryctolagus cuniculus
QSVEESGGRLVTPGTPLTLTCTVSGIDLSRYAMSWVRQAPGKGLEWIGVINSSGATYYASWAKGRFTISETSTTVELKITSPTTEDTATYFCARWTYDDYGDFQGFNIWGPGTLVTVSL

SEQ ID NO: 34 - protein, 111 aa, Oryctolagus cuniculus
AVLTQTPSPVSAAVGGTVTISCQSSQSVYNNNDLAWYQQKPGQPPKLLIYRASKLASGVPSRFKGSGSGTQFTLTISGVQCDDAATYYCLGGYDDDADMGAFGGGTEVVVK

SEQ ID NO: 35 - protein, 111 aa, Artificial Sequence
  heavy chain variable domain
QSLEESGGRLVTPGTPLTLTCTVSGIDLSRDTMIWVRQAPGEGLEWIGSIYTDSGNTWYASWVKGRFTISKTSSTTVDLRITSPTTEDTATYFCARNFSVWGPGTLVTVSL

SEQ ID NO: 36 - protein, 112 aa, Artificial Sequence
  light chain variable domain
QVLTQTPSPVSAAVGGTVTINCQASQSVYNSDRLAWFQQMRGQPPKLLIYDVSKLASGVPSRFSGSGSGTQFTLTISDVQCDDAATYYCLGGYDCSSAECNVFGGGTEVVVK

SEQ ID NO: 37 - protein, 20 aa, Artificial Sequence
  peptidic linker
GGGGSGGGGSGGGGSGGGGS

SEQ ID NO: 38 - protein, 32 aa, Artificial Sequence
  peptidic linker
GGGGSGGGGSGGGGSGGGGSGGGGSGGGGSGG

SEQ ID NO: 39 - protein, 448 aa, Artificial Sequence
  Anti-TfR-mAb HC
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, Artificial Sequence
  Anti-TfR-mAb LC
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 448 aa, Artificial Sequence
  anti-TfR one Fab mAb HC1
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 42 - protein, 241 aa, Artificial Sequence
  anti-TfR one Fab mAb HC 2
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGLPETGGSGSHHHHHH

SEQ ID NO: 43 - protein, 214 aa, Artificial Sequence; identical to SEQ ID NO: 40
  anti-TfR one Fab mAb LC

SEQ ID NO: 44 - protein, 940 aa, Artificial Sequence
  mBS-2Fab HC1
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 45 - protein, 456 aa, Artificial Sequence
  mBS-2Fab HC2
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - protein, 215 aa, Artificial Sequence
  mBS-2Fab LC
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 940 aa, Artificial Sequence
  dBS-2Fab HC
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 48 - protein, 234 aa, Artificial Sequence
  dBS-2Fab LC
MGWSCIILFLVATATGVHSDIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 448 aa, Artificial Sequence
  anti-TfR-mAb PGLALA HC1
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 448 aa, Artificial Sequence
  anti-TfR-mAb PGLALA HC2
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 51 - protein, 214 aa, Artificial Sequence; identical to SEQ ID NO: 40
  anti-TfR-mAb PGLALA LC

SEQ ID NO: 52 - protein, 933 aa, Artificial Sequence
  mBS-1Fab HC
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 53 - protein, 241 aa, Artificial Sequence
  mBS-1Fab HC2
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGLPETGGSGSHHHHHH

SEQ ID NO: 54 - protein, 220 aa, Artificial Sequence
  mBS-1Fab LC
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 711 aa, Artificial Sequence
  mBS-noFab HC
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 56 - protein, 241 aa, Artificial Sequence; identical to SEQ ID NO: 53
  mBS-noFab HC2

SEQ ID NO: 57 - protein, 330 aa, Homo sapiens [sequence omitted]

SEQ ID NO: 58 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 226 aa, Homo sapiens
  MISC_FEATURE (131)..(131): X=E or D
  MISC_FEATURE (133)..(133): X=M or L
  misc_feature (136)..(136): Xaa can be any naturally occurring amino acid
  misc_feature (138)..(138): Xaa can be any naturally occurring amino acid
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 226 aa, Artificial Sequence
  variant human Fc-region of the IgG1 isotype with a hole mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 62 - protein, 226 aa, Artificial Sequence
  variant human Fc-region of the IgG1 isotype with a knob mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 221 aa, Artificial Sequence
  variant human Fc-region of the IgG1 subclass with the mutations I253A, H310A and H435A
CPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPG

SEQ ID NO: 64 - protein, 221 aa, Artificial Sequence
  variant human Fc-region of the IgG1 subclass with the mutations H310A, H433A and Y436A
CPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALANHATQKSLSLSPG

SEQ ID NO: 65 - protein, 221 aa, Artificial Sequence
  variant human Fc-region of the IgG1 subclass with the mutations M252Y, S254T and T256E
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 228 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG